SEQUENCE LISTING (11 sequences)

SEQ ID NO: 1 - DNA, 6698 bp, synthetic construct
  source 1..6698: mol_type = other DNA; note = pCI-mCD19 retroviral vector DNA sequence
gtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcacgcgtgggcccgggatccaccggtcgactacaaggacgacgatgacaaggggcatcaccatcaccaccattagctagcgtttaaacttccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaccatgccctcccctctgcccgtgagcttcctgctgttcctgaccctggtgggaggcaggccccagaagagcctgctggtggaggtggaggaaggcggcaacgtggtgctgccctgtctgcctgatagctcccctgtgagcagcgagaagctggcctggtacaggggcaaccagagcacccctttcctggaactgagccctggaagccctggactgggcctgcacgtgggaagcctgggcatcctgctggtgatcgtgaacgtgtccgaccacatgggcggcttctatctgtgccagaagaggccccccttcaaagacatctggcagcccgcctggaccgtgaacgtggaggacagcggcgagatgttcaggtggaacgccagcgacgtcagggacctggactgcgacctgagaaacagaagctccggctcccacaggagcacaagcggcagccagctgtacgtgtgggctaaggaccaccctaaggtgtggggcacaaagcccgtgtgtgcccctaggggaagctccctcaaccagagcctgatcaaccaggacctgacagtggcccccggaagcaccctgtggctgagctgtggcgtgcctcctgtgcctgtggccaaaggatccatctcctggacccacgtgcaccccagaaggcccaacgtgagcctgctgagcctgagcctgggaggcgaacaccccgtgagggagatgtgggtgtggggcagcctgctgctgctgcctcaggctaccgccctggacgaaggcacctactactgtctgaggggcaacctgacaatcgagaggcacgtgaaggtgatcgctagaagcgccgtgtggctgtggctgctgaggaccggcggatggattgtgcccgtggtgaccctggtgtacgtgatcttctgcatggtgtccctggtggccttcctgtactgtcagagagccttcatcctcaggagaaagaggaagaggatgacatagtgattaacgcggccaattcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaa

SEQ ID NO: 2 - DNA, 8430 bp, synthetic construct
  source 1..8430: mol_type = other DNA; note = pCI-mCD19-PDP1 retroviral vector DNA sequence
gtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcacgcgtccgccaccatggagcggcggcggtgcgcctgtccgggctcctggagttgcccgccgggggctgtgatgccgcctcgcctgccgggaatcccagtcagaacgtccagtctgccactgttctctgatgccatgccagcaccaactcaactgttttttcctctcgtccgtaactgtgaactgagcagaatctatggcactgcatgttactgccaccacaaacatctctgctgttcaccaccatacattcctcagaatcgtctgaggtacacaccccatcctgcatatgctaccttttgtaggccacgggagaactggtggcagtatactcaaggaaggagatacgcttctacaccgcagaaattttacctcacacctccacaagtcaacagcatccttaaagctaatgaatacagcttcaaagtaccagaatttgatggcaaaaatgtcagttccattcttggatttgacagcaatcagctgcctgcaaatgcacccatagaggaccggagaagtgcagcaacctgcttgcagaccagagggatgctcttgggggtttttgatggtcatgcaggctgtgcttgttcccaggcagtcagtgaaagactcttctattatattgctgtttccttgttgccccatgagactttgctagagattgaaaatgcagtggagagtggtcgggcactgctacctatccttcagtggcacaagcaccccaatgattacttcagtaaggaggcgtccaaattgtatttcaacagcttgaggacttactggcaagaactcatagacctcaatactggagaatcagctgatattgatgttaaggaggctttaattaatgctttcaagagactcgataatgacatttcattggaggctcaagttggtgatcctaattcttttctaaattacctggtgcttcgggtagcattttctggggctactgcttgtgtggcccatgtagatggtgttgacctccatgtggctaacactggtgatagtagagccatgctaggtgtgcaagaagaagatggctcttggtcagcagtcacactctctaatgaccacaatgctcagaatgaaagagaactagagcgtctgaaactggaacacccaaaaaatgaggccaagagcgtggtaaagcaggaccggctgcttggcttgctgatgccctttagggcttttggagatgtaaagttcaaatggagcattgaccttcaaaagagagtgatagagtctggcccagaccagttgaatgacaatgaatacaccaagtttatccctcctaactatcatacacctccttatcttactgctgagccagaggtaacttatcacagattaaggccacaggataaattcctagtgttagcaactgatgggttgtgggagactatgcatagacaggatgtggttaggattgtgggtgaatacttaactggtatgcatcaccaacagccaatagcagttggtgggtacaaggtgactctgggacagatgcatggccttttaacagaaaggagagcaaagatgtcatcagtgtttgaggatcagaatgcagcaacccatctcattcgccatgctgtaggcaataatgaatttggggctgttgatcatgaacgactctctaaaatgcttagtcttcctgaagagcttgctcggatgtatagagatgacattacaatcattgtagttcagttcaattctcatgttgtaggggcataccaaaaccaggaacagtaaaccggtcgactacaaggacgacgatgacaaggggcatcaccatcaccaccattagctagcgtttaaacttccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaccatgccctcccctctgcccgtgagcttcctgctgttcctgaccctggtgggaggcaggccccagaagagcctgctggtggaggtggaggaaggcggcaacgtggtgctgccctgtctgcctgatagctcccctgtgagcagcgagaagctggcctggtacaggggcaaccagagcacccctttcctggaactgagccctggaagccctggactgggcctgcacgtgggaagcctgggcatcctgctggtgatcgtgaacgtgtccgaccacatgggcggcttctatctgtgccagaagaggccccccttcaaagacatctggcagcccgcctggaccgtgaacgtggaggacagcggcgagatgttcaggtggaacgccagcgacgtcagggacctggactgcgacctgagaaacagaagctccggctcccacaggagcacaagcggcagccagctgtacgtgtgggctaaggaccaccctaaggtgtggggcacaaagcccgtgtgtgcccctaggggaagctccctcaaccagagcctgatcaaccaggacctgacagtggcccccggaagcaccctgtggctgagctgtggcgtgcctcctgtgcctgtggccaaaggatccatctcctggacccacgtgcaccccagaaggcccaacgtgagcctgctgagcctgagcctgggaggcgaacaccccgtgagggagatgtgggtgtggggcagcctgctgctgctgcctcaggctaccgccctggacgaaggcacctactactgtctgaggggcaacctgacaatcgagaggcacgtgaaggtgatcgctagaagcgccgtgtggctgtggctgctgaggaccggcggatggattgtgcccgtggtgaccctggtgtacgtgatcttctgcatggtgtccctggtggccttcctgtactgtcagagagccttcatcctcaggagaaagaggaagaggatgacatagtgattaacgcggccaattcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaa

SEQ ID NO: 3 - DNA, 7992 bp, synthetic construct
  source 1..7992: mol_type = other DNA; note = pCI-mCD19-PDK1 retroviral vector DNA sequence
gtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcacgcgtccgccaccatgaggctggcaaggctgctgcggggcggcacgagcgtcaggccgctctgcgccgtcccctgcgccagccgtagcctggcctcggcctcgggttccgggccggcgtcggagcttggcgttccgggccaggtggacttctatgcgcgcttctcgccgtcgccactctccatgaagcagttcctggacttcgggtcagtgaatgcttgtgaaaagacctcgtttatgtttctgcgacaagagttgcctgttagattggcaaatataatgaaagaaatcagccttctccccgataatcttctcaggaccccatccgtacagctggtgcaaagttggtatatccaaagccttcaggagttgcttgattttaaagacaaaagtgctgaagatgctaaaactatttatgaattcacagacacagtgataaggatcagaaaccggcacaatgatgtcattcccaccatggcccagggtgtgactgaatacaaggagagcttcggggtggatcctgtcaccagccaaaatgttcagtactttttggatcgattctacatgagtcgcatctcaattagaatgctactcaaccagcactccttattgttcggtggaaaaggaagtccatctcatcgaaagcacattggaagcataaatccaaactgcgacgtggtggaggtcattaaagatggctatgagaacgctaggcggctttgtgatttgtattatgttaactctcctgaacttgaacttgaagaactaaatgcgaaatcaccaggacagacaatacaagtggtttatgtaccatcccatctctatcacatggtgtttgaactgttcaagaatgccatgagagcgaccatggagcaccacgcggacaaaggcgtttatcccccgattcaggttcacgtcacgctgggcgaggaggatctgactgtgaagatgagtgaccggggaggcggtgttccactgaggaagatcgacagactcttcaactacatgtactcaaccgcaccccggcctcgtgttgaaacgtcccgtgctgtgcccctggctgggtttggttacggattgcccatatcacgcctctatgcacagtacttccagggagacctaaagctgtattccttagagggctacgggacagatgcggttatctacattaaggctctgtcgacagaatccgtcgagagactccctgtgtacaataaagccgcctggaagcattacaaagccaaccacgaggctgacgactggtgtgtgcccagccgagagccgaaagatatgaccacattccgaagctcttaaaccggtcgactacaaggacgacgatgacaaggggcatcaccatcaccaccattagctagcgtttaaacttccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaccatgccctcccctctgcccgtgagcttcctgctgttcctgaccctggtgggaggcaggccccagaagagcctgctggtggaggtggaggaaggcggcaacgtggtgctgccctgtctgcctgatagctcccctgtgagcagcgagaagctggcctggtacaggggcaaccagagcacccctttcctggaactgagccctggaagccctggactgggcctgcacgtgggaagcctgggcatcctgctggtgatcgtgaacgtgtccgaccacatgggcggcttctatctgtgccagaagaggccccccttcaaagacatctggcagcccgcctggaccgtgaacgtggaggacagcggcgagatgttcaggtggaacgccagcgacgtcagggacctggactgcgacctgagaaacagaagctccggctcccacaggagcacaagcggcagccagctgtacgtgtgggctaaggaccaccctaaggtgtggggcacaaagcccgtgtgtgcccctaggggaagctccctcaaccagagcctgatcaaccaggacctgacagtggcccccggaagcaccctgtggctgagctgtggcgtgcctcctgtgcctgtggccaaaggatccatctcctggacccacgtgcaccccagaaggcccaacgtgagcctgctgagcctgagcctgggaggcgaacaccccgtgagggagatgtgggtgtggggcagcctgctgctgctgcctcaggctaccgccctggacgaaggcacctactactgtctgaggggcaacctgacaatcgagaggcacgtgaaggtgatcgctagaagcgccgtgtggctgtggctgctgaggaccggcggatggattgtgcccgtggtgaccctggtgtacgtgatcttctgcatggtgtccctggtggccttcctgtactgtcagagagccttcatcctcaggagaaagaggaagaggatgacatagtgattaacgcggccaattcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaa

SEQ ID NO: 4 - DNA, 1737 bp, synthetic construct
  source 1..1737: mol_type = other DNA; note = Mouse PDP1 protein coding sequence
atggagcggcggcggtgcgcctgtccgggctcctggagttgcccgccgggggctgtgatgccgcctcgcctgccgggaatcccagtcagaacgtccagtctgccactgttctctgatgccatgccagcaccaactcaactgttttttcctctcgtccgtaactgtgaactgagcagaatctatggcactgcatgttactgccaccacaaacatctctgctgttcaccaccatacattcctcagaatcgtctgaggtacacaccccatcctgcatatgctaccttttgtaggccacgggagaactggtggcagtatactcaaggaaggagatacgcttctacaccgcagaaattttacctcacacctccacaagtcaacagcatccttaaagctaatgaatacagcttcaaagtaccagaatttgatggcaaaaatgtcagttccattcttggatttgacagcaatcagctgcctgcaaatgcacccatagaggaccggagaagtgcagcaacctgcttgcagaccagagggatgctcttgggggtttttgatggtcatgcaggctgtgcttgttcccaggcagtcagtgaaagactcttctattatattgctgtttccttgttgccccatgagactttgctagagattgaaaatgcagtggagagtggtcgggcactgctacctatccttcagtggcacaagcaccccaatgattacttcagtaaggaggcgtccaaattgtatttcaacagcttgaggacttactggcaagaactcatagacctcaatactggagaatcagctgatattgatgttaaggaggctttaattaatgctttcaagagactcgataatgacatttcattggaggctcaagttggtgatcctaattcttttctaaattacctggtgcttcgggtagcattttctggggctactgcttgtgtggcccatgtagatggtgttgacctccatgtggctaacactggtgatagtagagccatgctaggtgtgcaagaagaagatggctcttggtcagcagtcacactctctaatgaccacaatgctcagaatgaaagagaactagagcgtctgaaactggaacacccaaaaaatgaggccaagagcgtggtaaagcaggaccggctgcttggcttgctgatgccctttagggcttttggagatgtaaagttcaaatggagcattgaccttcaaaagagagtgatagagtctggcccagaccagttgaatgacaatgaatacaccaagtttatccctcctaactatcatacacctccttatcttactgctgagccagaggtaacttatcacagattaaggccacaggataaattcctagtgttagcaactgatgggttgtgggagactatgcatagacaggatgtggttaggattgtgggtgaatacttaactggtatgcatcaccaacagccaatagcagttggtgggtacaaggtgactctgggacagatgcatggccttttaacagaaaggagagcaaagatgtcatcagtgtttgaggatcagaatgcagcaacccatctcattcgccatgctgtaggcaataatgaatttggggctgttgatcatgaacgactctctaaaatgcttagtcttcctgaagagcttgctcggatgtatagagatgacattacaatcattgtagttcagttcaattctcatgttgtaggggcataccaaaaccaggaacagtaa

SEQ ID NO: 5 - DNA, 1299 bp, synthetic construct
  source 1..1299: mol_type = other DNA; note = Mouse PDK1 protein coding sequence
atgaggctggcaaggctgctgcggggcggcacgagcgtcaggccgctctgcgccgtcccctgcgccagccgtagcctggcctcggcctcgggttccgggccggcgtcggagcttggcgttccgggccaggtggacttctatgcgcgcttctcgccgtcgccactctccatgaagcagttcctggacttcgggtcagtgaatgcttgtgaaaagacctcgtttatgtttctgcgacaagagttgcctgttagattggcaaatataatgaaagaaatcagccttctccccgataatcttctcaggaccccatccgtacagctggtgcaaagttggtatatccaaagccttcaggagttgcttgattttaaagacaaaagtgctgaagatgctaaaactatttatgaattcacagacacagtgataaggatcagaaaccggcacaatgatgtcattcccaccatggcccagggtgtgactgaatacaaggagagcttcggggtggatcctgtcaccagccaaaatgttcagtactttttggatcgattctacatgagtcgcatctcaattagaatgctactcaaccagcactccttattgttcggtggaaaaggaagtccatctcatcgaaagcacattggaagcataaatccaaactgcgacgtggtggaggtcattaaagatggctatgagaacgctaggcggctttgtgatttgtattatgttaactctcctgaacttgaacttgaagaactaaatgcgaaatcaccaggacagacaatacaagtggtttatgtaccatcccatctctatcacatggtgtttgaactgttcaagaatgccatgagagcgaccatggagcaccacgcggacaaaggcgtttatcccccgattcaggttcacgtcacgctgggcgaggaggatctgactgtgaagatgagtgaccggggaggcggtgttccactgaggaagatcgacagactcttcaactacatgtactcaaccgcaccccggcctcgtgttgaaacgtcccgtgctgtgcccctggctgggtttggttacggattgcccatatcacgcctctatgcacagtacttccagggagacctaaagctgtattccttagagggctacgggacagatgcggttatctacattaaggctctgtcgacagaatccgtcgagagactccctgtgtacaataaagccgcctggaagcattacaaagccaaccacgaggctgacgactggtgtgtgcccagccgagagccgaaagatatgaccacattccgaagctcttaa

SEQ ID NO: 6 - DNA, 1614 bp, synthetic construct
  source 1..1614: mol_type = other DNA; note = Human PDP1 protein coding sequence
atgccagcaccaactcaactgttttttcctctcatccgtaactgtgaactgagcaggatctatggcactgcatgttactgccaccacaaacatctctgttgttcctcatcgtacattcctcagagtcgactgagatacacacctcatccagcatatgctaccttttgcaggccaaaggagaactggtggcagtacacccaaggaaggagatatgcttccacaccacagaaattttacctcacacctccacaagtcaatagcatccttaaagctaatgaatacagtttcaaagtgccagaatttgacggcaaaaatgtcagttctatccttggatttgacagcaatcagctgcctgcaaatgcacccattgaggaccggagaagtgcagcaacctgcttgcagaccagagggatgcttttgggggtttttgatggccatgcaggttgtgcttgttcccaggcagtcagtgaaagactcttttattatattgctgtctctttgttaccccatgagactttgctagagattgaaaatgcagtggagagcggccgggcactgctacccattctccagtggcacaagcaccccaatgattactttagtaaggaggcatccaaattgtactttaacagcttgaggacttactggcaagagcttatagacctcaacactggtgagtcgactgatattgatgttaaggaggctctaattaatgccttcaagaggcttgataatgacatctccttggaggcgcaagttggtgatcctaattcttttctcaactacctggtgcttcgagtggcattttctggagccactgcttgtgtggcccatgtggatggtgttgaccttcatgtggccaatactggcgatagcagagccatgctgggtgtgcaggaagaggacggctcatggtcagcagtcacgctgtctaatgaccacaatgctcaaaatgaaagagaactagaacggctgaaattggaacatccaaagagtgaggccaagagtgtcgtgaaacaggatcggctgcttggcttgctgatgccatttagggcatttggagatgtaaagttcaaatggagcattgaccttcaaaagagagtgatagaatctggcccagaccagttgaatgacaatgaatataccaagtttattcctcctaattatcacacacctccttatctcactgctgagccagaggtaacttaccaccgattaaggccacaggataagtttctggtgttggctactgatgggttgtgggagactatgcataggcaggatgtggttaggattgtgggtgagtacctaactggcatgcatcaccaacagccaatagctgttggtggctacaaggtgactctgggacagatgcatggccttttaacagaaaggagaaccaaaatgtcctcggtatttgaggatcagaacgcagcaacccatctcattcgccacgctgtgggcaacaacgagtttgggactgttgatcatgagcgcctctctaaaatgcttagtcttcctgaagagcttgctcgaatgtacagagatgacattacaatcattgtagttcagttcaattctcatgttgtaggggcgtatcaaaaccaagaatag

SEQ ID NO: 7 - DNA, 1311 bp, synthetic construct
  source 1..1311: mol_type = other DNA; note = Human PDK1 protein coding sequence
atgaggctggcgcggctgcttcgcggagccgccttggccggcccgggcccggggctgcgcgccgccggcttcagccgcagcttcagctcggactcgggctccagcccggcgtccgagcgcggcgttccgggccaggtggacttctacgcgcgcttctcgccgtccccgctctccatgaagcagttcctggacttcggatcagtgaatgcttgtgaaaagacctcatttatgtttctgcggcaagagttgcctgtcagactggcaaatataatgaaagaaataagtctccttccagataatcttctcaggacaccatccgttcaattggtacaaagctggtatatccagagtcttcaggagcttcttgattttaaggacaaaagtgctgaggatgctaaagctatttatgactttacagatactgtgatacggatcagaaaccgacacaatgatgtcattcccacaatggcccagggtgtgattgaatacaaggagagctttggggtggatcctgtcaccagccagaatgttcagtactttttggatcgattctacatgagtcgcatttcaattagaatgttactcaatcagcactctttattgtttggtggaaaaggcaaaggaagtccatctcatcgaaaacacattggaagcataaatccaaactgcaatgtacttgaagttattaaagatggctatgaaaatgctaggcgtctgtgtgatttgtattatattaactctcccgaactagaacttgaagaactaaatgcaaaatcaccaggacagccaatacaagtggtttatgtaccatcccatctctatcacatggtgtttgaacttttcaagaatgcaatgagagccactatggaacaccatgccaacagaggtgtttacccccctattcaagttcatgtcacgctgggtaatgaggatttgactgtgaagatgagtgaccgaggaggtggcgttcctttgaggaaaattgacagacttttcaactacatgtattcaactgcaccaagacctcgtgttgagacctcccgcgcagtgcctctggctggttttggttatggattgcccatatcacgtctttacgcacaatacttccaaggagacctgaagctgtattccctagagggttacgggacagatgcagttatctacattaaggctctgtcaacagactcaatagaaagactcccagtgtataacaaagctgcctggaagcattacaacaccaaccacgaggctgatgactggtgcgtccccagcagagaacccaaagacatgacgacgttccgcagtgcctag

SEQ ID NO: 8 - protein, 578 aa, synthetic construct
  source 1..578: note = Mouse PDP1 amino acid sequence
MERRRCACPGSWSCPPGAVMPPRLPGIPVRTSSLPLFSDAMPAPTQLFFPLVRNCELSRIYGTACYCHHKHLCCSPPYIPQNRLRYTPHPAYATFCRPRENWWQYTQGRRYASTPQKFYLTPPQVNSILKANEYSFKVPEFDGKNVSSILGFDSNQLPANAPIEDRRSAATCLQTRGMLLGVFDGHAGCACSQAVSERLFYYIAVSLLPHETLLEIENAVESGRALLPILQWHKHPNDYFSKEASKLYFNSLRTYWQELIDLNTGESADIDVKEALINAFKRLDNDISLEAQVGDPNSFLNYLVLRVAFSGATACVAHVDGVDLHVANTGDSRAMLGVQEEDGSWSAVTLSNDHNAQNERELERLKLEHPKNEAKSVVKQDRLLGLLMPFRAFGDVKFKWSIDLQKRVIESGPDQLNDNEYTKFIPPNYHTPPYLTAEPEVTYHRLRPQDKFLVLATDGLWETMHRQDVVRIVGEYLTGMHHQQPIAVGGYKVTLGQMHGLLTERRAKMSSVFEDQNAATHLIRHAVGNNEFGAVDHERLSKMLSLPEELARMYRDDITIIVVQFNSHVVGAYQNQEQ

SEQ ID NO: 9 - protein, 432 aa, synthetic construct
  source 1..432: note = Mouse PDK1 amino acid sequence
MRLARLLRGGTSVRPLCAVPCASRSLASASGSGPASELGVPGQVDFYARFSPSPLSMKQFLDFGSVNACEKTSFMFLRQELPVRLANIMKEISLLPDNLLRTPSVQLVQSWYIQSLQELLDFKDKSAEDAKTIYEFTDTVIRIRNRHNDVIPTMAQGVTEYKESFGVDPVTSQNVQYFLDRFYMSRISIRMLLNQHSLLFGGKGSPSHRKHIGSINPNCDVVEVIKDGYENARRLCDLYYVNSPELELEELNAKSPGQTIQVVYVPSHLYHMVFELFKNAMRATMEHHADKGVYPPIQVHVTLGEEDLTVKMSDRGGGVPLRKIDRLFNYMYSTAPRPRVETSRAVPLAGFGYGLPISRLYAQYFQGDLKLYSLEGYGTDAVIYIKALSTESVERLPVYNKAAWKHYKANHEADDWCVPSREPKDMTTFRSS

SEQ ID NO: 10 - protein, 537 aa, synthetic construct
  source 1..537: note = Human PDP1 amino acid sequence
MPAPTQLFFPLIRNCELSRIYGTACYCHHKHLCCSSSYIPQSRLRYTPHPAYATFCRPKENWWQYTQGRRYASTPQKFYLTPPQVNSILKANEYSFKVPEFDGKNVSSILGFDSNQLPANAPIEDRRSAATCLQTRGMLLGVFDGHAGCACSQAVSERLFYYIAVSLLPHETLLEIENAVESGRALLPILQWHKHPNDYFSKEASKLYFNSLRTYWQELIDLNTGESTDIDVKEALINAFKRLDNDISLEAQVGDPNSFLNYLVLRVAFSGATACVAHVDGVDLHVANTGDSRAMLGVQEEDGSWSAVTLSNDHNAQNERELERLKLEHPKSEAKSVVKQDRLLGLLMPFRAFGDVKFKWSIDLQKRVIESGPDQLNDNEYTKFIPPNYHTPPYLTAEPEVTYHRLRPQDKFLVLATDGLWETMHRQDVVRIVGEYLTGMHHQQPIAVGGYKVTLGQMHGLLTERRTKMSSVFEDQNAATHLIRHAVGNNEFGTVDHERLSKMLSLPEELARMYRDDITIIVVQFNSHVVGAYQNQE

SEQ ID NO: 11 - protein, 436 aa, synthetic construct
  source 1..436: note = Human PDK1 amino acid sequence
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYDFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

(19) United States
(12) Patent Application Publication
(10) Pub. No.: US 2026/0174850 A1
(43) Pub. Date: Jun. 25, 2026

(54) ENGINEERED T CELLS HAVING ALTERED PYRUVATE METABOLISM, METHODS FOR THEIR PRODUCTION AND USE IN THERAPY, E.G., FOR TREATMENT OF CANCER OR INFECTION

(71) Applicant: THE TRUSTEES OF DARTMOUTH COLLEGE, HANOVER, NH (US)

(72) Inventors: Edward Usherwood, Hanover, NH (US); Taewook Kang, Lebanon, NH (US); Young-Kwang Usherwood, Hanover, NH (US)

(21) Appl. No.: 19/126,670

(22) PCT Filed: Nov. 2, 2023
(86) PCT No.: PCT/US2023/078435

§ 371 Date: May 2, 2025

Related U.S. Application Data
(60) Provisional application No. 63/381,994, filed on Nov. 2, 2022.

(51) Int. Cl. A61K 40/11; C12N 5/0783

(52) U.S. Cl. CPC A61K 40/11; C12N 5/0638

(57) ABSTRACT

The invention provides methods for upregulating mitochondrial and/or glycolytic metabolism in T cells by increasing the flux of pyruvate into the citric acid cycle, thereby increasing mitochondrial and/or glycolytic metabolism. In general these methods comprise stable expression of a transgene encoding either pyruvate dehydrogenase kinase enzyme and/or pyruvate dehydrogenase phosphatase enzyme in T cells. The invention further provides engineered T cells, preferably engineered human T cells produced by such methods, which optionally are further engineered, e.g., to express a targeting and/or therapeutic moiety and the use thereof and compositions containing in adoptive T cell therapies, e.g., the treatment and prevention of cancer, infection, inflammation, autoimmunity and neurodegenerative conditions.

### RELATED APPLICATIONS

The present application claims benefit of priority to U.S. Provisional Application No. 63/381,994, filed on Nov. 2, 2022, the contents of which are incorporated by reference in their entirety.

### GOVERNMENT INTEREST

FEDERALLY SPONSORED RESEARCH (1) This invention was made with government support under grant numbers R01 AI155015 and R01 A1122854, both awarded by National Institutes of Health. The government has certain rights in the invention.

### FIELD OF THE INVENTION

The present disclosure generally relates to T cells, e.g., primary CD8+ and/or CD4+ T cells which are genetically engineered to upregulate mitochondrial and/or glycolytic metabolism; methods for their production; and methods for their use in cell therapy, e.g., treatment of cancer, particularly solid tumors, infectious conditions, and chronic inflammatory or autoimmune conditions. These engineered T cells possess advantages which render them well suited for usage in adoptive T cell therapies including e.g., increased survival, increased effector functions, and enhanced secondary response of memory CD8+ T cells upon antigen re-exposure.

### BACKGROUND

The technique of Adoptive T cell Therapy (ACT) is revolutionizing treatment of cancer (Met, et al., Seminars in Immunopathology, 41(1):49-58). In such methods, T cells, e.g., CD8+ T cells are harvested from a patient or donor, expanded and stimulated ex vivo, then infused into the patient. One source of CD8+ T cells is a tumor from the patient. Tumor-infiltrating lymphocytes (TIL) are isolated from the patient's tumors and naturally have specificity for the patient's tumor cells. Bone marrow from the patient is another source of lymphocytes with pre-existing specificity for the tumor. In other cases, CD8+ T cells are harvested from peripheral blood.

A particularly powerful form of ACT comprises the engineering of T cells, e.g., CD8+ T cells or CD4+ T cells so that they express a receptor for an antigen on the surface of tumor cells. One such receptor is a Chimeric Antigen Receptor (CAR). A CAR comprises the antigen-binding portion of an antibody or the ligand binding portion of a receptor and the signaling components of one or more immunoreceptors and/or costimulatory molecules. Another specific antigen receptor which may be engineered into T cells is a T cell Receptor (TCR), e.g., one comprising tumor antigen-specific a and 3 chains.

To create such engineered tumor-specific cells, T cells, e.g., CD8+ T cells or CD4+ T cells, donor T cells, e.g., CD8+ T cells or CD4+ T cells are harvested, most often from peripheral blood, and genetically modified to express an exogenous receptor, e.g., a CAR and/or it is another TCR e.g., a naturally-occurring human T cell receptor from another subject.

ACT has demonstrated impressive effectiveness in some blood cancers, but it is not effective for all cancer types, particularly it is typically not efficacious in treating solid tumors (Morgan & Schambach, (2018), Frontiers in immunology, 9:2493). Another limitation of current T cell therapies is that effector CD8+ T cells are used which metabolize glucose via anaerobic glycolysis, and in the glucose poor environment of a solid tumor such T cells often do not function efficiently.

The pathways of anaerobic glycolysis and aerobic oxidative phosphorylation diverge at the fate of pyruvate. If the cell is utilizing anaerobic glycolysis, pyruvate is reduced to lactate and excreted from the cell. If the cell is utilizing aerobic oxidative phosphorylation, pyruvate dehydrogenase (PDH) oxidizes and decarboxylates pyruvate to form acetyl-CoA which is fed into the tricarboxylic acid cycle (TCA, see FIG. 1). The activity of PDH is controlled by pyruvate dehydrogenase kinase (PDK), which phosphorylates and inactivates PDH, and pyruvate dehydrogenase phosphatase (PDP), which dephosphorylates the phosphorylated PDH and restores its activity.

T cells which have been engineered in order to induce a stable, heritable genetic change in their pyruvate metabolism cells or methods for their production are not currently available. This is important, as only genetic changes are passed on to daughter cells as T cells proliferate and expand ex vivo or in vivo.

Accordingly, T cells having improved mitochondrial and/or glycolytic metabolism and methods for their production are desperately needed.

### BRIEF SUMMARY AND INVENTIVE OBJECTS

As noted above, T cells having improved mitochondrial and/or glycolytic metabolism and methods for their production are desperately needed. The present invention addresses this substantial need in the art by solving the problem of T cells relying on inefficient anaerobic glycolysis in the tumor microenvironment and thereby losing their competition for glucose (which is metabolized by rapidly proliferating tumor cells) by providing T cells having enhanced mitochondrial and/or glycolytic activity. As exhausted T cells have less functional mitochondria, augmenting mitochondrial and/or glycolytic metabolism should alleviate mitochondrial and/or glycolytic dysfunction, and thereby reduceTcell exhaustion at diseased sites e.g., in solid tumors or sites of inflammation such as in joints, sites of autoimmunity, or infection sites thereby enhancing the efficacy of adoptive T cell-mediated therapies.

In particular, the present invention provides genetically engineered T cells, preferably primary human T cells, and more preferably primary human CD8+ and/or CD4+ T cells which are genetically modified to provide stable, heritable expression of pyruvate dehydrogenase kinase enzyme (PDK) and/or pyruvate dehydrogenase phosphatase (PDP) enzyme in said cells.

Further the present invention provides methods for generating such genetically engineered T cells, preferably primary human T cells, and more preferably primary human CD8+ and/or CD4+ T cells which are genetically modified to provide stable, heritable expression of pyruvate dehydrogenase kinase enzyme (PDK) and/or pyruvate dehydrogenase phosphatase (PDP) enzyme.

Also, the present invention pharmaceutical compositions comprising genetically engineered T cells, preferably primary human T cells, and more preferably primary human CD8+ and/or CD4+ T cells which are genetically modified to provide stable, heritable expression of pyruvate dehydrogenase kinase enzyme (PDK) and/or pyruvate dehydrogenase phosphatase (PDP) enzyme.

Additionally, the invention provides methods of therapy comprising the administration of genetically engineered T cells, preferably primary human T cells, and more preferably primary human CD8+ and/or CD4+ T cells, and pharmaceutical compositions comprising same wherein said Tcells are genetically modified to provide stable, heritable expression of pyruvate dehydrogenase kinase enzyme (PDK) and/or pyruvate dehydrogenase phosphatase (PDP) enzyme.

In particular the present invention provides three methods for genetically modifying T cells in order to upregulate mitochondrial metabolism therein by increasing the flux of pyruvate into the citric acid cycle and thereby increasing mitochondrial metabolism. These three methods all involve introducing at least one genetic construct into T cells, preferably primary human T cells, and more preferably primary human CD8+ and/or CD4+ T cells, as follows:
(i) introducing at least one genetic construct, e.g., a retroviral construct, which provides for the stable, heritable expression of pyruvate dehydrogenase kinase enzyme (PDK);
(ii) introducing at least one genetic construct, e.g., a retroviral construct, which provides for the stable, heritable expression of pyruvate dehydrogenase phosphatase (PDP) enzyme; or
(iii) introducing at least one genetic construct, e.g., a retroviral construct, which provides for the stable, heritable expression of pyruvate dehydrogenase kinase enzyme (PDK) and pyruvate dehydrogenase phosphatase (PDP) enzyme.

The above methods may further optionally comprise introducing at least one other genetic modification into the T cell which provides for the expression, absence of expression and/or impaired expression of one or more other gene products such as a CAR, therapeutic polypeptide, immunomodulatory polypeptide or a marker. In exemplary embodiments genetically engineered T cells according to the invention are obtained using retroviral constructs which upon introduction into T cells, preferably primary human T cells, and more preferably primary human CD8+ and/or CD4+ T cells, provide for stable and heritable retrovirally-mediated expression of pyruvate dehydrogenase kinase enzyme and/or retrovirally mediated expression of pyruvate dehydrogenase phosphatase enzyme and/or pyruvate dehydrogenase phosphatase (PDP) enzyme in such T cells. As described in detail infra, these T are useful in adoptive T cell therapies, e.g., for treating cancer, infectious conditions, inflammatory conditions and/or autoimmune conditions in subjects in need thereof as they should possess enhanced and more prolonged potency compared to T cells currently used in adoptive T cell therapies. In particular, genetically engineered T cells according to the invention should be well suited for treatment of conditions which are often recalcitrant to adoptive T cell therapies, e.g., treatment of solid tumors and/or cancers which have relapsed and/or metastasized.

It is an object of the invention to provide isolated or recombinant T cells, optionally human primary T cells, further optionally human primary CD4+ or CD8+ T cells, which are engineered to stably and/or heritably express increased amounts of pyruvate dehydrogenase kinase enzyme (PDK) and/or pyruvate dehydrogenase phosphatase (PDP) enzyme, or progeny thereof.

It is an object of the invention to provide isolated or recombinant T cells, optionally human primary T cells, further optionally human primary CD4+ or CD8+ T cells, which are engineered to stably and/or heritably express increased amounts of pyruvate dehydrogenase kinase enzyme (PDK) and/or pyruvate dehydrogenase phosphatase (PDP) enzyme, or progeny thereof wherein the expression of a gene encoding said pyruvate dehydrogenase kinase enzyme (PDK) and/or pyruvate dehydrogenase phosphatase (PDP) enzyme is virally mediated.

It is an object of the invention to provide isolated or recombinant T cells, optionally human primary T cells, further optionally human primary CD4+ or CD8+ T cells, which are engineered to stably and/or heritably express increased amounts of pyruvate dehydrogenase kinase enzyme (PDK) and/or pyruvate dehydrogenase phosphatase (PDP) enzyme, or progeny thereof wherein the expression of a gene encoding said pyruvate dehydrogenase kinase enzyme (PDK) and/or pyruvate dehydrogenase phosphatase (PDP) enzyme is mediated by a retroviral, lentiviral, adenoviral, adeno-associated viral or herpes simplex viral vector sequence.

It is an object of the invention to provide isolated or recombinant T cells, optionally human primary T cells, further optionally human primary CD4+ or CD8+ T cells, wherein the expression of a gene encoding said pyruvate dehydrogenase kinase enzyme (PDK) and/or pyruvate dehydrogenase phosphatase (PDP) enzyme is retrovirally mediated.

It is an object of the invention to provide isolated or recombinant T cells, optionally human primary T cells, further optionally human primary CD4+ or CD8+ T cells, wherein the gene encoding said pyruvate dehydrogenase kinase enzyme (PDK) and/or pyruvate dehydrogenase is introduced by use of a gene editing technique.

It is an object of the invention to provide isolated or recombinant T cells, further optionally human primary CD4+ or CD8+ T cells, wherein said gene editing method comprises the use of TALEN (transcription activator-like effector nuclease) mediated gene editing, CRSPR (Clustered Regularly Interspaced Short Palindromic Repeats) mediated gene editing, double strand repair, meganuclease mediated gene editing, zinc finger nuclease mediated gene editing, Nucleobase Modification (BASE Editing) or ARCUT (artificial restriction DNA cutter) mediated gene editing.

It is an object of the invention to provide isolated or recombinant T cells, according to any one of the foregoing, which are expanded and/or permitted to proliferate in vitro.

It is an object of the invention to provide isolated or recombinant T cells, further optionally human primary CD4+ or CD8+ T cells, according to any one of the foregoing, which comprise multiple copies of a gene encoding pyruvate dehydrogenase kinase enzyme (PDK) and/or pyruvate dehydrogenase phosphatase (PDP) enzyme.

It is an object of the invention to provide isolated or recombinant T cells, optionally human primary CD4+ or CD8+ T cells, according to any one of the foregoing, wherein the expression of a gene encoding said pyruvate dehydrogenase kinase enzyme (PDK) and/or pyruvate dehydrogenase phosphatase (PDP) enzyme is controlled by a constitutive promoter.

It is an object of the invention to provide isolated or recombinant T cells, optionally human primary CD4+ or CD8+ T cells, according to any one of the foregoing, wherein the expression of a gene encoding said pyruvate dehydrogenase kinase enzyme (PDK) and/or pyruvate dehydrogenase phosphatase (PDP) enzyme is controlled by an inducible promoter.

It is an object of the invention to provide isolated or recombinant T cells, optionally human primary CD4+ or CD8+ T cells, according to any one of the foregoing, wherein the expression of a gene encoding said pyruvate dehydrogenase kinase enzyme (PDK) and/or pyruvate dehydrogenase phosphatase (PDP) enzyme is controlled by a tissue-specific promoter.

It is an object of the invention to provide isolated or recombinant T cells, according to any one of the foregoing, further optionally human primary CD4+ or CD8+ T cells, according to any one of the foregoing, wherein the expression of a gene encoding said pyruvate dehydrogenase kinase enzyme (PDK) and/or pyruvate dehydrogenase phosphatase (PDP) enzyme is controlled by a promoter selected from cytomegalovirus enhancer/chicken β-actin (CAG), CMV promoter, PGK promoter, a MSCV promoter. and elongation factor (EF)-1α promoters, MSCV promoter, EFS promoter.

It is an object of the invention to provide isolated or recombinant T cells, according to any one of the foregoing, further optionally human primary CD4+ or CD8+ T cells, according to any one of the foregoing, which further comprise an inducible suicide gene.

It is an object of the invention to provide isolated or recombinant T cells, according to any one of the foregoing, further optionally human primary CD4+ or CD8+ T cells, according to any one of the foregoing, which are derived from one or more of: peripheral T cells, bone marrow T cells, Tumor Infiltrating Lymphocytes, autologous T cells, allogeneic T cells, hematopoietic stem cells, and/or induced pluripotent stem cells.

It is an object of the invention to provide isolated or recombinant T cells, according to any one of the foregoing, wherein the T cells comprise CD4+ T cells and/or CD8+ T cells, optionally primary human CD4+ T cells and/or CD8+ T cells or progeny thereof.

It is an object of the invention to provide isolated or recombinant T cells, according to any one of the foregoing, wherein the T cells comprise CD4+ T cells and/or CD8+ T cells, wherein the expressed Pdp1 comprises an amino acid sequence possessing at least 80, 85, 90, 91, 92, 93, 94, 95, 96, 97, 98, 99 or 100% sequence identity to the human Pdp1 of SEQ ID NO: 10 or the murine Pdp1 of SEQ ID NO: 8.

It is an object of the invention to provide isolated or recombinant T cells, according to any one of the foregoing, wherein the T cells comprise CD4+ T cells and/or CD8+ T cells, wherein the expressed Pdp1 polypeptide comprises an amino acid sequence possessing at least 80, 85, 90, 91, 92, 93, 94, 95, 96, 97, 98, 99 or 100% sequence identity to the human Pdk1 of SEQ ID NO: 11 or the murine Pdk1 of SEQ ID NO: 9.

It is an object of the invention to provide isolated or recombinant T cells, according to any one of the foregoing, wherein the T cells comprise CD4+ T cells and/or CD8+ T cells, wherein the nucleic acid sequence encoding Pdp1 comprises a nucleic acid sequence possessing at least 80, 85, 90, 91, 92, 93, 94, 95, 96, 97, 98, 99 or 100% sequence identity to the human Pdk1 nucleic acid sequence of SEQ ID NO: 7 or the murine Pdk1 nucleic acid sequence of SEQ ID NO: 5.

It is an object of the invention to provide isolated or recombinant T cells, according to any one of the foregoing, wherein the T cells comprise CD4+ T cells and/or CD8+ T cells, wherein the nucleic acid encoding Pdp1 comprises a nucleic acid sequence possessing at least 80, 85, 90, 91, 92, 93, 94, 95, 96, 97, 98, 99 or 100% sequence identity to the human Pdp1 nucleic acid sequence of SEQ ID NO: 6 or the murine Pdp1 nucleic acid sequence of SEQ ID NO: 4.

It is an object of the invention to provide isolated or recombinant T cells, according to any one of the foregoing, wherein the T cells comprise CD4+ T cells and/or CD8+ T cells, wherein:
(i) said T cells are more metabolically active, or
(ii) said T cells have an increased rate of glycolysis, or
(iii) said T cells have an increased rate of oxidative phosphorylation, or
(iv) said T cells exhibit more active effector functions comprising production of Granzyme B, TNFα, and/or Interferon γ.

It is an object of the invention to provide isolated or recombinant T cells, according to any one of the foregoing, wherein the T cells are further engineered to comprise a desired targeting or therapeutic moiety, optionally wherein the desired targeting or therapeutic moiety comprises:
(i) a chimeric antigen receptor, or
(ii) an exogenous T cell receptor, or
(iii) a cytokine, or
(iv) a cytokine receptor, or
(v) a chemokine, or
(vi) a chemokine receptor,
(vii) an immune checkpoint modulator,
(viii) or a combination of any of the foregoing.

It is an object of the invention to provide isolated or recombinant T cells, according to any one of the foregoing, wherein the T cells are further engineered to comprise a desired targeting or therapeutic moiety, optionally wherein the desired targeting or therapeutic moiety comprises a chimeric antigen receptor(s) or exogenous TCR(s) that targets an antigen expressed by a tumor, site of infection, site of inflammation, and/or a site of autoimmunity in a treated subject, optionally wherein the chimeric antigen receptor(s) or exogenous TCR(s) targets one or more of Alphafetoprotein, BCMA, B7-H3, Carcinoembryonic antigen, CA-135, CD19, CD133, Claudin 18.2, c-Met, EGFR, FAP, GD2, GPC3, HER-2, MAGE, Mesothelin, MUC-1, MUC-16, NY-ESO-1, PD-L1, PSCA, PSMA, ROR1, or Tyrosinase, or wherein the immune checkpoint modulator comprises an antagonist antibody to one or more of: PD-1, PD-L1, PD-L2, CTLA-4, TIM-3, LAG-3, CD47, SIRPα, B7-H3.

It is an object of the invention to provide a composition comprising isolated or recombinant T cells or progeny of any one of the foregoing.

It is an object of the invention to provide a lyophilized composition comprising isolated or recombinant T cells or progeny of any one of the foregoing.

It is an object of the invention to provide a composition according to any of the foregoing which further comprises a therapeutic agent, optionally a cytokine, cytokine receptor, chemokine, chemokine receptor, immune checkpoint inhibitor agonist or antagonist,

It is an object of the invention to provide a composition according to any of the foregoing which further comprises a hormone, cytokine, cytokine antagonist, therapeutic antibody, immunostimulatory antibody or fusion protein, immunoinhibitory antibody or fusion protein, anti-inflammatory agent, checkpoint inhibitor or checkpoint inhibitor agonist or antagonist, e.g., a polypeptide which promotes or reduces the expression or activity of a stimulatory immune checkpoint molecule such as CD27, CD28, CD40, CD122, CD137, OX40, GITR or ICOS and/or a polypeptide which promotes or reduces the expression or activity of an inhibitory immune checkpoint molecule such as A2AR, A2BR, B7-H3, B7-H4, BTLA, CTLA-4, IDO, KIR, LAG3, NOX2, PD-1, PD-L1, VISTA or SIGLEC7).

It is an object of the invention to provide a composition according to any of the foregoing which further comprises and/or the engineered T cells of the invention are further engineered to comprise and express a gene encoding one of IL-2, IL-4, IL-5, IL-7, IL-10, IL-12p40, IL-12p70, IL-15, and interferon (IFN) gamma.

It is an object of the invention to provide a composition according to any of the foregoing which further comprises and/or the engineered T cells of the invention are further engineered to comprise and express a gene encoding one of a pro-inflammatory cytokine, e.g., IL-1a, IL-1b, IL-6, IL-13, IL-17a, tumor necrosis factor (TNF)-alpha TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF).

It is an object of the invention to provide a method for producing isolated or recombinant T cells of any one of the foregoing, comprising:
(i) inserting a DNA sequence which encodes either a PDP1 gene or a PDK1 into a vector,
(ii) contacting the vector with a T cell to form or produce a transduced T cell, (iii) growing the transduced T cells,
(iv) harvesting and/or purifying the transduced cells,
(v) optionally expanding or permitting the harvested cells to proliferate in vitro; and
(vi) further optionally freezing said cells so as to preserve their viability.

It is an object of the invention to provide a method for producing isolated or recombinant T cells of any one of the foregoing, comprising:
(i) inserting a DNA sequence which encodes either a PDP1 gene or a PDK1 into a DNA comprising a viral vector genome,
(ii) transfecting that genome into cultured mammalian cells,
(iii) culturing said cells to produce virus particles comprising said PDP1 or PDK1 gene,
(iv) harvesting and/or purifying said virus particles from the culture medium,
(v) contacting said virus particles with a T cell to form or produce a virally-transduced T cell,
(vi) growing the viral-transduced T cells,
(vii) harvesting and/or purifying the virally-transduced cells,
(viii) optionally expanding or permitting the cells to proliferate in vitro; and
(ix) and further optionally, freezing said cells so as to preserve their viability.

It is an object of the invention to provide a method as above, wherein the vector or viral vector genome is selected from a retroviral, lentiviral, adenoviral, adeno-associated viral or herpes simplex viral vector or genome sequence.

It is an object of the invention to provide a method as above, wherein the vector or viral vector genome is a retroviral vector or retroviral genome.

It is an object of the invention to provide a composition comprising isolated or recombinant T cells produced by any one of the foregoing methods.

It is an object of the invention to provide a composition comprising isolated or recombinant Tcells produced by any one of the foregoing methods, which comprises:
(i) DMSO in a concentration of 5-10% by volume, and/or
(ii) serum albumin from the same species as the recipient organism in a concentration of 0.1-5% by weight.

It is an object of the invention to provide a method of treatment or prevention of a condition or disease or at least one symptom associated therewith in a subject in need thereof comprising administering a therapeutically or prophylactically effective amount isolated or recombinant T cells according to any one of the foregoing or the compositions of any one of the foregoing, optionally wherein the dose includes no more than about 1×10⁸ total engineered cells, e.g., in the range of about 1×10⁶ to 1×10⁸ such cells, such as 2×10⁶, 5×10⁶, 1×10⁷, 5×10⁷, or 1×10⁸ cells.

It is an object of the invention to provide a method of treatment or prevention of a condition or disease or at least one symptom associated therewith in a subject in need thereof as above, in which the cells are administered by intravenous, intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, intrapleural, and/or intratumoral injection or infusion.

It is an object of the invention to provide a method of treatment or prevention of a condition or disease or at least one symptom associated therewith in a subject in need thereof as above, wherein the condition or disease is at least one cancer.

It is an object of the invention to provide a method of treatment or prevention of a condition or disease or at least one symptom associated therewith in a subject in need thereof as above, wherein the condition or disease comprises a solid tumor, cancer reoccurrence and/or cancer metastasis.

It is an object of the invention to provide a method of treatment or prevention of a condition or disease or at least one symptom associated therewith in a subject in need thereof as above, wherein the at least one cancer is selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

It is an object of the invention to provide a method of treatment or prevention of a condition or disease or at least one symptom associated therewith in a subject in need thereof as above, wherein the treatment prevents or inhibits cancer reoccurrence.

It is an object of the invention to provide a method of treatment or prevention of a condition or disease or at least one symptom associated therewith in a subject in need thereof as above, wherein the condition or disease is a neurodegenerative condition.

It is an object of the invention to provide a method of treatment or prevention of a condition or disease or at least one symptom associated therewith in a subject in need thereof as above, wherein the neurodegenerative condition comprises: Adrenoleukodystrophy, Alpers' Disease, Alpha-methylacyl-CoA racemase deficiency, Alzheimer's disease and other memory disorders, Amyotrophic lateral sclerosis, Andermann syndrome, Ataxia neuropathy spectrum, Ataxia-telangiectasia, Autosomal dominant cerebellar ataxia, deafness, and narcolepsy, Balo concentric sclerosis, Batten Disease, Central pontine myelinolysis, Cerebro-Oculo-Facio-Skeletal Syndrome, Congenital insensitivity to pain with anhidrosis, Corticobasal degeneration, Creutzfeldt-Jakob disease, Familial encephalopathy with neuroserpin inclusion bodies, Fatty acid hydroxylase-associated neurodegeneration, Friedreich ataxia, Frontotemporal dementia, Gerstmann-Straussler-Scheinker disease, GM2-gangliosidosis, Hemiballismus, Hereditary sensory and autonomic neuropathy, Huntington's disease, Juvenile primary lateral sclerosis, Leigh's Disease, Lewy body disease, Marinesco-Sjögren syndrome, Meige's syndrome, Mitochondrial membrane protein-associated neurodegeneration, Monomelic Amyotrophy, Motor neuron disease, Multiple sclerosis, Multiple system atrophy, Neurodegeneration with brain iron accumulation, Neuromyelitis optica, Opsoclonus Myoclonus, Pantothenate kinase-associated neurodegeneration, Parkinson's disease, Pelizaeus-Merzbacher disease, Polycystic lipomembranous osteodysplasia with sclerosing leukoencephalopathy, Posterior cortical atrophy, Primary lateral sclerosis, Primary progressive aphasia, Progressive bulbar palsy, Progressive external ophthalmoplegia, Progressive Multifocal Leukoencephalopathy, Progressive muscular atrophy, Progressive supranuclear palsy, Pseudobulbar palsy, Riboflavin transporter deficiency neuronopathy, Sandhoff disease, Spastic paraplegia, Spinal muscular atrophy, Reye syndrome, Striatonigral degeneration, and Spasmodic torticollis

It is an object of the invention to provide a method of treatment or prevention of a condition or disease or at least one symptom associated therewith in a subject in need thereof as above, wherein the condition or disease is an inflammatory condition.

It is an object of the invention to provide a method of treatment or prevention of a condition or disease or at least one symptom associated therewith in a subject in need thereof as above, wherein the at least one inflammatory condition or disease is selected from single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, fibrosis, ophthalmic inflammation, dermatologic inflammation, gastrointestinal inflammation, tendinopathies, pancreatitis, hepatitis, inflammatory arthritis, multiple sclerosis, Acute Respiratory Distress Syndrome (ARDS), wound healing, diabetic ulcers, non-healing wounds, lupus, Acne vulgaris, Allergy, Asthma, Ankylosing spondylitis, Antiphospholipid antibody syndrome, Atherosclerosis, Atopic dermatitis, Autoimmune diseases, Autoimmune encephalitis, Autoinflammatory diseases, Celiac disease, Chediak-Higashi syndrome, Chronic granulomatous disease, Chronic prostatitis, Chronic recurrent multifocal osteomyelitis, Colitis, Dermatomyositis, Diverticulitis, Endometriosis, Familial Mediterranean Fever, Fatty liver disease, Glomerulonephritis, Gout, Henoch-Schonlein purpura, Hidradenitis suppurativa, Hypersensitivities, Inclusion body myositis, Inflammatory bowel diseases, Interstitial cystitis, Juvenile dermatomyositis, Juvenile idiopathic arthritis, Juvenile lupus, Juvenile vasculitis, Kawasaki disease, Lichen planus, Mast Cell Activation Syndrome, Mastocytosis, Mixed connective tissue disease, Myositis, Osteoarthritis, Otitis, Pelvic inflammatory disease, Peripheral ulcerative keratitis, Pneumonia, Polymyositis, Psoriasis, Psoriatic arthritis, Reactive arthritis, Reperfusion injury, Rheumatic fever, Rheumatoid arthritis, Rhinitis, Sarcoidosis, Scleroderma, Sjogren's syndrome, Spondyloarthritis, Systemic juvenile idiopathic arthritis, Systemic lupus erythematosus, Systemic sclerosis, Transplant rejection, Type 1 diabetes mellitus, Type 2 diabetes mellitus, Undifferentiated connective tissue disease, Uveitis, and Vasculitis, and other autoimmune diseases associated with acute or chronic inflammation.

It is an object of the invention to provide a method of treatment or prevention of a condition or disease or at least one symptom associated therewith in a subject in need thereof as above, wherein the condition or disease is an autoimmune condition.

It is an object of the invention to provide a method of treatment or prevention of a condition or disease or at least one symptom associated therewith in a subject in need thereof as above, wherein the autoimmune condition comprises on or more of: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type 11, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

It is an object of the invention to provide a method of treatment or prevention of a condition or disease or at least one symptom associated therewith in a subject in need thereof as above, wherein the condition or disease is an infectious condition.

It is an object of the invention to provide a method of treatment or prevention of a condition or disease or at least one symptom associated therewith in a subject in need thereof as above, wherein the infectious condition is an infection comprising Helicobacter pylori, Borrelia burgdorferi, Legionella pneumophilia, Mycobacteria sp (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, Actinomyces israeli, and/or Chlamydia trachomatis.

It is an object of the invention to provide a method of treatment or prevention of a condition or disease or at least one symptom associated therewith in a subject in need thereof as above, wherein the infectious condition is an infection comprising Retroviridae (for example, HIV), Picornaviridae (for example, polio viruses, hepatitis A virus; enteroviruses, human coxsackie viruses, rhinoviruses, echoviruses), Caliciviridae (such as strains that cause gastroenteritis), Togaviridae (for example, equine encephalitis viruses, rubella viruses); Flaviridae (for example, dengue viruses, encephalitis viruses, yellow fever viruses), Coronaviridae (for example, coronaviruses); Rhabdoviridae (for example, vesicular stomatitis viruses, rabies viruses); Filoviridae (for example, ebola viruses), Paramyxoviridae (for example, parainfluenza viruses, mumps virus, measles virus, respiratory syncytial virus), Orthomyxoviridae (for example, influenza viruses), Bunyaviridae (for example, Hantaan viruses, bunya viruses, phleboviruses and Nairo viruses), Arenaviridae (hemorrhagic fever viruses), Reoviridae (e.g., reoviruses, orbiviurses and rotaviruses); Birnaviridae; Hepadnaviridae (Hepatitis B virus) Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses), Epstein-Barr virus, Poxviridae (variola viruses, vaccinia viruses, pox viruses), and Asfarviridae (such as African swine fever virus), and/or Norovirus.

It is an object of the invention to provide a method of treatment or prevention of a condition or disease or at least one symptom associated therewith in a subject in need thereof as above, wherein the infectious condition is an infection comprising Aspergillus sp., Candida albicans, Cryptococcus, Histoplasma, Cryptococcus neoformans, Histoplasma capsulatum, Coccidioides immitis, and/or Blastomyces dermatitidis.

It is an object of the invention to provide a method of treatment or prevention of a condition or disease or at least one symptom associated therewith in a subject in need thereof as above, wherein the infectious condition is an infection comprising Plasmodium falciparum or Toxoplasma gondii.

It is an object of the invention to provide a method of treatment or prevention of a condition or disease or at least one symptom associated therewith in a subject in need thereof as above, wherein the at least one inflammatory condition or disease is an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS.

### DESCRIPTION OF THE FIGURES

FIG. 1 schematically illustrates the inventive strategy of manipulating T cell differentiation by modulating the Glucose metabolism pathway of T cells by modulating (increasing) the expression of Pdk1/Pdp1. As shown, T cells rely on glucose for proliferation and differentiation and the glucose metabolic pathway diverges into two different directions at pyruvate. Pyruvate, when it is transported into mitochondria, is converted to acetyl CoA by pyruvate dehydrogenase (PDH), then it can be utilized as a carbon source for the TCA cycle, leading to the synthesis of large amounts of ATP (mitochondrial oxidation). Pyruvate can also be converted into lactate as the final stage of glycolysis, requiring no oxygen consumption, but yielding a much lower amount of ATP. Pyruvate dehydrogenase kinase (PDK) and Pyruvate dehydrogenase phosphatase (PDP) regulate the phosphorylation of PDH enzyme, which inactivates its ability to convert pyruvate into acetyl CoA for use in the TCA cycle. Memory T cells rely on mitochondrial oxidation while effector T cells utilize glycolysis. Therefore, increased expression of PDH regulatory enzymes (PDK1 and PDP1) in T cells should promote T cell differentiation and improve T cell-mediated therapy.

FIGS. 2A and B show the metabolic profiles of engineered CD8+ T cells according to the invention. FIG. 2A contains the results of a glycolytic rate assay and FIG. 2B contains mitostress assay results of Pdk1/Pdp1 overexpression measured by a Seahorse instrument indicating the engineered cells showed enhanced metabolic capacity. In these experiments the cells were then activated with anti-CD3 and anti-CD28 antibodies, and transduced with empty retroviral vector (EV), Pdk1, or Pdp1 encoding retroviruses

FIGS. 3A-3D. shows the results of experiments wherein effector and memory populations of engineered OT-1 T cells after antigenic challenge CD45.1⁺ OT-1 T cell were isolated from CD45.1+ OT-I⁺ mouse splenocytes. In these experiments the cells were then activated with anti-CD3 and anti-CD28 antibodies, and transduced with empty retroviral vector (EV), Pdk1, or Pdp1 encoding retroviruses. The transduced cells were adoptively transferred into mice infected by with murine gammaherpesvirus-68 expressing ova (MHV-68-ova) (FIG. 3A-C) or Listeria monocytogenes expressing ova (FIG. 3D). CD45.1⁺ OT-I cells from spleens, livers, and lungs were analyzed using flow cytometry (FIG. 3A, B) 10 (effector) and (FIG. 3C) 50 (memory) days after the transfer. * p<0.05, ** p<0.01, *** p<0.001 by Student's t-test.

FIG. 4. contains experiments showing the enhanced memory precursor differentiation of engineered cells according to the invention. In these experiments memory precursor effector cells (MPEC) and short-lived effector cells (SLEC) were measured in Pdk1/Pdp1-transduced cells at 10 days after MHV-68-ova infection in mice. * p<0.05, ** p<0.01, *** p<0.001 by Student's t-test.

FIGS. 5A-SC. contains experimental results showing that engineered cells according to the invention showed greater proliferation and less cell death. In these experiments Pdk1/Pdp1 or EV-transduced cells at 10 days after MHV-68-ova infection in mice were analyzed for selected phenotypic markers of proliferation (Ki-67*) and apoptosis (annexin V⁺ 7-AAD-). (FIG. 5A) Cells from the spleen were stained with anti-Ki-67 antibody. (FIG. 5B-C) Cells from spleen (FIG. 5B) and lungs (FIG. C) were stained with fluorescently labeled annexin-V and 7-AAD. * p<0.05, ** p<0.01, *** p<0.001 by Student's t-test.

FIG. 6. contains experimental results showing that engineered cells according to the invention showed increased effector molecule production. In these experiments Pdk1/Pdp1 or EV-transduced cells at 10 days after MHV-68-ova infection in mice were analyzed by flow cytometry for effector molecule content. Cells were stimulated by SIINFEKL peptide for 5 hours with Brefeldin A to prevent the secretion of cytokines. Granzyme B, TNFα, and Interferon γ expression were analyzed in splenocytes and cells from lungs. * p<0.05, ** p<0.01, *** p<0.001 by Student's t-test.

FIG. 7. contains experimental results showing that engineered cells according to the invention showed enhanced memory population and tissue resident memory (T_{RM}) subset formation. In these experiments Pdk1/Pdp1 or EV-transduced cells were analyzed by flow cytometry at 50 days after MHV-68-ova infection in mice. Mice were given anti-CD8β-PE antibody intravenously via tail vein injection before euthanasia to stain cells in the circulation. Lung-infiltrating cells negative for CD8β-PE staining were gated and analyzed. CD45.1+ OT-1 cell population along with T_{RM} phenotype markers (CD62L⁻ CD103+CD69+) were measured by flow cytometry. * p<0.05, ** p<0.01, *** p<0.001 by Student's t-test.

FIG. 8A-8D. contains experimental results showing that engineered cells according to the invention showed improved recall response and functional characterization of the engineered cells after secondary antigenic challenge. Adoptively transferred Pdk1/Pdp1 or EV-transduced cells were harvested from spleens of the recipient mice at 30 or 51 days after MHV-68-ova infection. The CD45.1⁺ OT-1 memory cells were magnetically purified and were transferred again into new recipient mice intraperitoneally infected by MHV-68-ova. (A) OT-1 memory cells were analyzed from spleens at 5 or 7 days after secondary transfer. (B) Spleen cells at 5 days post-secondary antigenic challenge were stained for Ki-67 to measure proliferation and (C) for 7-AAD and Annexin V for cell death/apoptosis. (D) Cells from spleen at 5 days post-secondary antigenic challenge were stimulated by SIINFEKL peptide for 5 hours with Brefeldin A to prevent the secretion of cytokines. Granzyme B, TNFα, and Interferon γ were analyzed in splenocytes. * p<0.05, ** p<0.01, *** p<0.001 by Student's t-test.

FIG. 9. contains a schematic of the parental retroviral vector used to derive the retroviral vectors used in the examples to obtain exemplary genetically engineered T cells according to the invention. As disclosed infra, the coding sequences for Pdp1 or Pdk1 were cloned into parental vector pCl-mCD19.

FIG. 10. contains a schematic of retroviral vector pCI-mCD19-PDP1. As shown the DNA sequence encoding Pdp1 from mouse (NCBI Reference Sequence: NM_001290387.1) was cloned into pCI-mCD19 to produce pCI-mCD19-PDP1 and the resultant plasmid was used to produce retroviral particles for transduction.

FIG. 11. contains a schematic of retroviral vector pCl-mCD19-PDK1. As shown the DNA sequence encoding Pdk1 from mouse (GenBank: BC027196.1) was cloned into pCI-mCD19 to produce pCl-mCD19-PDK1. This plasmid was used to produce retroviral particles for transduction.

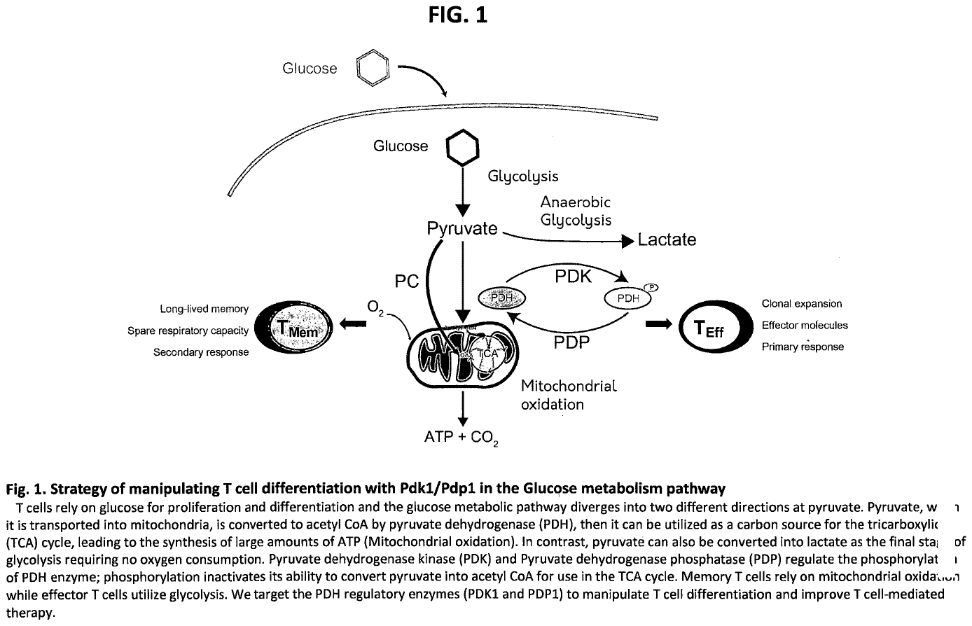

### DETAILED DESCRIPTION

Provided herein are methods for producing T cells, e.g., human T cells, which are engineered to upregulate mitochondrial and/or glycolytic metabolism, engineered T cells produced by such methods and compositions containing, and their use in adoptive cell therapies, for example treatment of cancer, particularly solid tumors, tumor reoccurrence and/or metastasized tumors, infectious conditions, and chronic inflammatory or autoimmune conditions. These engineered T cells possess advantages which render them well suited for usage in adoptive T therapies including e.g., increased survival, increased effector functions, and enhanced secondary response of memory CD8 T cells upon antigen re-exposure.

In particular, the present invention generally relates to isolated CD8+ T cells which are engineered to stably upregulate mitochondrial and/or glycolytic metabolism by introducing genetic constructs which provide for stable and heritable expression of PDP and/or PDK. The present invention also relates to methods for producing said cells.

The present invention also relates to methods for the use of these modified T cells which are engineered to stably upregulate mitochondrial and/or glycolytic metabolism in cell therapy, for example treatment of cancer, particularly solid tumors, infectious conditions, and chronic inflammatory and autoimmune conditions.

As noted above, the present invention solves the problem of T cells relying on inefficient anaerobic glycolysis in the tumor microenvironment and thereby losing their competition for glucose (which is metabolized by rapidly proliferating tumor cells) by providing T cells having enhanced mitochondrial and/or glycolytic activity. As exhausted T cells have less functional mitochondria, augmenting mitochondrial and/or glycolytic metabolism should alleviate mitochondrial and/or glycolytic dysfunction, and thereby reduceTcell exhaustion at diseased sites e.g., in solid tumors or sites of inflammation, autoimmunity, or infection thereby enhancing the efficacy of adoptive T cell-mediated therapies.

In particular, the present invention provides genetically engineered T cells, preferably primary human T cells, and more preferably primary human CD8+ and/or CD4+ T cells which are genetically modified in order to upregulate mitochondrial and/or glycolytic metabolism by increasing the flux of pyruvate into the citric acid cycle and thereby increasing mitochondrial and/or glycolytic metabolism. In general, these comprise cells are genetic modified to provide stable, heritable expression of pyruvate dehydrogenase kinase enzyme (PDK) and/or pyruvate dehydrogenase phosphatase (PDP) enzyme in said cells.

Further the present invention provides methods for generating such genetically engineered T cells, preferably primary human T cells, and more preferably primary human CD8+ and/or CD4+ T cells which are genetically modified to provide stable, heritable expression of pyruvate dehydrogenase kinase enzyme (PDK) and/or pyruvate dehydrogenase phosphatase (PDP) enzyme.

Also, the present invention pharmaceutical compositions comprising genetically engineered T cells, preferably primary human T cells, and more preferably primary human CD8+ and/or CD4+ T cells which are genetically modified to provide stable, heritable expression of pyruvate dehydrogenase kinase enzyme (PDK) and/or pyruvate dehydrogenase phosphatase (PDP) enzyme.

Additionally, the invention provides methods of therapy comprising the administration of genetically engineered T cells, preferably primary human T cells, and more preferably primary human CD8+ and/or CD4+ T cells, and pharmaceutical compositions comprising same wherein said Tcells are genetically modified to provide stable, heritable expression of pyruvate dehydrogenase kinase enzyme (PDK) and/or pyruvate dehydrogenase phosphatase (PDP) enzyme.

In particular the present invention provides three methods for genetically modifying T cells in order to upregulate mitochondrial and/or glycolytic metabolism therein by increasing the flux of pyruvate into the citric acid cycle and thereby increasing mitochondrial and/or glycolytic metabolism. These three methods all involve introducing at least one genetic construct into T cells, preferably primary human T cells, and more preferably primary human CD8+ and/or CD4+ T cells, as follows:
(iv) introducing at least one genetic construct which provides for the stable, heritable expression of pyruvate dehydrogenase kinase enzyme (PDK);
(v) introducing at least one genetic construct which provides for the stable, heritable expression of pyruvate dehydrogenase phosphatase (PDP) enzyme; or
(vi) introducing at least one genetic construct which provides for the stable, heritable expression of pyruvate dehydrogenase kinase enzyme (PDK) and pyruvate dehydrogenase phosphatase (PDP) enzyme.

The above methods may further comprise introducing at least one other modification which provides for the expression or the absence of and/or impaired expression of one or more other gene products. For example engineered T cells which are genetically modified according to the invention may be further engineered such that they express an exogenous receptor, e.g., a chimeric antigen receptor or a polypeptide which elicits a desired functional or therapeutic effect such as a hormone, cytokine, a polypeptide which promotes or reduces the expression or activity of a stimulatory immune checkpoint molecule such as CD27, CD28, CD40, CD122, CD137, OX40, GITR or ICOS and/or a polypeptide which promotes or reduces the expression or activity of an inhibitory immune checkpoint molecule such as A2AR, A2BR, B7-H3, B7-H4, BTLA, CTLA-4, IDO, KIR, LAG3, NOX2, PD-1, PD-L1, VISTA or SIGLEC7). Also, engineered T cells which are genetically modified according to the invention may be further genetically modified such that they comprise impaired or no expression of an endogenous polypeptide, e.g., the endogenous T cell receptor (TCR) and/or endogenous an HLA receptor, e.g., HLA Class I, or HLA Class II receptor, thereby permitting the cells to be used in allogeneic donors.

In exemplary embodiments genetically engineered T cells according to the invention are obtained using retroviral constructs which upon introduction into T cells, preferably primary human T cells, and more preferably primary human CD8+ and/or CD4+ T cells, provide for retrovirally-mediated expression of pyruvate dehydrogenase kinase enzyme and/or retrovirally mediated expression of pyruvate dehydrogenase phosphatase enzyme in such T cells. As described in detail infra, these methods have been demonstrated to result in increased mitochondrial and/or glycolytic metabolism in such T cells, and in a murine infectious disease model these T cells have been demonstrated to provide for increased numbers of T cells surviving in tissues, increased T effector functions and enhanced secondary responses of memory CD8 T cells upon antigen re-exposure.

Based on the increased activity and effector functions of T cells transduced with these genes, which optionally may be further engineered with other constructs which provide for the expression or increased expression of a desired polypeptide, and/or the impaired expression of a particular polypeptide, when used in adoptive T cell therapies, e.g., for treating cancer, infectious conditions, inflammatory conditions and/or autoimmune conditions in subjects in need thereof should lead to enhanced and more prolonged potency. This moreover should facilitate the use of engineered T cells according to the invention for treatment of conditions which are often recalcitrant to adoptive T cell therapies, e.g., treatment of solid tumors and/or cancers which have relapsed and/or metastasized.

While retroviral vectors are used in the working examples to produce engineered T cells according to the invention, it is envisioned that other techniques and vectors useful for modifying human cells, particularly primary human cells and more particularly human T cells may alternatively be used. Different types of viral vectors may be used to produce engineered T cells according to the invention, e.g., retroviral vectors (e.g., MMSV, MSCV), lentiviral vectors (HIV01, HIV-2), adenoviral vectors (e.g., Ad5 vectors), alphavirus vectors (e.g., SFV, SIN, VEE, M1 vectors), herpes simplex vectors (e.g., HSV1, HSV vectors), and adeno-associated vectors (AAV) (e.g., AAV2, 3, 5, 6, 8, 9 vectors).

Also, genetically engineered T cells according to the invention may be obtained by use of gene editing, e.g., for example, a CRISPR/Cas-based genetic engineering method, a TALEN-based genetic engineering method, a zinc finger (ZF)-nuclease genetic engineering method, and transposon-based genetic engineering methods.

As shown herein the inventive methods provide for increased mitochondrial and/or glycolytic metabolism in T cells and have been shown in vivo in a disease model to possess significant advantages including enhanced T cell survival including survival in diseased tissues, increased T effector functions and elicit enhanced secondary response of memory CD8 T cells upon antigen re-exposure. Based on these advantages these engineered T cells when used in adoptive T cell therapies should provide for enhanced therapeutic efficacy, e.g., enhanced anti-tumor efficacy, against both hematologic and solid tumors, enhanced ability to prevent cancer reoccurrence, and enhanced efficacy in the treatment of other conditions where T cells are currently used for adoptive T cell therapy such as in the treatment of infectious diseases, inflammatory and autoimmune conditions and neurodegenerative conditions.

In some embodiments the invention provides methods for producing T cells having increased mitochondrial and/or glycolytic metabolic activity. In some embodiments, the invention provides T cells with increased proliferation and greater resistance to death and apoptosis. In some embodiments, the invention provides T cells with enhanced effector functions.

In other embodiments, the invention provides methods for treating cancer, infectious disease, inflammatory disease, and/or autoimmune disease by administering an effective amount of the engineered T cells of the invention. In embodiments, the T cells of the invention are administered before, along with, or after the administration of other therapies. In embodiments, the said other therapies comprise chemotherapeutic agents, antibiotics, therapeutic antibodies, cytokines, and/or cell therapy.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

### Definitions

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this invention belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “treatment” (and grammatical variations thereof such as “treat” or “treating”) or “prevention” (and grammatical variations thereof such as “prevent” or “preventing”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result alone or in combination with other active agents.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. Generally, the response is either amelioration of symptoms in a patient or a desired biological outcome (e.g., reduction of tumor size, infection, inflammation, infection and the like). The therapeutically effective amount may vary according to factors such as the disease state, age, sex, and weight of the subject. In some embodiments, the provided methods involve administering the compositions at effective amounts, e.g., therapeutically effective amounts alone or in combination with other active agents or therapies.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower disease burden, the prophylactically effective amount in some aspects will be higher than the therapeutically effective amount.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid, vitamin A, vitamin E, and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, cysteine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG); retinyl palmitate, selenium, methionine, citric acid, sodium sulfate and parabens Examples of diluent include, but are not limited to, water, alcohol, saline solution, glycol, mineral oil and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation. The pharmaceutical composition may further contain additional pharmaceutical or therapeutic agent, as evaluated beneficial by the physician administering said pharmaceutical composition.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and non-human primates (including monkeys, chimpanzees, orangutans and gorillas) are included within the definition of subject. In some embodiments, the subject has been treated with one or more additional cancer therapies prior to the administration of the modified cells. In some aspects, the subject may be or may have become refractory or non-responsive to the other treatment. In some embodiments, the subject may not have become refractory or non-responsive but the administration of the modified cells is carried out to complement the other treatment and/or enhance the subject's response to the other treatment. In some embodiments the modified cells are administered prior to or simultaneously with the other treatment. It is contemplated by this disclosure that the other treatment comprising one or more additional cancer therapies may include immunotherapy, chemotherapy, targeted therapy, stem cell transplant, radiation, surgery, and/or hormone therapy. In some embodiments, the immunotherapy may include immune checkpoint inhibitors (e.g., negative checkpoint blockade), monoclonal antibodies, cancer vaccines, immune system modulators, and/or adoptive cell therapies such as CAR-T cell therapy, exogenous TCR T cell therapy, and TIL therapy.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, intrapleural, oral, sublingual buccal, rectal, vaginal, nasal ocular administrations, as well as infusion, inhalation, and nebulization. The phrases “parenteral administration” and “administered parenterally” as used herein means modes of administration other than enteral and topical administration.

As used herein, the terms “engineer” and “engineered” should be understood to refer to genetic engineering of cells, in that the DNA content of the cells is modified.

As used herein, “cancer” refers to any disease in which abnormal cells divide without control and which can invade nearby tissues or spread to other parts of the body through the blood and lymph systems. Cancer may include carcinomas (cancers that begin in the skin or in tissues that line or cover internal organs), sarcomas (cancers that begin in bone, cartilage, fat, muscle, blood vessels, or other connective or supportive tissue), leukemias (cancers that start in blood-forming tissue, such as the bone marrow, and causes large numbers of abnormal blood cells to be produced and enter the blood), lymphomas and multiple myelomas (cancers that begin in the cells of the immune system), and central nervous system cancers (cancers that begin in the tissues of the brain and spinal cord). Cancer may also refer to any malignancy. Types of cancer include but are not limited to adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

As used herein, “PDP” and other forms thereof (including “Pdp1” and “PDP1”) refers to “pyruvate dehydrogenase phosphatase” protein, transcript (mRNA), and/or gene expressing said protein from human (Gene ID No. 54704), mouse (Gene ID No. 381511), or from any other mammalian species, including all isoforms thereof. PDP is also known as Protein Phosphatase 2C Catalytic Unit, PPM2A, PPM2C, PDPC 1, and PDPC. PDP may have a cDNA nucleotide sequence which is at least 75% identical, at least 80% identical, at least 85% identical, at least 90% identical, at least 95% identical, at least 98% identical, at least 99% identical or more to SEQ ID NO: 4 or SEQ ID NO: 6 or to any other mammalian PDP cDNA sequence. PDP may have an amino acid sequence which is at least 75% identical, at least 80% identical, at least 85% identical, at least 90% identical, at least 95% identical, at least 98% identical, at least 99% identical or more to SEQ ID NO: 8 or SEQ ID NO: 10 or to any other mammalian PDP amino acid sequence.

As used herein, “PDK” and other forms thereof (including “Pdk1” and “PDK1”) refers to “pyruvate dehydrogenase kinase” protein, transcript (mRNA), and/or gene expressing said protein from human (Gene ID No. 5163), mouse (Gene ID No. 228026), or from any other mammalian species, including all isoforms thereof. PDP is also known as PDH Kinase 1, PDHK1, Pyruvate Dehydrogenase Kinase, Isoenzyme 1, and Pyruvate Dehydrogenase (Acetyl-Transferring) Kinase Isozyme 1, Mitochondrial. PDK may have a cDNA nucleotide sequence which is at least 75% identical, at least 80% identical, at least 85% identical, at least 90% identical, at least 95% identical, at least 98% identical, at least 99% identical or more to SEQ ID NO: 5 or SEQ ID NO: 7 or to any other mammalian PDK cDNA sequence. PDK may have an amino acid sequence which is at least 75% identical, at least 80% identical, at least 85% identical, at least 90% identical, at least 95% identical, at least 98% identical, at least 99% identical or more to SEQ ID NO: 9 or SEQ ID NO: 11 or to any other mammalian PDP amino acid sequence.

As used herein, the terms “mitochondrial metabolism”, “mitochondrial oxidation”, “mitochondrial activity”, and “oxidative phosphorylation” refer to all the energy producing reactions that occur in mitochondria that comprise the tricarboxylic acid cycle and oxidative phosphorylation. Increases in mitochondrial metabolism can be observed, e.g., by increases in parameters such as a cellular oxygen consumption rate (OCR) or mitochondrial mass. Increases in mitochondrial metabolism may also be associated with increases in functional activity of the cell, such as CD8+ T cell effector activity.

As used herein the term “glycolytic metabolism” or “glycolysis” refers the metabolic pathway that converts glucose (C₆H₁₂O₆) into pyruvate (CH₃COCO₂H). Glycolysis is a metabolic pathway that does not require oxygen (i.e., in anaerobic conditions pyruvate is converted to lactic acid). The free energy released in this process is used to form high-energy molecules such as adenosine triphosphate (ATP) and reduced nicotinamide adenine dinucleotide (NADH). Glycolysis involves numerous reactions which are catalyzed by enzymes. In most organisms, glycolysis occurs in the cytosol. The most common type of glycolysis is the Embden-Meyerhof-Parnas (EMP) pathway, which was discovered by Gustav Embden, Otto Meyerhof, and Jakub Karol Parnas. Glycolysis also includes other pathways, such as the Entner-Doudoroff pathway and various heterofermentative and homofermentative pathways. The glycolysis pathway can be separated into two phases, (i) Investment phase—wherein ATP is consumed; and the (ii) Yield phase—wherein more ATP is produced than originally consumed. Generally when we refer to glycolysis or glycolytic metabolism we refer to glycolysis reactions that occur in T cells, typically human T cells which are engineered to express increased amounts of PDK and/or PDP.

As used herein, the term “T cell” includes any cell which endogenously expresses a TCR on its cell surface or which upon maturation or differentiation gives rise to a TCR on its cell surface. These cells are one of the important white blood cells of the immune system and play a central role in the adaptive immune response. T cells are derived from hematopoietic stem cells, found in the bone marrow. T cell subtypes have a variety of important functions in controlling and shaping the immune response. One of these functions is immune-mediated cell death, and it is carried out by two major subtypes: CD8+“killer” and CD4+“helper” T cells. (These are named for the presence of the cell surface proteins CD8 or CD4.) CD8+ T cells, also known as “killerT cells”, are cytotoxic—this means that they are able to directly kill virus-infected cells, as well as cancer cells. CD8+ T cells are also able to use small signaling proteins, known as cytokines, to recruit other types of cells when mounting an immune response. A different population of T cells, the CD4+ T cells, function as “helper cells”. Unlike CD8+ killerT cells, the CD4+ helper T (TH) cells function by further activating memory B cells and cytotoxic T cells, which leads to a larger immune response. The specific adaptive immune response regulated by the TH cell depends on its subtype, which is distinguished by the types of cytokines they secrete. In the present application a T cell includes by way of example a recombinant or isolated cell e.g., a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cell, and a/b T cell, a g/d T cell, and a Natural KillerT (NKT) cell. In preferred embodiments the T cells used in the inventive methods comprise human T cells, preferably primary human T cells and more preferably primary human CD4+ or CD8+ T cells.

As used herein, the term “autologous” refers to any material derived from the same individual to which it is later to be re-introduced. In some embodiments, the engineered autologous T cell therapy method described herein involves collection of lymphocytes from a patient, which are then engineered to express, e.g., a PDP or PDK transgene, and then administered back to the same patient.

As used herein, the term “allogeneic” refers to any material derived from one individual which is then introduced, optionally after engineering according to the methods described herein, to another individual of the same species, e.g., allogeneic engineered T cell transfer.

As used herein, the terms “encode”, “encodes”, or “encoding” refers a nucleotide sequence which contains the information necessary to specify the amino acid sequence of a protein.

As used herein, the term “coding region” of a gene consists of the nucleotide residues of gene that encode the protein itself, exclusive of other regions of the gene such as a 5′ untranslated region, 3′ untranslated region, etc.

As used herein, the term “gene” consists of the residues of a nucleic acid that encode the protein or RNA that is intended to be produced in addition to other nucleic acid sequences such as promoters, enhancers, introns, 5′ untranslated regions, ribosome binding site(s), 3′ untranslated regions, polyadenylation signal(s), and other accessory sequences necessary for efficient expression.

As used herein, the term “vector” refers to any plasmid or virus comprising an exogenous nucleic acid which is suitable for conveying a nucleic acid from outside of a cell into the interior of said cell. The term should also be construed to comprise non-plasmid and non-viral compounds which facilitate transfer of nucleic acid into cells, including, for example, polylysine compounds, cationic lipids, and the like. The vector may be a viral vector which is suitable as a delivery vehicle for delivery of a nucleic acid or the vector may be a non-viral vector which is suitable for the same purpose. Viral vectors comprise retroviral vectors and lentiviral vectors. The vector may also comprise a transposon system wherein the transposon system facilitates the transfer of a gene from an exogenous DNA into the host cell chromosome. Genes carried by vectors may also comprise promoters, enhancers, introns, 5′ untranslated regions, ribosome binding site(s), 3′ untranslated regions, polyadenylation signal(s), and other accessory sequences necessary for efficient expression. Vectors may comprise additional nucleic acids other than that comprising the transgene, such as nucleic acids encoding a transposase. The vector may also consist of DNA, which may be introduced to the cells by physical methods such as electroporation or microinjection.

As used herein, the term “cytokine,” refers to a protein that is released by one cell in response to a stimulus, wherein the cytokine interacts with an immune cell to mediate a response in the immune cell. A cytokine can be endogenously expressed by a cell or administered to a subject. Cytokines may be released by immune cells, including macrophages. B cells, T cells, and mast cells to propagate an immune response. Cytokines can induce various responses in the recipient cell. Cytokines can include or exclude homeostatic cytokines, chemokines, pro-inflammatory cytokines, effectors, and acute-phase proteins. Homeostatic cytokines, including interleukin (IL) 7 and IL-15, promote immune cell survival and proliferation, and pro-inflammatory cytokines can promote an inflammatory response. Examples of homeostatic cytokines can include or exclude, IL-2, IL-4, IL-5, IL-7, IL-10, IL-12p40, IL-12p70, IL-15, and interferon (IFN) gamma. Examples of pro-inflammatory cytokines can include or exclude, IL-1a, IL-1b, IL-6, IL-13, IL-17a, tumor necrosis factor (TNF)-alpha TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors can include or exclude, granzyme A, granzyme B, soluble Fas ligand (sFasL), and perforin. In some embodiments, acute phase-proteins can include or exclude C-reactive protein (CRP) and serum amyloid A (SAA). In some embodiments, cytokines of this disclosure can include or exclude: chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), granulocyte macrophage colony-stimulating factor (GM-CSF), leukemia inhibitory factor (LIF), oncostatin M (OSM), CD154, lymphotoxin (LT) beta, 4-1BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

As used herein, the term “chemokine” refers to a type of cytokine that mediates cell chemotaxis, or directional movement. Examples of chemokines can include or exclude, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1a, MIP-1a), MIP-1(3 (MIP-1b), gamma-induced protein 10 (IP-10), and thymus and activation regulated chemokine (TARC or CCL17).

As used herein, the terms “chimeric antigen receptor” or “CAR” refer to a fusion protein comprising antigen recognition moieties and cell-activation elements. In some embodiments, the CAR comprises an extracellular domain, a transmembrane domain, and an intracellular domain, each of which are polypeptides encoded by a corresponding polynucleotide sequence. In certain embodiments, the CAR is designed to have two, three, four, or more costimulatory domains. Exemplary antigen receptors and methods for engineering and introducing such receptors into cells, include those described, for example, in international patent application publication numbers W0200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Morgan et al., 2006, Science 314:126-129; Johnson et al., 2009, Blood 114:535-546; Robbins, et al., 2011, J Clin Oncol 29:917-924; Rapaport, et al., 2015, Nat Med 21:914-921; Neelapu et al., 2017, N Engl J Med 377:2531-2544; Maude et al., 2018, N Engl J Med 378:439-448; Davila et al., 2014, Sci Transl Med 6:224ra25; Maude et al., 2014, N Eng/J Med 371:1507-1517; Kochenderfer, et al., 2015, J Clin Oncol 33:540-549; Porter et al., 2015, Sci Transl Med 7:303ra139; Turtle et al., 2017, J Clin Oncol 35:3010-3020; Brudno et al., 2018, J Clin Oncol 36(22):2267-2280, Sadelain et al., 2013, Cancer Discov. 3(4):388-398; Davila et al., 2013, PLoS ONE 8(4):e61338; Turtle et aL., 2012, Curr. Opin. Immunol., 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276; Wang et al., 2012, J. Immunother. 35(9): 689-701; and Brentjens et al., 2013, Sci Transl Med. 2013 5(177). See also International Patent Publication No.: WO2014031687, U.S. Pat. Nos. 8,339,645, 7,446,179, 7,446,190, and 8,389,282, and U.S. patent application Publication No. US 2013/0149337.

As used herein, the terms “CAR T cell” or “CAR T lymphocyte” refer to a T cell containing or capable of producing a CAR polypeptide, regardless of actual expression level. A cell that is capable of expressing a CAR is a T cell containing nucleic acid sequences for the expression of the CAR in the cell.

As used herein, the terms “exogenous T cell receptor” and “exogenous TCR” refer to a TCR with specific α and β chains. As used herein, an exogenous TCR recognizes and binds to a specific peptide or set of peptides when said peptides are bound by and displayed by a particular MHC molecule or set of MHC molecules. Cells expressing an exogenous TCR may also be engineered to express exogenous CD3 gamma, delta, epsilon and zeta genes to enhance expression of the exogenous TCR.

As used herein, the term “antigen” refers to any molecule that provokes an immune response or is capable of being bound by an antibody. The immune response may involve either antibody production, or the activation of specific immunologically competent cells, or both. A person of skill in the art would readily understand that any macromolecule, including virtually all proteins or peptides, can serve as an antigen. An antigen can be endogenously expressed, i.e. expressed by genomic DNA, or can be recombinantly expressed. An antigen can be specific to a certain tissue, including a cancer cell, or it can be broadly expressed. In addition, fragments of larger molecules can act as antigens. In one embodiment, antigens are tumor-associated antigens.

As used herein, the term “tumor-associated antigen” refers to proteins, glycoproteins or carbohydrates that are specifically or preferentially expressed by cancer cells as compared to non-cancerous cells. Tumor-associated antigens include, e.g., Alphafetoprotein, BCMA, B7-H3, Carcinoembryonic antigen, CA-135, CD19, CD133, Claudin 18.2, c-Met, EGFR, FAP, GD2, GPC3, HER-2, MAGE, Mesothelin, MUC-1, MUC-16, NY-ESO-1, PD-L1, PSCA, PSMA, ROR1, and Tyrosinase.

As used herein, the term “immunostimulatory agent” refers to proteins, including antibodies, cytokines, small molecules, carbohydrates, lipids, oligonucleotides, antigens, microbes and/or their components, and any combination thereof which have the effect of increasing the activity or efficacy of the immune response in a subject. As such, immunostimulatory agents comprise antagonist antibodies to immune checkpoint proteins such as PD-1 or PD-L1, cytokines such as IL-2, small molecules (see, e.g., U.S. Pat. No. 10,004,755B2), compounds such as Quillaja saponins (see, e.g., U.S. Pat. No. 10,729,764B2), mycobacterial lipids (see, e.g., U.S. Pat. No. 8,563,009B2), oligonucleotides such as CpG containing oligonucleotides (see e.g., U.S. Pat. No. 8,232,259B2), antigens such as prostatic acid phosphatase, and compositions comprising such compounds such as a Bacillus Calmette-Guerin vaccine.

1. Methods and Vectors for Producing T Cells Having Upregulated Mitochondrial and/or Glycolytic Metabolism According to the Invention

The present disclosure provides different vectors and methods of use thereof for obtaining T cells wherein mitochondrial and/or glycolytic metabolism is upregulated. In general, these methods comprise introducing gene constructs, e.g., viral gene constructs or gene editing constructs which provide for stable, heritable expression of pyruvate dehydrogenase kinase enzyme (PDK) and/or pyruvate dehydrogenase phosphatase (PDP) enzyme in T cells, preferably primary human T cells and more preferably primary human CD8+ and/or CD4+ T cells.

The present disclosure provides vectors or constructs including plasmids and viral constructs suitable for expressing the transgenes of the disclosure in T cells. A nucleotide sequence such as a one encoding an pyruvate dehydrogenase kinase enzyme (PDK) and/or pyruvate dehydrogenase phosphatase (PDP) enzyme or optionally a CAR or other desired coding sequence may be inserted into a vector or viral construct, including those from retroviruses, lentiviruses, adenoviruses, and adeno-associated viruses (AAV). Viral vector technology is well known in the art and is described, for example, in Sambrook et al. (2001, Molecular Cloning: A Laboratory Manual, Cold Spring Harbor Laboratory, New York), and in other virology and molecular biology manuals. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. The expression of natural or synthetic nucleic acids encoding proteins, mRNA, or non-coding RNAs of interest may typically be achieved by operably linking a nucleic acid encoding said proteins, mRNA, or non-coding RNAs to a promoter, and incorporating the construct into an expression vector. The vectors can be suitable for replication or replication and integration in eukaryotes. Typical vectors contain transcription and translation terminators, initiation sequences, and promoters (either constitutive or inducible promoters) useful for regulation of the expression of the desired nucleic acid sequence. In general, a suitable vector contains an origin of replication functional in at least one organism, a promoter sequence, convenient restriction endonuclease sites, and one or more selectable markers. In some embodiments, the method for engineering the T cells may comprise other transgenes, for example transgenes encoding cytokines, CARs, or exogenous TCRs. In a preferred embodiment, the transduction of said genes is accomplished with a retroviral vector.

In general, engineered Tcells according to the invention may be obtained by inserting a DNA sequence which encodes either a PDP1 gene or a PDK1 into a viral vector, contacting the viral vector with a T cell to form or produce a virally-transduced T cell, and growing the viral-vector transduced T cells, and harvesting and/or purifying the viral vector-transduced cells. Types of viral vectors including retroviral vectors which may be used to obtain genetically modified T cells according to the invention are described briefly below.

a. Viral Vectors and Methods of Use for Producing Modified T Cells According to the Invention

The 5 main classes of viral vectors which are used for gene therapy can be categorized in 2 groups according to whether their genomes integrate into host cellular chromatin (oncoretroviruses and LV) or persist in the cell nucleus predominantly as extrachromosomal episomes (AAV, AV, and herpes viruses). The choice of viral vectors for creating cells for potential clinical use depends on the efficiency of transgene expression, ease of production, safety, toxicity, and stability. Also, the types of vectors are represented by both RNA and DNA viruses with either single-stranded (ss) or double-stranded (ds) genomes. The main groups of viral vectors used in gene therapy which potentially may be used to produce engineered T cells according to the invention are briefly disclosed below. 1. Retrovirus Vectors

As noted previously, in exemplary embodiments retroviral vectors are used to produce modified T cells according to the invention. Retroviruses are relatively complex enveloped RNA viruses with a diploid ssRNA genome and consist of at least 4 genes, gag, pro, pol, and env. The gag gene encodes the primary structural polyprotein, which is necessary for the assembly of noninfectious and immature viral-like particles. The pro gene encodes the viral protease and is responsible for facilitating the maturation of viral particles. Products of the pol gene include reverse transcriptase, RNase H, and integrase, whereas env is responsible for the viral surface glycoprotein and transmembrane proteins that mediate cellular receptor binding and membrane fusion.

Retroviruses may be broadly divided into two categories: namely, “simple” and “complex”. Retroviruses may even be further divided into seven groups. Five of these groups represent retroviruses with oncogenic potential. A review of these retroviruses is presented in Coffin et al. (1997) (ibid).

Retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. Integrase is encoded by the 3′ end of the pol gene, which also codes for two other viral enzymes, the protease and the reverse transcriptase. These three enzymes are initially synthesized as part of a larger polyprotein that is subsequently cleaved by the protease into the individual proteins.

The typical feature of RV and retroviral vectors is their ability to integrate into host DNA. Besides, complex RV such as HIV-1 encode accessory proteins that enhance replication and infectivity. Viral RNA is reversibly transcribed and integrated into the form of a provirus. The RV very effectively cooperate with enzymes of the host cell, which they use for their replication and long-term expression of viral proteins. Like most viruses, the entry of the RV into the host cell is receptor-dependent. Several of the oncogenic RV are replication-defective forms that have substituted a part of their standard viral gene complement with an oncogene sequence. Replication-competent retroviruses also cause malignant disease and a range of other pathogenic states in a wide variety of species. The acquired immunodeficiency syndrome (AIDS) is caused by the retroviruses HIV-1 and HIV-2.

Retroviral vectors have been extensively used to deliver therapeutic genes in the context of gene therapy, clinical applications for monogenic disorders, cancer, and infectious diseases, providing a stable and efficient expression of the transgene to patients. Retroviruses have numerous advantages over other vectors. The most significant advantage that retroviral vectors offer is their ability to transform their ssRNA genome into a dsDNA molecule that stably integrates into the target cell genome. This feature enables the retroviral vectors to modify the host cell nuclear genome permanently.

Recently, retroviral-vector-mediated gene transfer has involved the development of a new retroviral vectors class referred to as LVs. The LVs have the unique ability among RV to infect noncycling cells. Vectors derived from LV have been used in gene-editing technology and gene transfer in vivo. 2. Lentivirus Vectors

In alternative embodiments lentivirus vectors are potentially used to produce modified T cells according to the invention. Lentiviruses, a type of RV, consist of a single-stranded positive-sense RNA sequence that is transcribed into a DNA and integrated into the host genome, causing persistent infection. Most lentiviral vectors (LVVs) are derived from HIV-1 and retain the capability to integrate into the genome of infected cells. Wild-type HIV genome with all of its genes and regulatory elements provides the backbone for LVVs. In the first-generation LVVs, the envelope protein and the psi (L) packaging signal are removed and incorporated into a heterologous promoter to reduce recombination potential. The second-generation LVVs have the accessory genes (vif, vpr, vpu, and nef) removed to reduce the virulence of any potential replication-competent retrovirus. Additionally, the third-generation LVVs have the transactivator gene, tat, eliminated, which splits the vector into 3 plasmids to reduce further recombination potential, retaining only the 3 genes necessary for transgene expression (gag, pol, rev). Fourth-generation LVVs split the gag and pol onto separate plasmids to reduce even further recombination potential. This generation also added back some HIV genes to enhance transduction efficiency and transgene expression.

A detailed list of lentiviruses may be found in Coffin et al (1997) “Retroviruses” Cold Spring Harbor Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

The lentivirus family differs from retroviruses in that lentiviruses have the capability to infect both dividing and non-dividing cells (Lewis et al. (1992); Lewis and Emerman (1994)). In contrast, retroviruses, such as MLV, are unable to infect non-dividing or slowly dividing cells such as those that make up, for example, muscle, brain, lung and liver tissue.

Bioproduction of integrative but replication-defective lentiviral vectors is based on the separation of cis- and trans-acting sequences of the lentivirus. Transduction in non-dividing cells requires the presence of two cis-acting sequences in the lentiviral genome, the central polypurine tract (cPPT) and the central termination sequence (CTS). This leads to the formation of a triple-stranded DNA “flap”, which maximizes the efficiency of gene import into the nuclei of non-dividing cells, including dendritic cells (DCs) (Zennou et al., 2000, Cell, 101 (2) 173-85; Arhel et al., 2007, EMBO J, 26(12):3025-25 37). Furthermore, removal of the LTR U3 sequence has resulted in “self-inactivating” vectors which are entirely devoid of viral promoter and enhancer sequences and are safer.

Lentiviral particles, which contain lentiviral vectors, can be produced by transient transfection of HEK 293 cells (with or without the T antigen, adherent or grown in suspension) using a combination of DNA plasmids, for example: (i) a packaging plasmid expressing Gag, Pol, Rev, Tat, and optionally structural proteins or enzymes necessary for packaging of the transfer construct; (ii) a proviral transfer plasmid containing an expression cassette and HIV cis-acting factors necessary for packaging, reverse transcription, and integration; and (iii) a plasmid

encoding an envelope protein, such as the glycoprotein of vesicular stomatitis virus (VSV-G), a protein that allows the formation of mixed particles (pseudotypes) that can transduce a wide variety of cells, especially antigen-presenting cells (APCs), including dendritic cells (DCs) and macrophages. In the present technology, the plasmid encoding an envelope protein is omitted completely in certain embodiments, while in other embodiments it is included but its expression is maintained at a low level. Lentiviral particle vectors can also be produced continuously by stably inserting the packaging genes and proviral coding DNA into the cellular genome. A combination of integrated plasmids and transient transfection also can be used.

Lentiviral vectors devoid of envelope protein have also been used as gene delivery vehicles. Particularly, non-integrating lentiviral vectors lacking the envelope protein are also used as gene transfer vehicles. Examples of non-integrating lentiviral vectors are found in Coutant et al., PLOS ONE 7(11):e48644 (2102); Karwacz et al., J. Virol. 83(7):3094-3103 (2009); Negri et al., Molecular Therapy 15(9): 1716-1723 (2007); and Hu et al., Vaccine 28:6675-6683 (2010).

As the 5′LTR of the proviral sequence is devoid of U3, the expression of the transgene is driven by an internal promoter. Viral promoters, such as the CMV promoter, preferably are not used because of the presence of strong enhancers. Ubiquitous promoters can be used, such as promoters for the human genes encoding ubiquitin, PGK, b-actin, GAPDH, b-kinesin, and the like. Alternatively, promoters active in T-cells and APCs can be used, such as the promoter for human MHC class I genes. In all cases, the promoter preferably does not contain an enhancer. The promoter is preferably selected so as to achieve a therapeutic level of expression of the transgene in the target cells. In some embodiments, the promoter is specific for the target cells, i.e. , it enables a therapeutic expression level of the transgene in the target cells, and preferably enables a higher level of transgene expression in target cells than in non-target cells; In certain embodiments, the promoter allows little or no expression of the transgene in non-target cells.

Lentiviral vectors are used in clinical applications due to their ability to more efficiently transduce nonproliferating or slowly proliferating cells, such as CD34+ stem cells. Gene transfer using LVVs into CD34+ HSCs has been used to treat several genetic diseases, including β-thalassemia,100 X-linked adrenoleukodystrophy, metachromatic leukodystrophy, and Wiskott-Aldrich Syndrome. Recently, multiple clinical trials used third-generation, self-inactivating LVVs to introduce genes into hematopoietic stem cells to correct primary immunodeficiencies and hemoglobinopathies. LVVs have been used to alter T cells by introducing genes to generate immunity to fight cancer through the delivery of chimeric antigen receptors (CARs) or cloned T-cell receptors. Also, CART-cell therapies developed using LVVs have been successful in the clinical setting in patients with B-cell malignancies. 3. Adeno-Associated Virus Vectors

In other alternative embodiments adeno-associated virus vectors are potentially used to produce genetically modified T cells according to the invention. Adeno-associated virus vectors are nonpathogenic parvoviruses, consisting of a linear single-stranded DNA (ssDNA) genome of approximately 4.7 kilobases (kb), with two 145 nucleotide-long inverted terminal repeats (ITRs) at the termini. The virus does not encode a polymerase and therefore relies on cellular polymerases for genome replication.

The AAV genome consists of 3, open-reading frames (ORF) flanked by ITRs. The ITRs function as the viral origin of replication (rep) and the packaging signal (cap). The rep ORF encodes 4 nonstructural proteins. These structural proteins play a role in viral replication, transcriptional regulation, genomic integration, and virion assembly. The cap ORF encodes 3 structural proteins (VP1, VP2, and VP3) that assemble to form a 60-mer (repeat unit) viral capsid. Finally, an ORF that is present as an alternate reading frame within the cap gene produces the assembly-activating protein, a viral protein that localizes AAV capsid proteins to the nucleolus and functions in the capsid assembly process. In recombinant forms of AAV, the gene of interest is inserted between the ITRs in place of rep and cap, and the latter is provided in trans, along with helper viral genes, during vector production. The significant advantage is that the resulting vector can transduce both dividing and nondividing cells, with stable transgene expression in the absence of the helper virus in postmitotic tissue. There are 11 naturally occurring serotypes and more than 100 variants of AAV that differ in amino acid sequence, particularly within the hypervariable regions of the capsid proteins. As a result, these variants of AAV vary somewhat in their gene delivery properties, which is utilized successfully in several gene therapy applications.

AAV is one of the most common vectors used in gene therapy and reportedly provides for long-term and efficient transgene expression in various cell types in many tissues such as liver, muscle, retina, and the central nervous system (CNS).

### 4. Adenovirus Vectors

In another alternative embodiment adenoviral vectors are potentially used to produce genetically modified T cells according to the invention. Adenoviruses are a class of DNA viruses with a double-stranded 34 kb to 43 kb genome, which employs alternative splicing to encode genes in both the sense and antisense orientations. The AV genome is flanked by 2 ITRs and contains 8 transcription units.64 The early regions (E1A, E1B, E2, E3, and E4) are the first regions transcribed and encode proteins involved in activating transcription of other viral regions and altering the cellular environment to promote viral production. The late regions (L1-L5) are transcribed from an alternatively spliced transcript. Similar to recombinant AAV vectors, the AV genome remains in an extrachromosomal form following infection. Humans have 51 different serotypes of AV; 45% to 80% of the population harbors neutralizing antibodies against Ad5, the most prevalent, due to natural infections, which typically date back to infancy.

Standard recombinant adenoviral vectors have the E1 unit deleted, but the removal of E3 can create vectors capable of packaging up to 7.5 kb of foreign DNA. Vectors with both E1 and E4 units deleted result in lower immune responses. In helper-dependent, or gutless, adenoviral vectors, all adenoviral genes are deleted, and a helper virus provides adenoviral genes for production. Although these adenoviral genes have a carrying capacity of more than 30 kb, they are currently challenging to manufacture. Another concern with adenoviral vectors, in general, is that the particles themselves elicit cellular inflammatory responses as opposed to relatively inert AAV virions.

Compared with some other viral gene delivery systems, adenoviral vectors offer significant advantages. First, the AV is a very effective means of delivering genes in vivo as most human cells express the primary AV receptor and the secondary integrin receptors. Thus, human cells are easily infected with AV vectors and consequently yield high levels of the transgene expression. Second, the development of gutless adenoviral vectors allows circumvention of anti-adenoviral vector immunity. Previous believed inherited shortcomings of AV, such as immunity evoked against the AV capsid and low-level expression of AV genes, may now prove beneficial for the development of anticancer immunotherapies, where inducing immunity against cancer or directly killing the cancer cell is the goal.

### 5. Herpesvirus Vectors

In yet another alternative embodiment herpesvirus vectors are potentially used to produce genetically modified T cells according to the invention. Herpes simplex virus is an enveloped virus with a dsDNA genome more than 150 kb in length, which is divided into long and short unique segments (UL and US, respectively) and flanked by inverted repeated sequences (TRL/IRL and TRS/IRS). The HSV genome encodes approximately 90 genes, almost half of which are nonessential and can be eliminated in recombinant vectors.

Currently, gene delivery and gene therapy applications use 3 types of HSV vectors. First, amplicons are gene delivery systems packaged through transfection into producer cells of plasmids containing the HSV origin of DNA replication, HSV cleavage-packaging sequences, and transgene(s) of interest. The plasmids form head-to-tail linked concatemers that are packaged into the viral particles, which can, in general, accommodate large fragments of foreign DNA. In a second system, the deletion of genes necessary for the lytic cycle of HSV can create less toxic, replication-defective vectors that elicit a smaller immune response than wild-type HSV vectors. Deleting various combinations of the immediate, early genes that otherwise lead to the expression of the HSV infected cell proteins (ICPO, ICP4, ICP22, ICP27, and ICP47) creates several versions of replication-defective HSV vectors. Finally, replication-competent HSV vectors are attenuated versions of HSV that contain the genes required for replication in vitro but lack the genes necessary for replication in vivo. Replication-competent vectors serve as oncolytic therapies and vaccines, where the set of deleted genes must lead to the appropriate balance of replicative attenuation for safety and lytic activity for tumor toxicity or vaccine immunogenicity, respectively.

HSV-1 is a neurotropic virus that displays numerous necessary adaptations to the nervous system. With regard to the neurological application, the design of gene therapy vectors can exploit each adaptation rationally. HSV vectors containing latency-active promoters LAP1 and LAP2 have been engineered for long-term expression (up to 300 days) in the peripheral nervous system. HSV vectors have also found applications in the CNS. However, because wild-type HSV causes encephalitis, developing nontoxic replication-defective vectors with deletions in the IE genes (ICP4, ICP22, ICP27) is essential.

### B. Use of Gene Editing for Producing Modified T Cells According to the Invention

Genome editing, or genome engineering, or gene editing, is a type of genetic engineering in which DNA is inserted, deleted, modified or replaced in the genome of a living organism. Unlike early genetic engineering techniques that randomly inserts genetic material into a host genome, genome editing targets the insertions to site-specific locations. The basic mechanism involved in genetic manipulations through programmable nucleases is the recognition of target genomic loci and binding of effector DNA-binding domain (DBD), double-strand breaks (DSBs) in target DNA by the restriction endonucleases (Fokl and Cas), and the repair of DSBs through homology-directed recombination (HDR) or non-homologous end joining (NHEJ).

The key to genome editing is creating a DSB at a specific point within the genome. Commonly used restriction enzymes are effective at cutting DNA, but generally recognize and cut at multiple sites. To overcome this challenge and create site-specific DSB, three distinct classes of nucleases have been discovered and bioengineered to date. These are the Zinc finger nucleases (ZFNs), transcription-activator like effector nucleases (TALEN), meganucleases and the clustered regularly interspaced short palindromic repeats (CRISPR/Cas9) system. Different gene editing techniques and enzymes which potentially may be used to produce modified T cells according to the invention are briefly described below.

### 1. Meganucleases

Meganucleases, discovered in the late 1980s, are enzymes in the endonuclease family which are characterized by their capacity to recognize and cut large DNA sequences (from 14 to 40 base pairs). The most widespread and best known meganucleases are the proteins in the LAGLIDADG family, which owe their name to a conserved amino acid sequence.

Meganucleases, found commonly in microbial species, have the unique property of having very long recognition sequences (>14 bp) thus making them naturally very specific. However, there is virtually no chance of finding the exact meganuclease required to act on a chosen specific DNA sequence. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting amino acids of the meganuclease to design sequence specific meganucelases in a method named rationally designed meganuclease. Another approach involves using computer models to try to predict as accurately as possible the activity of the modified meganucleases and the specificity of the recognized nucleic sequence.

A large bank containing several tens of thousands of protein units has been created. These units can be combined to obtain chimeric meganucleases that recognize the target site, thereby providing research and development tools that meet a wide range of needs (fundamental research, health, agriculture, industry, energy, etc.) These include the industrial-scale production of two meganucleases able to cleave the human XPC gene; mutations in this gene result in Xeroderma pigmentosum, a severe monogenic disorder that predisposes the patients to skin cancer and burns whenever their skin is exposed to UV rays.

Meganucleases have the benefit of causing less toxicity in cells than methods such as Zinc finger nuclease (ZFN), likely because of more stringent DNA sequence recognition; however, the construction of sequence-specific enzymes for all possible sequences is costly and time-consuming, as one is not benefiting from combinatorial possibilities that methods such as ZFNs and TALEN-based fusions utilize. 2. Zinc finger nucleases

As opposed to meganucleases, the concept behind ZFNs and TALEN technology is based on a non-specific DNA cutting catalytic domain, which can then be linked to specific DNA sequence recognizing peptides such as zinc fingers and transcription activator-like effectors (TALEs). The first step to this was to find an endonuclease whose DNA recognition site and cleaving site were separate from each other, a situation that is not the most common among restriction enzymes.[30] Once this enzyme was found, its cleaving portion could be separated which would be very non-specific as it would have no recognition ability. This portion could then be linked to sequence recognizing peptides that could lead to very high specificity.

Zinc finger motifs occur in several transcription factors. The zinc ion, found in 8% of all human proteins, plays an important role in the organization of their three-dimensional structure. In transcription factors, it is most often located at the protein-DNA interaction sites, where it stabilizes the motif. The C-terminal part of each finger is responsible for the specific recognition of the DNA sequence.

The recognized sequences are short, made up of around 3 base pairs, but by combining 6 to 8 zinc fingers whose recognition sites have been characterized, it is possible to obtain specific proteins for sequences of around 20 base pairs. It is therefore possible to control the expression of a specific gene. It has been demonstrated that this strategy can be used to promote a process of angiogenesis in animals. It is also possible to fuse a protein constructed in this way with the catalytic domain of an endonuclease in order to induce a targeted DNA break, and therefore to use these proteins as genome engineering tools.

The method generally adopted for this involves associating two DNA binding proteins—each containing 3 to 6 specifically chosen zinc fingers—with the catalytic domain of the Fokl endonuclease which need to dimerize to cleave the double-strand DNA. The two proteins recognize two DNA sequences that are a few nucleotides apart. Linking the two zinc finger proteins to their respective sequences brings the two Fokl domains closer together. Fokl requires dimerization to have nuclease activity and this means the specificity increases dramatically as each nuclease partner would recognize a unique DNA sequence. To enhance this effect, Fokl nucleases have been engineered that can only function as heterodimers.

Several approaches are used to design specific zinc finger nucleases for the chosen sequences. The most widespread involves combining zinc-finger units with known specificities (modular assembly). Various selection techniques, using bacteria, yeast or mammal cells have been developed to identify the combinations that offer the best specificity and the best cell tolerance. Although the direct genome-wide characterization of zinc finger nuclease activity has not been reported, an assay that measures the total number of double-strand DNA breaks in cells found that only one to two such breaks occur above background in cells treated with zinc finger nucleases with a 24 bp composite recognition site and obligate heterodimer Fokl nuclease domains.

The heterodimer functioning nucleases would avoid the possibility of unwanted homodimer activity and thus increase specificity of the DSB. Although the nuclease portions of both ZFNs and TALEN constructs have similar properties, the difference between these engineered nucleases is in their DNA recognition peptide. ZFNs rely on Cys2-His2 zinc fingers and TALEN constructs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. Each finger of the Zinc finger domain is completely independent and the binding capacity of one finger is impacted by its neighbor. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

Zinc finger nucleases are research and development tools that have already been used to modify a range of genomes, in particular by the laboratories in the Zinc Finger Consortium. The US company Sangamo BioSciences uses zinc finger nucleases to carry out research into the genetic engineering of stem cells and the modification of immune cells for therapeutic purposes. Modified T lymphocytes are currently undergoing phase I clinical trials to treat a type of brain tumor (glioblastoma) and in the fight against AIDS.

### 3. Talen

Transcription activator-like effector nucleases (TALENs) are specific DNA-binding proteins that feature an array of 33 or 34-amino acid repeats. TALENs are artificial restriction enzymes designed by fusing the DNA cutting domain of a nuclease to TALE domains, which can be tailored to specifically recognize a unique DNA sequence. These fusion proteins serve as readily targetable “DNA scissors” for gene editing applications that enable to perform targeted genome modifications such as sequence insertion, deletion, repair and replacement in living cells.[36] The DNA binding domains, which can be designed to bind any desired DNA sequence, comes from TAL effectors, DNA-binding proteins excreted by plant pathogenic Xanthomanos app. TAL effectors consists of repeated domains, each of which contains a highly conserved sequence of 34 amino acids, and recognize a single DNA nucleotide within the target site. The nuclease can create double strand breaks at the target site that can be repaired by error-prone non-homologous end-joining (NHEJ), resulting in gene disruptions through the introduction of small insertions or deletions. Each repeat is conserved, with the exception of the so-called repeat variable di-residues (RVDs) at amino acid positions 12 and 13. The RVDs determine the DNA sequence to which the TALE will bind. This simple one-to-one correspondence between the TALE repeats and the corresponding DNA sequence makes the process of assembling repeat arrays to recognize novel DNA sequences straightforward. These TALEs can be fused to the catalytic domain from a DNA nuclease, Fokl, to generate a transcription activator-like effector nuclease (TALEN). The resultant TALEN constructs combine specificity and activity, effectively generating engineered sequence-specific nucleases that bind and cleave DNA sequences only at pre-selected sites. The TALEN target recognition system is based on an easy-to-predict code. TAL nucleases are specific to their target due in part to the length of their 30+ base pairs binding site. TALEN can be performed within a 6 base pairs range of any single nucleotide in the entire genome.

TALEN constructs are used in a similar way to designed zinc finger nucleases, and have three advantages in targeted mutagenesis: (1) DNA binding specificity is higher, (2) off-target effects are lower, and (3) construction of DNA-binding domains is easier.

### 4. CRISPR

CRISPRs (Clustered Regularly Interspaced Short Palindromic Repeats) are genetic elements that bacteria use as a kind of acquired immunity to protect against viruses. They consist of short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position.

CRISPR gene editing is a genetic engineering technique in molecular biology by which the genomes of living organisms may be modified. It is based on a simplified version of the bacterial CRISPR-Cas9 antiviral defense system. By delivering the Cas9 nuclease complexed with a synthetic guide RNA (gRNA) into a cell, the cell's genome can be cut at a desired location, allowing existinggenes to be removed and/or new ones added in vivo.

Working like genetic scissors, the Cas9 nuclease opens both strands of the targeted sequence of DNA to introduce the modification by one of two methods. Knock-in mutations, facilitated via homology directed repair (HDR), is the traditional pathway of targeted genomic editing approaches. This allows for the introduction of targeted DNA damage and repair. HDR employs the use of similar DNA sequences to drive the repair of the break via the incorporation of exogenous DNA to function as the repair template. This method relies on the periodic and isolated occurrence of DNA damage at the target site in order for the repair to commence. Knock-out mutations caused by CRISPR-Cas9 result in the repair of the double-stranded break by means of non-homologous end joining (NHEJ).

### 5. Editing by Nucleobase Modification (BASE Editing)

Another method of efficiently editing nucleic acids employs nucleobase modifying enzymes directed by nucleic acid guide sequences was first described in the 1990s and has seen resurgence more recently. This method has the advantage that it does not require breaking the genomic DNA strands, and thus avoids the random insertion and deletions associated with DNA strand breakage. It is only appropriate for precise editing requiring single nucleotide changes and has found to be highly efficient for this type of editing.

### 6. ARCUT

ARCUT stands for artificial restriction DNA cutter, it is a technique developed by Komiyama. This method uses pseudo-complementary peptide nucleic acid (pcPNA), for identifying cleavage site within the chromosome. Once pcPNA specifies the site, excision is carried out by cerium (CE) and EDTA (chemical mixture), which performs the splicing function.

### Exemplary Embodiments

In exemplary embodiments, the invention comprises a method of preparing an engineered T cell comprising the steps of: inserting a DNA sequence which encodes either at least one copy of a PDP1 gene and/or at least one copy of a PDK1 into a parental retroviral vector; optionally inserting a transgene encoding another moiety into the retroviral vector such as a polynucleotide encoding a CAR or a desired polypeptide such as a hormone, cytokine, or other immunomodulatory polypeptide; transfecting the resultant modified retroviral genome into suitable mammalian cells, culturing said cells to produce virus particles comprising said PDP1 or PDK1 gene, harvesting and/or purifying said retroviral virus particles from the culture medium, contacting said virus particles with a T cell to form or produce a virally-transduced T cell; growing the viral-transduced T cells, harvesting and/or purifying the virally-transduced cells, and optionally, freezing said cells so as to preserve their viability.

In preferred embodiments, the method of the invention is effected using human T cells, preferably primary human T cells. In some preferred embodiments, the method of the invention is effected using CD4+ T cells. In most preferred embodiments, the method of the invention is effected using CD8+ T cells.

In some embodiments, the retroviral vector comprises additional nucleic acid sequences which facilitate the identification or purification of the cells comprising the transduced PDP1 or PDK1 gene. In an exemplary embodiment, the additional nucleic acid sequences may comprise coding sequences for CD19.

In some embodiments, the retroviral vector and engineered T cells of the invention comprise mammalian PDP1 or PDK1 genes and other coding sequences, e.g., those encoding a desired receptor or antigen binding domain, a CAR, a desired therapeutic polypeptide or a desired immunomodulatory polypeptide such as a cytokine, hormone, antitumor agent. In some embodiments the PDP1 or PDK1 genes and other coding sequences are derived from murine nucleic acid sequences. In preferred embodiments, the PDP1 or PDK1 genes and other coding sequences are derived from human nucleic acid sequences.

In some embodiments, the engineered T cells of the invention are derived from mammalian T cells. In some embodiments the engineered T cells of the invention are derived from murine T cells. In preferred embodiments, the engineered T cells of the invention are derived from human T cells, preferably primary CD4+ and/or CD8+ T cells of a subject who is in need of ACT or an allogeneic human T cell donor.

Retroviral vector or retroviral vector particle of the present invention may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV)5 Moloney murine sarcoma virus (Mo-MSV), Abelson murine leukemia virus (A-MLV), Avian myelocytomatosis virus-29 (MC29), and Avian erythroblastosis virus (AEV)5 Foamy virus (FMV). A detailed list of retroviruses may be found in Coffin et al. (1997) “Retroviruses”, Cold Spring Harbor Laboratory Press Eds: J M Coffin, SM Hughes, HE Varmus pp 758-763.

Retroviruses may be broadly divided into two categories: namely, “simple” and “complex”. Retroviruses may even be further divided into seven groups. Five of these groups represent retroviruses with oncogenic potential. A review of these retroviruses is presented in Coffin et al. (1997) (ibid).

The basic structure of retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and envgenes encoding the packaging components—these are polypeptides required for the assembly of viral particles. Integrase is encoded by the 3′ end of the pol gene, which also codes for two other viral enzymes, the protease and the reverse transcriptase. These three enzymes are initially synthesized as part of a larger polyprotein that is subsequently cleaved by the protease into the individual proteins.

Lentiviruses have additional features, such as the rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

### Reverse Transcription

Once the viral core enters the cytoplasm of the target cell, reverse transcription converts viral genomic RNA into double stranded DNA. Reverse transcriptase initiates minus-strand DNA synthesis by elongating a partially unwound primer tRNA that is hybridized to the primer binding site (PBS) in genomic RNA.

In HIV-I, tRNALYS3 serves as the replication primer. Synthesis continues to the 5′ end of the genome, generating minus-strand DNA [(-)ssDNA]. As reverse transcriptase reaches the end of the template, its RNase H activity degrades the RNA strand of the RNA/DNA duplex. This allows the first strand transfer to proceed whereby (-)ssDNA is transferred to the 3′ end of genome, guided by the repeat (R) sequences of the LTRs present on both ends of the RNA. Minus-strand DNA synthesis then resumes and is completed by reverse transcriptase, again accompanied by RNase H-mediated degradation of the template strand. Template digestion is incomplete and results in the generation of RNase H-resistant oligoribonucleootides rich in purines, called the polypurine tract (PPT). Plus-strand DNA synthesis is primarily at the PPT and then proceeds by copying minus-strand DNA to its 5′ end. RNase H removal of the primer tRNA facilitates the second strand transfer, in which complementary PBS segments in the plus-strand DNA and in the minus-strand DNA anneal. The plus and minus strand syntheses are then completed, with each strand serving as a template for the other. On completion of the reverse transcription, the viral DNA is translocated into the nucleus where the linear copy of the viral genome, called a preintegration complex (PIC), is inserted into chromosomal DNA with the aid of the virion integrase to form a stable provirus. The number of possible sites of integration into the host cellular genome is very large and very widely distributed.

The term ‘incapable of undergoing reverse transcription’ used herein means the viral genome is not able to undergo reverse transcription via the conventional retroviral or lenti viral reverse transcription mechanism, such as that described above.

### Integration

Integrase first acts within the preintegration complex by mediating an endonucleolytic cleavage at the 3′ end of each strand of viral DNA immediately beyond a conserved subterminal CA dinucleotide. This step, called 3′-processing, occurs in the cytoplasm and leaves a terminal hydroxyl group at the 3′ end of each strand of viral DNA. After the nucleoprotein complex migrates to the nucleus, integrase mediates a concerted nucleophilic attack involving the viral 3′ hydroxyl residues and phosphate residues on either side of the major groove in the target DNA, a step termed strand transfer. The two viral ends attack the target DNA in a coordinated, 5′-staggered fashion, the extent of the stagger determining the length of the virus-specific direct repeat of host DNA that flanks the integrated provirus.

Attachment (att) sites, virus-specific sequences located at each end of viral DNA, and integrase, are known to be essential for integration (Gaur et al. (1988) J Virol. 72(6): 4678-4685). Coupled with amino acid sequence alignment, the in vitro activity data for wild-type and mutant integrase proteins have led to a working model of integrase with three domains: the amino-terminal or HHCC domain, the core or catalytic domain, and the carboxy-terminal or DNA binding domain (Gaur et al. (1988) J Virol. 72(6):4678-4685).

The terms ‘incapable of undergoing integration’, or ‘integration defective’ used herein mean the viral genome is not able to integrate into the target cell genome via the conventional retroviral or lentiviral integration mechanism, such as that described above.

Important to the catalytic activity of the integrase is the highly conserved DDE motif found in all retroviral integrase proteins and numerous transposable elements. The DDE motif refers to three absolutely conserved acidic amino acids (two aspartic acids and one glutamic acid) in the order indicated, with a conserved spacing of generally 35 amino acids between the second and third residues (Engelman and Craigie (1992) J Virol. 66:6361-6369; Johnson et al. (1986) Proc. Natl. Acad. Set USA 83:7648-7652; Khan et al. (1991) Nucleic Acids Res. 19:851-860).

In a defective retroviral or lentiviral vector genome gag, pol and env may be absent or not functional. The R regions at both ends of the RNA are repeated sequences. U5 and U3 represent unique sequences at the 5′ and 3′ ends of the RNA genome respectively.

In a typical viral vector of the present invention, at least part of one or more protein coding regions essential for replication may be removed from or disabled in the virus.

This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target non-dividing host cell and/or integrating its genome into a host genome.

A detailed list of lentiviruses may be found in Coffin et al (1997) “Retroviruses” Cold Spring Harbor Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

The lentivirus family differs from retroviruses in that lentiviruses have the capability to infect both dividing and non-dividing cells

### Further Genetic Modification of Engineered T Cells According to the Invention

In some preferred embodiments the engineered T cells of the invention are further engineered to comprise and express a chimeric antigen receptor (CAR). The CAR may comprise: (a) an antigen-binding (AB) domain that binds to a desired antigen, e.g., a tumor antigen, (b) a transmembrane (TM) domain, and (c) an intracellular signaling (ICS) domain. Optionally, such a CAR may further comprise (d) a hinge that joins said AB domain and said TM domain, and (e) one or more costimulatory (CS) domains. The CAR may be introduced into the T cells using the same retroviral genetic construct as the PDP1 or PDK1 gene or by use of a different genetic construct, which optionally may comprise a retroviral construct.

In some embodiments, the AB domain of the CAR may be any Ab or antigen-binding Ab fragment. In some particular embodiments, the AB domain may be an scFv.

In some embodiments, the ICS domain of the CAR if present may be derived from a cytoplasmic signaling sequence, or a functional fragment thereof, of for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, or DAP12.

In certain embodiments, the ICS domain of the CAR if present may be derived from a cytoplasmic signaling sequence of CD3z, or a functional fragment thereof.

In some embodiments, the hinge of the CAR may be derived from CD28.

In some embodiments, at least one of the one or more CS domains of the CAR if present may be derived from a cytoplasmic signaling sequence, or functional fragment thereof, of for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand.

In certain embodiments, the CS domain of the CAR may be derived from a cytoplasmic signaling sequence of CD28, 4-1BB, or DAP10, or functional fragment thereof.

In some embodiments, the CAR may further comprise a cytotoxic drug conjugated to the AB domain.

In some embodiments, an isolated polynucleotide or combination of isolated polynucleotides encoding the CAR may encode an Ab or antigen-binding Ab fragment that is for example, but not limited to, a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, or a minibody.

In some instances, the encoded Fc region may or may be derived from the Fc region of a human IgM, a human IgD, a human IgG, a human IgE, or a human IgA, optionally of a human IgG1, a human IgG2, a human IgG3, or a human IgG4.

In some instances, the encoded human or human-like Fc region may bind to an Fc receptor (FcR). The FcR may be for example, but not limited to, Fc gamma receptor (FcgR), FcgRI, FcgRIIA, FcgRIIB1, FcgRIIB2, FcgRIIIA, FcgRlIIB, Fc epsilon receptor (FceR), FceRI, FceRII, Fc alpha receptor (FcaR), FcaRI, Fc alpha/mu receptor (Fca/mR), or neonatal Fc receptor (FcRn).

In some embodiments, the encoded CAR may comprise: (a) an AB domain that binds to a desired antigen; (b) a transmembrane (TM) domain; (c) an intracellular signaling (ICS) domain; (d) optionally a hinge that joins said AB domain and said TM domain; and (e) optionally one or more costimulatory (CS) domains.

In some embodiments, the polynucleotide may encode a CAR whose ICS domain is derived from a cytoplasmic signaling sequence, or a functional fragment thereof, of, for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some embodiments, the isolated polynucleotide may encode a CAR whose at least one of the one or more CS domains is derived from a cytoplasmic signaling sequence, or functional fragment thereof, of, for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand. In certain embodiments, the CS domain may be derived from a cytoplasmic signaling sequence of CD28, 4-1BB, or DAP10, or functional fragment thereof.

In some preferred embodiments the engineered T cells of the invention are further engineered to comprise and express a gene encoding one or more other therapeutic moieties such as a hormone, cytokine, cytokine antagonist, therapeutic antibody, immunostimulatory antibody or fusion protein, immunoinhibitory antibody or fusion protein, anti-inflammatory agent, checkpoint inhibitor or checkpoint inhibitor agonist or antagonist, e.g., a polypeptide which promotes or reduces the expression or activity of a stimulatory immune checkpoint molecule such as CD27, CD28, CD40, CD122, CD137, OX40, GITR or ICOS and/or a polypeptide which promotes or reduces the expression or activity of an inhibitory immune checkpoint molecule such as A2AR, A2BR, B7-H3, B7-H4, BTLA, CTLA-4, IDO, KIR, LAG3, NOX2, PD-1, PD-L1, VISTA or SIGLEC7).

In some preferred embodiments the engineered T cells of the invention are further engineered to comprise and express a gene encoding IL-2, IL-4, IL-5, IL-7, IL-10, IL-12p40, IL-12p70, IL-15, and interferon (IFN) gamma. In some preferred embodiments the engineered Tcells of the invention are further engineered to comprise and express a gene encoding a pro-inflammatory cytokine, e.g., IL-1a, IL-1b, IL-6, IL-13, IL-17a, tumor necrosis factor (TNF)-alpha TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF).

In some preferred embodiments the engineered T cells of the invention are further engineered to comprise and express a gene encoding an effector such as granzyme A, granzyme B, soluble Fas ligand (sFasL), or perforin.

In some preferred embodiments the engineered T cells of the invention are further engineered to comprise and express a gene encoding cytokines such as chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), granulocyte macrophage colony-stimulating factor (GM-CSF), leukemia inhibitory factor (LIF), oncostatin M (OSM), CD154, lymphotoxin (LT) beta, 4-1BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

In some preferred embodiments the engineered T cells of the invention are further engineered such that they comprise impaired or no expression of an endogenous polypeptide, e.g., the endogenous T cell receptor (TCR) and/or endogenous MHC receptor, thereby permitting the T cells to be used in allogeneic subjects. This can be effected using known methods such as use of shRNAs, siRNAs, CRSPR, and other gene editing methods.

II. T Cells Having Upregulated Mitochondrial and/or Glycolytic Metabolism According to the Invention

The T cells of the invention are generally mammalian T cells, and typically are human Tcells, e.g., those derived from human subjects and modified, for example, to enhance mitochondrial and/or glycolytic metabolism via expression of pyruvate dehydrogenase phosphatase (PDP) or pyruvate dehydrogenase kinase (PDK). In some embodiments, the cells are derived from the blood, bone marrow, lymph, or lymphoid organs, typically T cells, particularly CD8+ T cells. Other exemplary cells include stem cells, such as multipotent and pluripotent stem cells, including induced pluripotent stem cells (iPSCs) and progeny cells derived from them. The cells typically are primary cells, such as those isolated directly from a subject and/or isolated from a subject and frozen. In some embodiments, the cells include one or more subsets of Tcells or other cell types, such as whole T cell populations, CD8+ cells, CD4+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some embodiments, the methods include isolating cells from the subject, preparing, processing, culturing, and/or engineering them, and re-introducing them into the same subject, before or after cryopreservation of the cells.

Among the sub-types and subpopulations of T cells and/or of CD8+ and/or of CD4+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helperTcells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In exemplary embodiments, the engineered cells of the invention comprise human T cells. In some embodiments, cells of the invention comprise human CD4+ T cells. In preferred embodiments, the engineered cells of the invention comprise human CD8+ T cells.

In some embodiments, the engineered cells of the invention comprising additional nucleic acid sequences which facilitate the identification or purification of the engineered cells. In an embodiment, the additional nucleic acid sequence comprises coding sequences for CD19.

In some embodiments, the engineered T cells of the invention comprise mammalian PDP1 or PDK1 genes and other coding sequences. In embodiments the PDP1 or PDK1 genes and other coding sequences are derived from murine nucleic acid sequences. In preferred embodiments, the PDP1 or PDK1 genes and other coding sequences are derived from human nucleic acid sequences.

In embodiments, the engineered T cells of the invention are derived from mammalian T cells. In embodiments the engineered T cells of the invention are derived from murine T cells. In preferred embodiments, the engineered T cells of the invention are derived from human T cells.

In embodiments, the engineered cells of the invention are optionally further modified to express an exogenous TCR or a CAR. In embodiments, the engineered cells of the invention are optionally modified to express a cytokine, a chemokine, a cytokine receptor, a chemokine receptor, an immune checkpoint inhibitor or immune checkpoint antagonist protein.

Also provided in the invention are pharmaceutically acceptable compositions and formulations suitable for the preservation and use of therapeutic cells. Such compositions comprise e.g., sodium chloride, dextran 40, Plasma-Lyte A (trademark of Baxter International; per 100 mL, 526 mg of Sodium Chloride, USP (NaCl); 502 mg of Sodium Gluconate; 368 mg of Sodium Acetate Trihydrate, USP; 37 mg of Potassium Chloride, USP (KCl); and 30 mg of Magnesium Chloride, USP (MgCl2·6H2O), pH 7.4), buffers, human serum albumin, dextrose, and dimethyl sulfoxide. Compositions suitable for storing and administering cellular therapies are known in the art (see van der Walle et al. (2021) Pharmaceutics 13(8): 1317).

### III. Methods of Use of Inventive Engineered T Cells Having Upregulated Mitochondrial And/or Glycolytic Metabolism

Engineered T cells according to the invention and compositions containing are useful in treating any condition where adoptive T cell therapy is potentially useful. In particular, engineered T cells according to the invention and compositions containing are useful in cancer treatment, prevention and treatment of cancer reoccurrence, treatment of metastasis, treatment of infection, treatment autoimmunity, treatment of inflammation, treatment of neurodegenerative diseases and the like.

These methods will comprise the administration of a therapeutically effective amount or prophylactically effective amount of engineered T cells according to the invention.

In some embodiments, the method of treatment of the invention comprises administering engineered T cells which stably and/or heritably express one or more copies of a PDP1 and/or PDK1 gene(s) and which are optionally further modified to express an exogenous TCR or a CAR. In some embodiments, the method of treatment of the invention comprises administering engineered T cells which stably and/or heritably express one or more copies of a PDP1 and/or PDK1 gene(s) and which are optionally further modified to impair or eliminate the expression of function of the endogenous TCR and/or an HLA receptor, e.g., HLA Class I, or HLA Class II receptor thereby enabling the resultant T Cells to be used for adoptive T-cell Therapy in allogeneic subjects.

In some embodiments, the engineered cells of the invention are optionally modified to express another moiety such as a cytokine, a chemokine, a cytokine receptor, a chemokine receptor, an immune checkpoint inhibitor or immune checkpoint antagonist protein. In some embodiments, the expression of a cytokine, a chemokine, a cytokine receptor, a chemokine receptor, an immune checkpoint inhibitor or immune checkpoint antagonist protein enhances the capacity of the cells of the invention to home in on disease-causing cells or microbes, to attack disease-causing cells or microbes, and/or to reduce or ameliorate symptoms of disease.

In some embodiments, the disease to be treated is a bacterial disease. Examples of infectious bacteria include, e.g., Helicobacter pylori, Borrelia burgdorferi, Legionella pneumophilia, Mycobacteria sp (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sp.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, Actinomyces israeli, and/or Chlamydia trachomatis.

In some embodiments, the disease to be treated is a viral disease. Examples of viruses causing such disease are, e.g., Retroviridae (for example, HIV); Picornaviridae (for example, polio viruses, hepatitis A virus; enteroviruses, human coxsackie viruses, rhinoviruses, echoviruses); Caliciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bunyaviridae (for example, Hantaan viruses, bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Epstein-Barr virus, Poxviridae (variola viruses, vaccinia viruses, pox viruses); and Asfarviridae (such as African swine fever virus); and Norwalk and related viruses.

In embodiments, the disease to be treated is a fungal disease. Examples of such diseases and fungi causing such disease are, e.g., aspergillosis; thrush; cryptococcosis (caused by Cryptococcus); and histoplasmosis, Cryptococcus neoformans, Histoplasma capsulatum, Coccidioides immitis, Blastomyces dermatitidis, and/or Candida albicans.

Other infectious organisms causing diseases which are contemplated to be treated by the methods of the invention include Plasmodium falciparum and Toxoplasma gondii.

In embodiments, the disease to be treated is a cancer. In some embodiments, the treated cancer may comprise any disease in which abnormal cells divide without control and which can invade nearby tissues or spread to other parts of the body through the blood and lymph systems. Cancer may include carcinomas (cancers that begin in the skin or in tissues that line or cover internal organs), sarcomas (cancers that begin in bone, cartilage, fat, muscle, blood vessels, or other connective or supportive tissue), leukemias (cancers that start in blood-forming tissue, such as the bone marrow, and causes large numbers of abnormal blood cells to be produced and enter the blood), lymphomas and multiple myelomas (cancers that begin in the cells of the immune system), and central nervous system cancers (cancers that begin in the tissues of the brain and spinal cord). Cancer may also refer to any malignancy. Types of cancer include but are not limited to adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

In some embodiments, the treated inflammatory condition may comprise Acne vulgaris, Allergy, Asthma, Ankylosing spondylitis, Antiphospholipid antibody syndrome, Atherosclerosis, Atopic dermatitis, Autoimmune diseases, Autoimmune encephalitis, Autoinflammatory diseases, Celiac disease, Chediak-Higashi syndrome, Chronic granulomatous disease, Chronic prostatitis, Chronic recurrent multifocal osteomyelitis, Colitis, Dermatomyositis, Diverticulitis, Endometriosis, Familial Mediterranean Fever, Fatty liver disease, Glomerulonephritis, Gout, Henoch-Schonlein purpura, Hidradenitis suppurativa, Hypersensitivities, Inclusion body myositis, Inflammatory bowel diseases, Interstitial cystitis, Juvenile dermatomyositis, Juvenile idiopathic arthritis, Juvenile lupus, Juvenile vasculitis, Kawasaki disease, Lichen planus, Mast Cell Activation Syndrome, Mastocytosis, Mixed connective tissue disease, Myositis, Osteoarthritis, Otitis, Pelvic inflammatory disease, Peripheral ulcerative keratitis, Pneumonia, Polymyositis, Psoriasis, Psoriatic arthritis, Reactive arthritis, Reperfusion injury, Rheumatic fever, Rheumatoid arthritis, Rhinitis, Sarcoidosis, Scleroderma, Sjogren's syndrome, Spondyloarthritis, Systemic juvenile idiopathic arthritis, Systemic lupus erythematosus, Systemic sclerosis, Transplant rejection, Type 1 diabetes mellitus, Type 2 diabetes mellitus, Undifferentiated connective tissue disease, Uveitis, and Vasculitis.

In some embodiments, the treated inflammatory condition or disease may be dermatologic inflammation. In exemplary embodiments, the dermatologic inflammation may be selected from eczema and psoriasis.

In some embodiments, the treated inflammatory condition or disease may be at least one autoimmune disease selected from Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type 11, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the composition may be administered by one or more of injection, optionally intravenous (IV) or subcutaneous (SC) administration, intratumoral administration, intrapleural administration, intracranial administration, nebulization, and/or eye drops. In a preferred embodiment, the composition is administered as an intravenous infusion.

The therapeutically effective amount may comprise one or more doses of the composition. In some embodiments, each dose may range from 0.1 mL/10 kg body weight to 10 mL/10 kg body weight. In some preferred embodiments, the dose may be 1 mL/10 kg body weight. Dosages may be modified or optimized based on criteria selected from a list comprising patient biometrics, specific combination of indications, and preferred route of administration. In some embodiments, each dose may range from 0.01-5 mL for local applications, preferably 1 mL administered SC or directly into locations such as tendons, ligaments, and joints. Each dose may range from 0.01-2 mL for topical eye indications, preferably 0.1 mL administered to each eye as eye drops. Each dose may range from 5-100 mL for systemic use, preferably 8 mL administered by IV infusion. Each dose may range from 0.5-5 mL for nebulization indications, preferably 3 mL administered as a nebulized mist.

In some embodiments, the subject may be selected from a human, non-human primate, pig, sheep, horse, cow, dog, cat, rat, and mouse. In preferred embodiments, the subject may be human.

In some embodiments, the subject has been treated with one or more additional cancer therapies prior to the administration of the modified cells. In some aspects, the subject may be or may have become refractory or non-responsive to the other treatment. In some embodiments, the subject may not have become refractory or non-responsive but the administration of the modified cells is carried out to complement the other treatment and/or enhance the subject's response to the other treatment. In some embodiments the modified cells are administered prior to or simultaneously with the other treatment. It is contemplated by this disclosure that the other treatment comprising one or more additional cancer therapies may include immunotherapy, chemotherapy, targeted therapy, stem cell transplant, radiation, surgery, and/or hormone therapy. In some embodiments, the immunotherapy may include immune checkpoint inhibitors (e.g., negative checkpoint blockade), monoclonal antibodies, cancer vaccines, immune system modulators, and/or adoptive cell therapies such as CAR-T cell therapy, exogenous TCR T cell therapy, and TIL therapy.

In some embodiments, the method of treatment or prevention may further comprise the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing.

In some embodiments, the cells are administered as part of a combination treatment, such as simultaneously with or sequentially with, in any order, another therapeutic intervention, such as an antibody or engineered cell or receptor or other agent, such as a cytotoxic, immunostimulatory, or therapeutic agent. Thus, the cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, IL-15, or other cytokine, for example, to enhance persistence. In some embodiments, the methods comprise administration of a chemotherapeutic agent, e.g., a conditioning chemotherapeutic agent, for example, to reduce tumor burden prior to the dose administrations, or an oncolytic virus.

In some embodiments, the subject may be subjected to lymphodepletion procedures prior to administration of the modified cells. In some embodiments, the subject may receive a nonmyeloablative lymphodepletion regimen or may undergo lymphodepletion with hematopoietic stem cell transplantation prior to administration of the modified cells. Methods to induce lymphopenia include treatment with low-dose total body irradiation (TBI) that produces mild, reversible myelosuppression (hence nonmyeloablative) and/or treatment with chemotherapeutic drugs such as cyclophosphamide (Cy) alone or in combination with fludarabine. Procedures for lymphodepletion are known in the art. See, e.g., Wrzesinski et al. (2007) J. Clin. Invest., 117(2):492-501.

In some embodiments, the methods described herein further comprise administering a chemotherapeutic agent to the subject. Non-limiting examples of chemotherapeutic agents can include alkylating agents such as thiotepa and cyclosphosphamide; alkyl sulfonates such as busulfan, improsulfan and piposulfan; aziridines such as benzodopa, carboquone, meturedopa, and uredopa; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin) dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., ABRAXANE (albumin-engineered nanoparticle formulation of paclitaxel), doxetaxel (Rhone-Poulenc Rorer, Antony, France); chloranbucil; gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-alpha, Raf, H-Ras, EGFR (e.g., erlotinib (Tarceva@)) and VEGF-A that reduce cell proliferation and pharmaceutically acceptable salts, acids or derivatives of any of the above. In addition, the methods of treatment can further include the use of radiation therapy.

In some embodiments the subject may receive a single dose of the modified cells. In some embodiments, the subject may receive multiple doses of the modified cells. In some embodiments, the cancer comprises a tumor and the subject has a large tumor burden prior to the administration of the first dose, such as a large solid tumor or a large number or bulk of tumor cells. In some aspects, the subject has a high number of metastases and/or widespread localization of metastases. In some aspects, the tumor burden in the subject is low and the subject has few metastases. In some embodiments, the size or timing of the doses is determined by the initial disease burden in the subject. For example, whereas in some aspects the subject may be administered a relatively low number of cells in a first dose, in the context of a higher disease burden, the dose may be higher and/or the subject may receive one or more additional doses.

In some embodiments, the persistence and in vivo expansion of the cells within the patient may be monitored to determine dosing and/or the effectiveness of the treatment. Methods of detecting and monitoring CAR-T cells in vivo by such methods as quantitative polymerase chain reaction and flow cytometry, are known in the art. See, e.g., Hu, Y., & Huang, J. (2020), Frontiers in Immunology, 11, 1770, Peinelt, A., et al. (2022) Frontiers in Immunology, 13, 8307733.

In some embodiments, for example, where the subject is a human, the dose includes fewer than about 1×10⁸ total engineered cells, e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose contains fewer than about 1×10⁸ total modified cells, engineered cells, T cells, or peripheral blood mononuclear cells (PBMCs) cells per m² of the subject, e.g., in the range of about 1×10⁶ to 1×10⁸ such cells per m² of the subject, such as 2×10⁶, 5×10⁶, 1×10⁷, 5×10⁷, or 1×10⁸ such cells per m² of the subject, or the range between any two of the foregoing values. In certain embodiments, the number of engineered cells in the first or subsequent dose is greater than about 1×10⁶ such cells per kilogram body weight of the subject, e.g., 2×10⁶, 3×10⁶, 5×10⁶, 1×10⁷, 5×107, 1×10⁸, 1×10⁹, or 1×10¹⁰ such cells per kilogram of body weight and/or, 1×10⁸, or 1×10⁹, 1×10¹⁰ such cells per m² of the subject or total, or the range between any two of the foregoing values.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; and in Rosenberg (2011) Nat Rev Clin Oncol. 8(10):577-85; Themeli et al. (2013) Nat Biotechnol. 31(10):928-933; Tsukahara et al. (2013) Biochem Biophys Res Commun 438(1): 84-9; Davila et al. (2013) PLoS ONE 8(4):e61338; and Wennhold et al., Transfus Med Hemother 2019; 46:36-46.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, orfrom a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical or similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, intrapleural injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, hepatic intravascular, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of cancer, the appropriate dosage may depend on the type of cancer to be treated, the type of modified cells, the type of recombinant receptors if present, the severity and course of the cancer, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In certain embodiments, the modified cells may be further modified in any number of ways, such that their therapeutic or prophylactic efficacy is increased. For example, the modified cells may express an endogenous cell surface receptor or may be engineered to express a cell surface receptor, such as an exogenous TCR or CAR, which can then be conjugated either directly or indirectly through a linker to a targeting moiety. The practice of conjugating compounds to targeting moieties is known in the art. See, for instance, Wadwa et al., J. Drug Targeting 3: 111 (1995), and U.S. Pat. No. 5,087,616.

Engineered T cells, compositions containing and/or methods for making and using disclosed and claimed herein can be made and executed without undue experimentation in light of the present disclosure. While the compositions and methods have been described in terms of preferred embodiments, it will be apparent to those of skill in the art that variations and substitutions may be applied to the compositions and/or methods and in the steps or in the sequence of steps of the methods described herein without departing from the concept, spirit and scope of the disclosure.

### V. EXAMPLES

The following examples are provided for illustrative purposes only and are non-limiting. Unless stated otherwise the following Materials and Methods were used in the Examples which follow.

### Materials and Methods

Mice, virus and bacteria. OT-1 mice were originally purchased from Jackson Laboratory (003831). CD45.1 mice were purchased from Jackson Laboratory (002014). MHV-68-Ova virus was kindly provided by Dr. Phillip Stevenson (University of Queensland, Australia). LM-actA-Ova was kindly provided by Dr. John Harty (University of Iowa).

Reagents: EasySep Mouse naïve CD8 T cell isolation kits (Stemcell Technologies cat:19858A); Mojosort mouse anti-APC nanobeads (Biolegend Cat:480072); ATP detection assay kit-luminescence (Cayman Chemical cat:700410); DAPI (Thermo Fisher cat:D1306); Seahorse XF Cell Mito Stress Test Kit (Seahorse Agilent cat:103015-100); 2-DG (Cayman Chemical cat:14325); SIINFEKL peptide (New England peptide Lot:V1355-37/40); recombinant human IL-2 (TECIN cat:Ro23-6019); recombinant murine IL-15 (PeproTech cat:210-15); poly-D-lysine (Millipore Sigma cat:P6407); Glutaraldehyde (Electron Microscopy Science cat:16000); NaBH4 (Alfa Aesar stock #:35788); Triton X-100 (PerkinElmer cat:N9300260).

### Example 1: Generation of Engineered T Cells (retroviral)

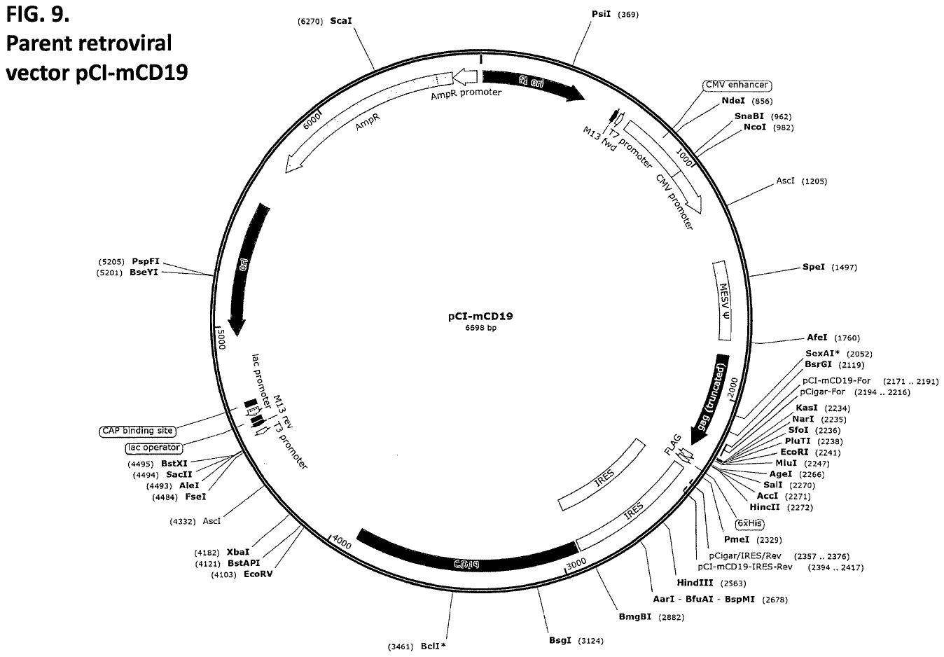

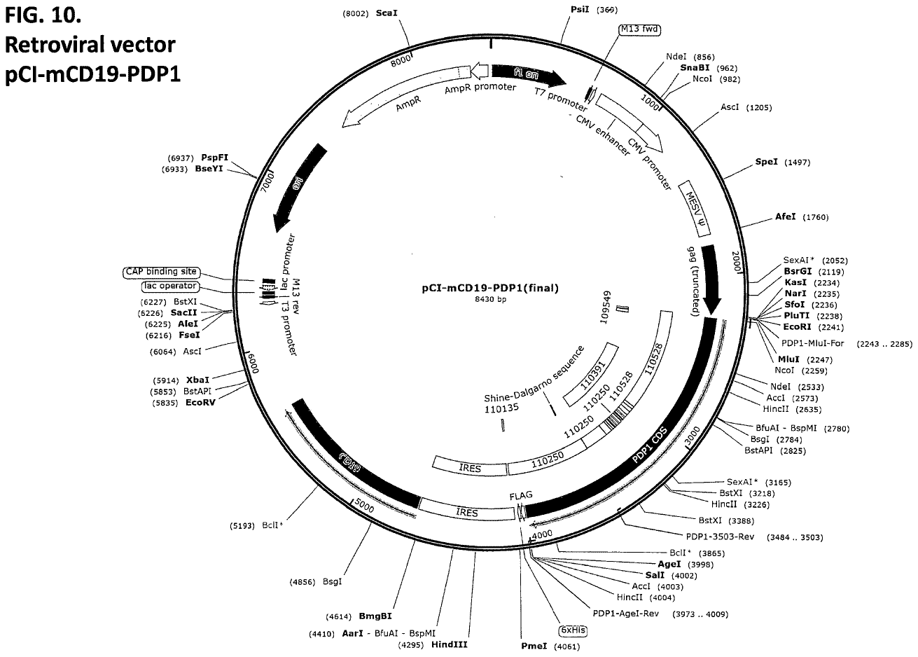

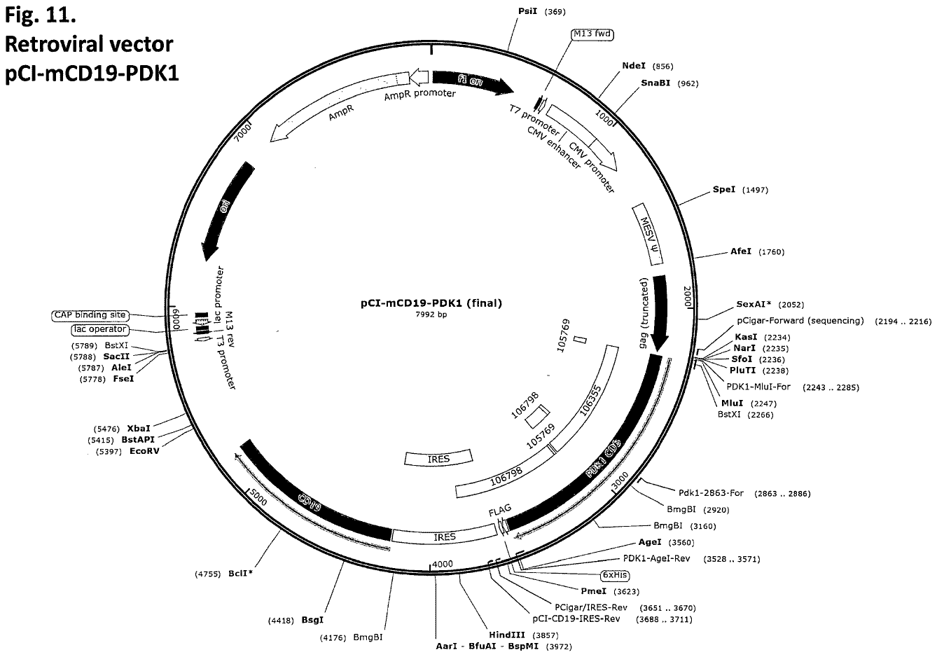

In order to provide T cells with stable enhanced expression of the PDP1 and PDK1 genes, coding sequences for the mouse Pdp1 and Pdk1 enzymes were cloned into a pCl-mCD19 retroviral vector (shown diagrammed in FIG. 9). The resulting vectors pCl-mCD19-PDP1 and pCl-mCD19-PDK1 (shown diagrammed in FIGS. 10 and 11, respectively) and the parental vector were used to generate recombinant retroviruses.

To generate engineered cells containing the genomes of the pCl-mCD19, pCl-mCD19-PDP1 and pCl-mCD19-PDK1 vectors, CD8+ T cells were harvested from the spleens of C57BL/6 (Charles River) mice of approximately 16-weeks of age. CD8+ T cells were magnetically purified using EasySep Mouse CD8+ T cell Isolation kits (STEMCELL™). CD8+ T cells were activated by anti-CD3 and -CD28 antibodies plus recombinant human IL-2 for two days. Then approximately 6 million cells were plated per well on 24-well culture plates and the media was replaced with retrovirus-enriched media. The cells were spun down by centrifugation for 90 minutes at 32° C. and were incubated at 32° C. with recombinant retroviruses encoding the genes of interest for an additional 30 minutes. After transduction, the cells received IL-2 at a final concentration of 25U/mL directly into the retrovirus-containing media before overnight culture. The next day, transduced cells were magnetically purified using BioLegend MojoSort Mouse anti-APC Nanobeads kits with anti-mouse CD19-APC antibody. Cells were cultured for 48 hours at 37° C. before being harvested and counted.

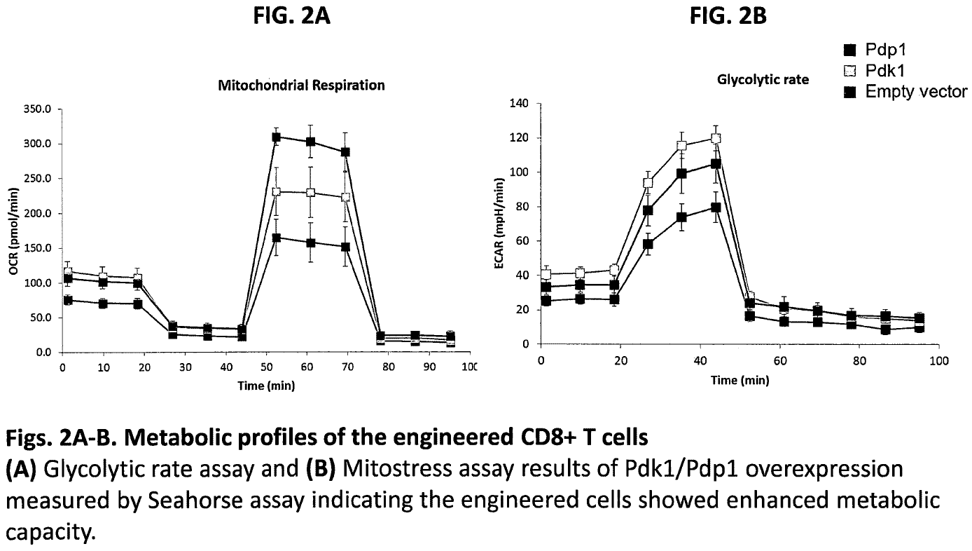

We found retrovirally mediated expression of either Pdp1 or Pdk1 increased both mitochondrial respiration (measured by the oxygen consumption rate (OCR) using a Seahorse bioanalyzer FIG. 2 left panel) and glycolysis (measured by the extracellular acidification rate (ECAR), also using a Seahorse Bioanalyzer, FIG. 2 right panel).

### Example 2: Metabolic Profiles of Engineered T Cells

Seahorse analysis. The Seahorse XFe96 Analyzer (Agilent) instrument was used. This instrument measures changes in extracellular pH and oxygen levels. Since glycolysis is accompanied by secretion of lactic acid, which lowers pH, and oxidative phosphorylation is accompanied by oxygen consumption, this instrument can detect the magnitude of both pathways. For the consumables, Seahorse XFe96 Cell Culture Microplates, Seahorse XFe96 Extracellular Flux Assay Kits, and Seahorse XF Cell Mito stress Test Kit (Agilent) were used. Assays were performed according to the manufacturer's protocols. 150,000 cells were seeded per well for IL-2/IL-15 in vitro differentiated CD8+ T cells. 200,000 cells were seeded per well for ex vivo CD8+ T cells. 1 μM oligomycin, 1.5 μM FCCP and 0.5 μM R/AA were used for mitochondrial stress assays (Seahorse XF Cell Mito Stress Test Kit; Seahorse Agilent cat:103015-100); 0.5 M Rotenone/Antimycin A and 50 mM 2-Deoxyglucose were used for Glycolytic rate assays (Seahorse XF Glycolytic rate Assay; Seahorse Agilent cat:103344-100). Error bars show standard deviation of 6-8 wells for each point on the graphs.

Metabolic analysis using the Seahorse system is a multistep process in which the effect of multiple inhibitors on the cellular oxygen consumption rate (OCR) and extracellular acidification rate (ECAR) are measured.

Looking at FIG. 2A, which indicates OCR, the initial slope of the line indicates basal metabolism. Addition of oligomycin inhibits ATP generation by and proton transit through Complex V of the oxidative phosphorylation chain. This decreases OCR and causes a hyperpolarization of the inner mitochondrial membrane. Addition of carbonyl cyanide-4-(trifluoromethoxy)-phenylhydrazone (FCCP) decouples oxygen consumption from ATP generation, maximizing OCR and revealing the cells peak respiratory capacity. Addition of rotenone and antimycin A (Rot/AA) stops mitochondrial respiration completely, and OCR decreases to a basal level.

Looking at FIG. 2B, which indicates ECAR, the initial slope indicates basal metabolism, comprising acidification from glycolysis as well as the TCA cycle and other sources. Addition of rotenone, which blocks production of ATP by the mitochondria, causes the cells to maximize glycolysis and ECAR. Addition of 2-deoxyglucose inhibits glycolysis, and reduces ECAR to its non-glycolytic level.

Surprisingly, we found retrovirally mediated expression of either Pdp1 or Pdk1 increased both mitochondrial respiration (measured by the oxygen consumption rate (OCR) using a Seahorse bioanalyzer FIG. 2A) and glycolysis (measured by the extracellular acidification rate (ECAR), also using a Seahorse Bioanalyzer, FIG. 2B). Pdp1 had a greater effect on mitochondrial respiration and Pdk1 had a larger effect on glycolysis.

This result indicates that the method of the invention successfully increases the metabolism of the engineered cells.

### Example 3: Response of Engineered T Cells to Antigenic Challenge

OT-1 mice were used for the mouse studies described herein. As used herein, “OT-1 mice” refers to mice containing transgenic inserts for mouse Tcra-V2 and Tcrb-V5 genes encoding a transgenic T cell receptor which recognizes ovalbumin peptide residues 257-264 (OVA257-264) in the context of H2Kb (CD8+co-receptor interaction with MHC class 1). This results in MHC class I-restricted, ovalbumin-specific, CD8+ T cells (referred to herein as “OT-1 cells”). That is, the CD8+ T cells of this mouse primarily recognize OVA257-264 when presented by the MHC I molecule. Immune response dynamics can be studied by in vivo adoptive transfer or in vitro co-culture with cells transgenic for ovalbumin or by direct administration of ovalbumin. OT-1 mice are suitable for the study of CD8+ T cell response to antigen, positive selection, and for any research requiring CD8+ T cells of defined specificity. Like most TCR transgenic mice, OT-1 mice are somewhat immunodeficient. Within this disclosure, OT-1 mice and OT-1 cells which have not been further genetically modified are referred to as wild-type, e.g., “WT OT-I” mice and cells, respectively.

To assess the response of CD8+ cells to a stimulation in the context of an infection, female C57BL.6 mice were infected with either murine gammaherpesvirus-68 (MHV-68-ova) or an attenuated strain of Listeria monocytogenes (LM-ActA-ova). Both pathogens express ovalbumin, thus stimulating OT-1 cells bearing a TCR specific for OVA257-264 during infection.

For MHV-68-ova, mice were infected intranasally for 4000 PFU/mouse. Virus stocks were diluted with PBS to achieve a concentration of 4000 PFU per 30 uL, which was administered intranasally to anesthetized mice (see Smith, et al. (2006), European Journal of Immunology, 36(12), 3186-3197).

LM-ActA-ova was injected retro-orbitally with 1 million CFU/mouse. Bacteria were diluted in PBS to a concentration of 1×10⁶ CFU per 100 uL before injection intravenously via the retro-orbital route to anesthetized mice (Tvinnereim, et al. (2002), Infection and immunity, 70(1), 153-162).

Four days after MHV-68-ova infection and one day after LM-ActA-ova infection, CD8+ T cells from OT-1 mice which had been transduced with retroviruses generated from either the parent (empty) pCI-mCD19 vector, pCl-mCD19-PDP1, or pCl-mCD19-PDK1.

CD8+ T cells were obtained and transduced with retroviral vectors as described in Example 1, except the cells were obtained from CD45.1+ female OT-1 mice. After transduction and enrichment using mouse CD19-APC antibody, cells were cultured for 48 hours at 37° C. Next, the cells were harvested from the cultures and washed with PBS. The cells were resuspended in PBS at a concentration of 20,000 cells/100 uL, and were then injected intravenously into mice by the retro-orbital route into female C57BL.6 mice.

The response of the OT-1 CD8+ cells to infection and specific antigen stimulation was analyzed by flow cytometry. Lung, spleen, and liver tissue was harvested from the infected mice

Cells were not purified for the flow cytometry assays. Total splenocytes were used for flow cytometry, and lung cells were isolated by Percoll gradient separation before staining and flow cytometry analysis.

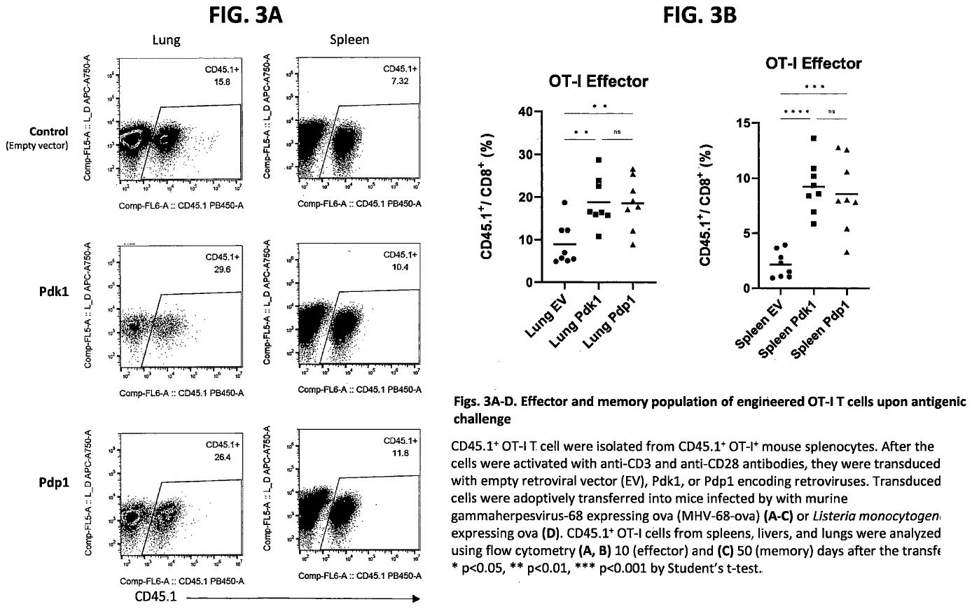

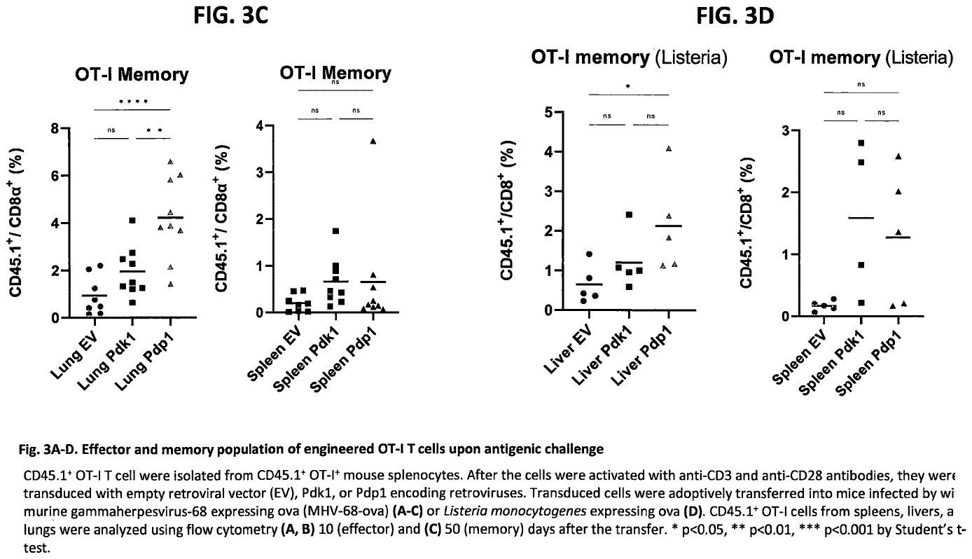

Antibodies used for flow cytometry analysis in FIG. 3-8 include (all anti-mouse): CD8-BV510, CD8α-, CD8β-PE, Live/dead near-IR dye, CD45.1-PE, CD45.1-BV421, CD127-APC, KLRG1-BV421, Ki-67-APC, Annexin V-PE, 7-AAD, TNFα-FITC, IFNγ-APC, Granzyme B-PE/Cγ7,CD62L-BV510, CD103-APC, and CD69-PE/Cγ7.

For the gating, samples were first gated on forward and side scatter to identify lymphocyte-sized cells, then gated for singlets using forward scatter height vs area, then gated on CD8+ dead stain-cells to restrict our analysis to live CD8+ T cells. Among the live CD8+ cells, CD45.1+ cells were gated to analyze the downstream marker phenotypes (CD127, KLRG1, Ki-67, 7-AAD, Annexin V, TNFα, IFNγ, Granzyme B, CD62L, CD69, CD103).

The results were analyzed by one-way ANOVA and Tukey's test.

Early (effector) responses were analyzed using samples from mice 10 days after infection. Memory responses were analyzed using samples from mice 50 days post infection. As can be seen in FIG. 3A, (cell proportions) and FIG. 3B (statistical comparison), larger populations of OT-1 cells transduced with Pdp1 or Pdk1 were observed in the lungs and spleen of MHV-68-ova-infected mice when compared with Tcells transduced with an empty vector control. FIG. 3A shows representative staining and FIG. 3B statistical comparison of the groups. FIG. 3C shows results from 50 days post-infection, indicative of a memory response. Higher frequencies of Pdp1 transduced memory OT-1 cells were detected in the lungs after MHV-68-ova infection. Similar results for a memory response were seen for LM-ActA-ova-infected mice (FIG. 3D). OT-1 T cells transduced with Pdp1 but not Pdk1 were more common in the livers of mice after 50 days compared to those infected with the parent (empty) vector.

Therefore, both Pdp1 and Pdk1 expression leads to increased effector T cell populations and Pdp-1 expression leads to enhanced memory populations, in at least some tissues.

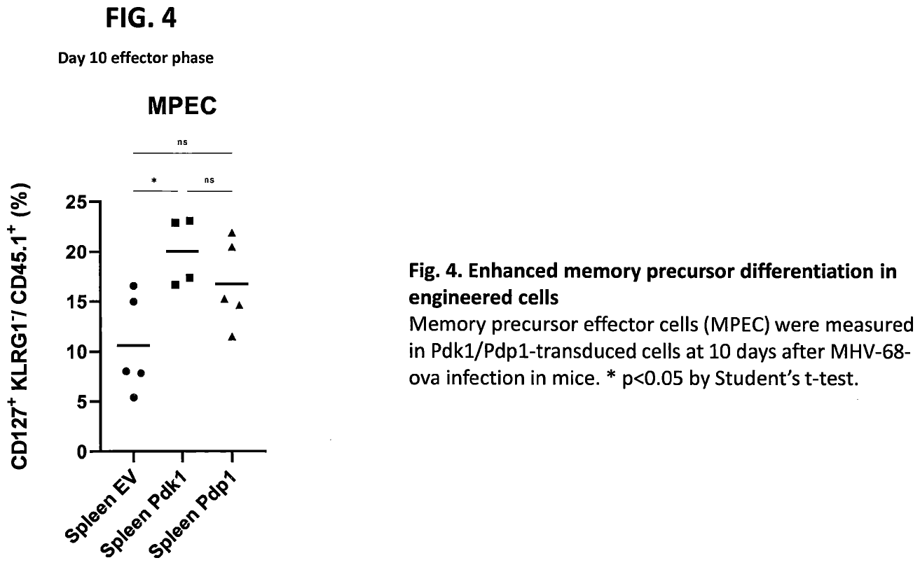

Interestingly, as is shown in FIG. 4, expression of Pdk1, but not Pdp1 resulted in a statistically significant increase in CD127+ KLRG-1-memory CD8 T cell precursors, compared to cells transduced with the empty vector. This demonstrates that Pdk1 expression can also promote differentiation of CD8 T cells toward a memory phenotype.

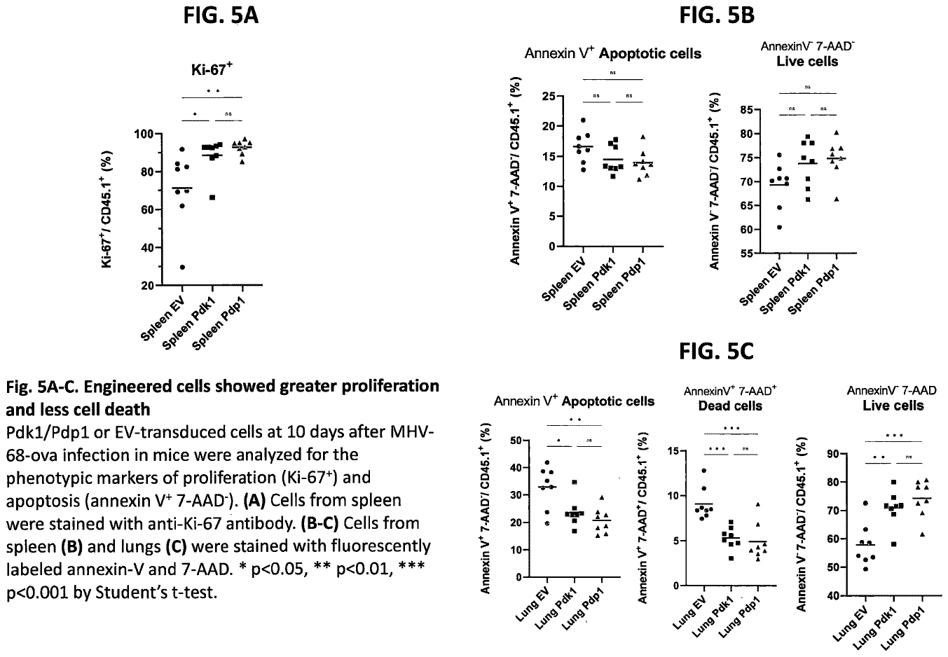

To analyze the reasons why Pdp1 or Pdk1 expression led to increased effector responses, we measured markers of cell proliferation or cell death and apoptosis. Ki67 is a nuclear protein associated with proliferating cells. 7-Aminoactinomycin D (7-AAD) is a fluorescent DNA-binding dye that cannot cross the membrane and bind to the DNA of intact (live) cells. Annexin-V binds to phosphatidylserine, which is a marker of apoptosis when it is exposed on the outer leaflet of the cell membrane. A larger proportion of CD8 T cells transduced with either Pdp1 or Pdk1 stained positive for Ki67, indicating a higher proportion of cells was undergoing proliferation in these groups (FIG. 5A). While no differences in the proportions of live or apoptotic cells were observed in the spleen (FIG. 5B), both Pdk1 and Pdp1 groups had larger populations of live cells and smaller populations of apoptotic or dead cells in the lungs (FIG. 5C), compared with empty vector controls. Therefore, both increased proliferation and decreased cell death contribute to larger effector populations after retrovirally-mediated expression of Pdp1 or Pdk1.

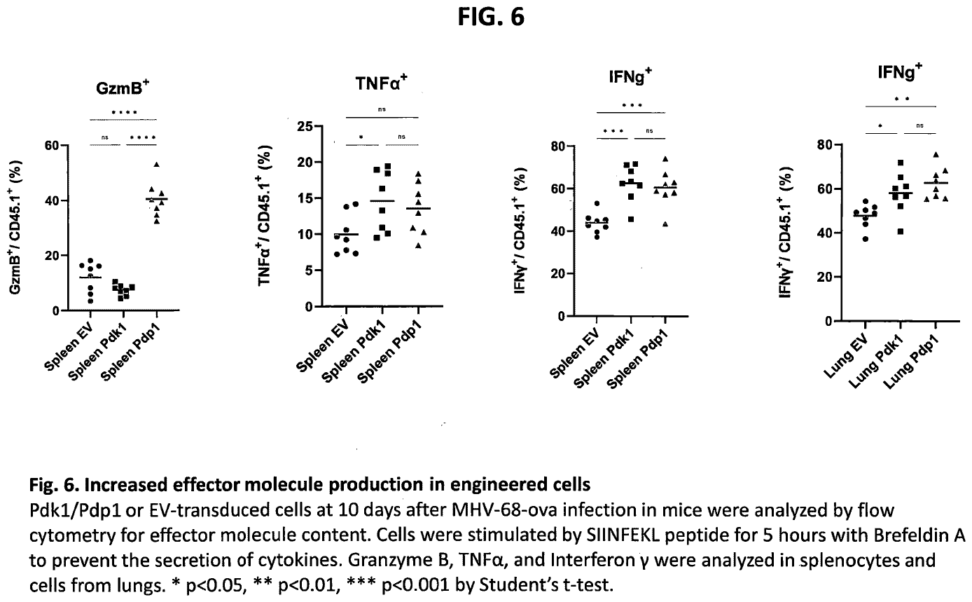

To assess the effect of Pdp1 or Pdk1 expression on the function of transduced T cells, we analyzed the response of CD8+ T cells from mice infected for 10 days with MHV-69-ova to stimulation. Cells were stimulated by SIINFEKL peptide for 5 hours with Brefeldin A to prevent the secretion of cytokines. Granzyme B, TNFα, and Interferon γ were analyzed in splenocytes and cells from lungs. Expression of the CD8T cell effector molecule granzyme B was increased in Pdp1 expressing CD8Tcells in the spleen during the effector response, in addition to interferon gamma (IFNg) in both the spleen and lung (FIG. 6). CD8 T cells expressing Pdk1 showed increased TNF-α and IFN-g in the spleen in addition to IFN-g in the lung, compared with empty vector controls (FIG. 6). Therefore, effector functions are enhanced upon enforced expression of Pdp1 or Pdk1 in CD8 T cells.

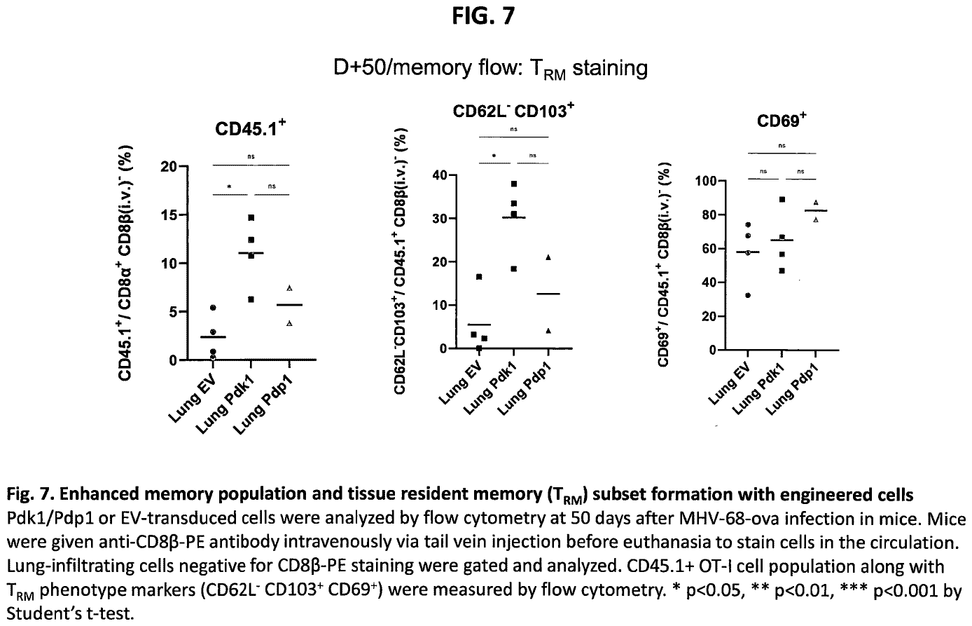

Resident memory CD8 T cells are a population of T cells that do not recirculate but instead stably reside in peripheral tissues and are important in mediating rapid protection against infection and tumors. Resident memory CD8 cells were analyzed in mice at 50 days post MHV-68-ova infection. By intravenously injecting anti-CD8b antibodies into mice prior to euthanasia, circulating memory cells (stained with antibody) can be distinguished from non-circulating tissue resident memory populations (not stained with antibody due to slow diffusion of the antibody into tissue). Pdk1 expressing CD8 T cells had significantly larger populations of resident populations in the lung, as measured by lack of staining with intravenous antibody and the CD62L-CD103+ phenotype indicative of resident memory cells (FIG. 7). Therefore, enforced Pdk1 expression promotes resident memory populations in vivo.

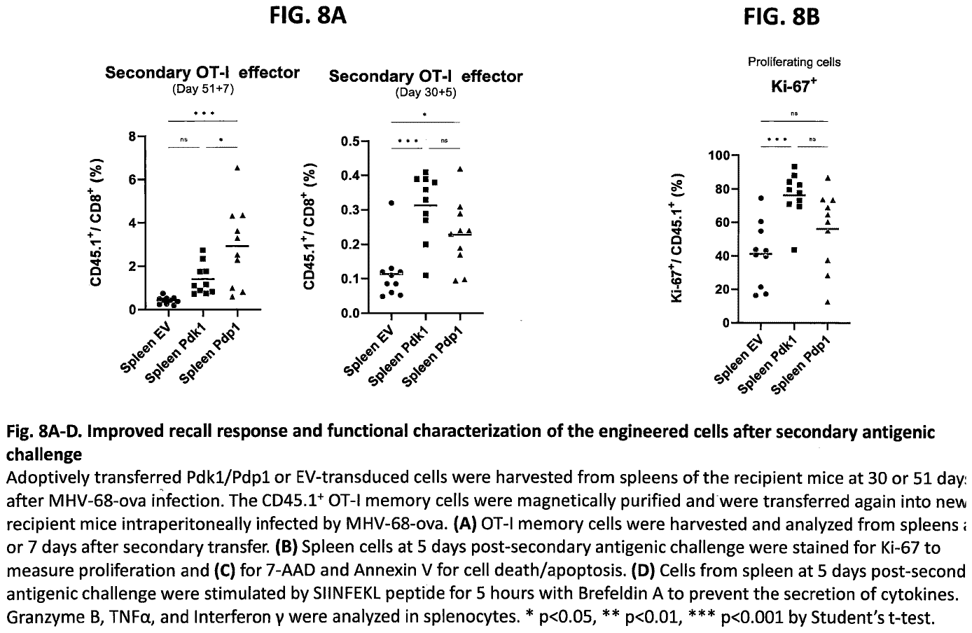

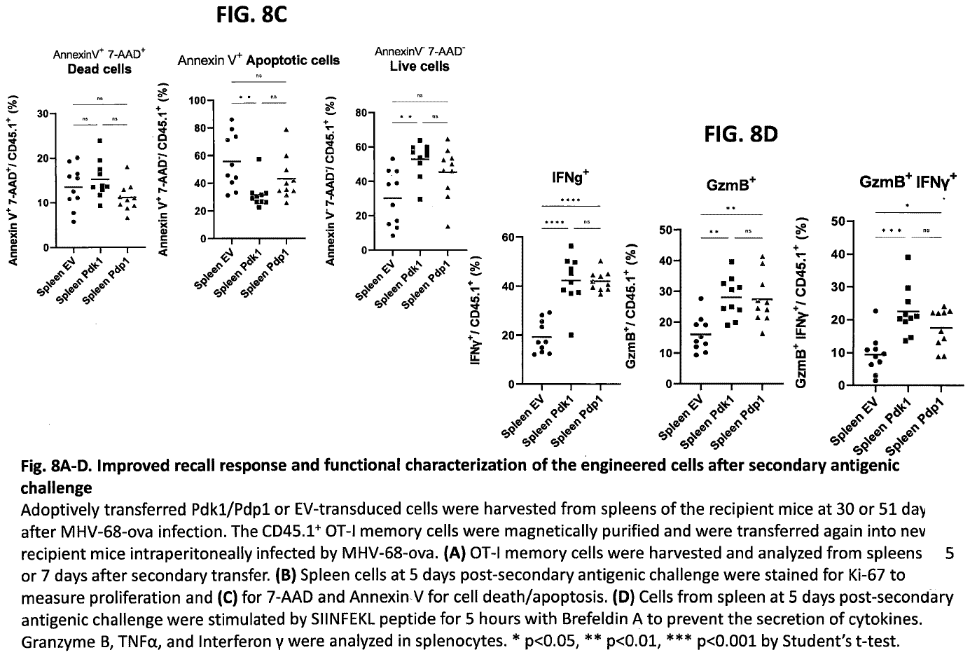

An important characteristic of memory CD8+ T cells is their ability to respond rapidly upon re-exposure to the same antigen with which they were primed. To test if the ability to expand upon antigen rechallenge was affected by Pdp1 or Pdk1 expression, we purified memory CD8+ OT-I cells from mice treated as described in FIG. 3 then adoptively transferred them (20,000 cells, intravenously) to a second group of (female, C57BL.6) mice, which were infected with MHV-68-ova the previous day. Whether memory cells were transferred at 30 or 51 days, OT-I T cells expressing Pdp1 or Pdk1 expanded to larger proportions of the CD8+ population when compared with empty vector controls (FIG. 8A). In Pdk1 expressing cells there were higher proportions of Ki67 expressing cells, indicating more proliferation (FIG. 8B), lower proportions of apoptotic cells and higher proportions of live cells (FIG. 8C), indicative of enhanced CD8 T cell survival. A higher proportion of both Pdk1 or Pdp1 expressing cells produced the effector molecules IFN-g or Granzyme B (FIG. 8D) after the recall response, and in the case of Pdk1 expression, a higher proportion of cells expressing both molecules were present, indicative of polyfunctional effector cells. Therefore, both Pdp1 and Pdk1 enhance the secondary response of memory CD8+ T cells.

### Example 4: Adoptive Cell Therapy with Engineered T Cell in a Human Subject

Immune cells are obtained from a human subject having at least one cancer. The immune cells are preferably T cells obtained from the subject, e.g., from the subject's peripheral blood mononuclear cells obtained via phlebotomy or apheresis. The T cells can be further selected for the presence or absence of one or more markers, such as CD8+/CD45RA+(e.g., naïve CD8+ T cells) or CD8+/CD45RO+(e.g., antigen-experienced CD8+ T cells). The subject optionally undergoes a lymphodepletion procedure, which can include low-dose total body irradiation, chemotherapy such as cyclophosphamide and/or fludarabine, and/or hematopoietic stem cell transplantation, after the T cells are obtained from the subject and prior to reinfusion of the modified T cells into the subject. The T cells are modified ex vivo to express a PDP or PDK transgene using one or more of several approaches described below. The T cells are optionally cultured and expanded ex vivo prior to, simultaneously with, and/or after being modified. The T cells may also be cryopreserved prior to and/or after being modified and subsequently thawed prior to being administered to the subject.

A nucleic acid encoding PDP or PDK is delivered to the T cells using any technique for delivering nucleic acids to mammalian cells, such as use of a retroviral vector, a lentiviral vector, cationic lipids, viral particles, electroporation, and microinjection. The nucleic acid comprises a nucleotide sequence which is at least 75% identical, at least 80% identical, at least 85% identical, at least 90% identical, at least 95% identical, at least 98% identical, or at least 99% identical to SEQ ID NO: 6 or SEQ ID NO. 7. The nucleic acid also comprises promoter(s), enhancer(s), intron(s), 5′ untranslated regions, ribosome binding site(s), 3′ untranslated regions, polyadenylation signal(s), and other accessory sequences necessary for efficient expression.,

The T cells are optionally further modified to express an exogenous TCR or a CAR. The T cells are optionally further modified to express a cytokine, a chemokine, a cytokine receptor, a chemokine receptor, an immune checkpoint inhibitor or antagonist protein. The T cells are optionally further modified to express the exogenous TCR or the CAR prior to or after the T cells are modified to express Pdp1 and/or Pdk1. A nucleic acid encoding an exogenous TCR or a CAR, such as a lentiviral construct, can be delivered to the cells. Any genetic engineering technique can be used to further modify the T cells. For example, the genetic engineering approach is selected from a CRISPR/Cas-based genetic engineering method, a TALEN-based genetic engineering method, a zinc finger-nuclease genetic engineering method, and a transposon-based genetic engineering method. In the case of an exogenous TCR, the cells may also be engineered to express exogenous CD3 gamma, delta, epsilon and zeta genes.

The subject optionally receives an additional cancer therapy prior to, simultaneously with, and/or after reinfusion of the engineered T cells. The optional additional cancer therapy is selected from immunotherapy, chemotherapy, targeted therapy, stem cell transplant, radiation, surgery, and hormone therapy. The optional immunotherapy is selected from immune checkpoint inhibitors (e.g., negative checkpoint blockade), monoclonal antibodies, cancer vaccines, immune system modulators, and adoptive cell therapies including CAR T-cell therapy, exogenous TCR therapy, and TIL therapy.

An effective amount of the engineered T cells is then administered to the subject. Monitoring of peripheral and/or intratumoral T cells in the subject reveals that the engineered T cells expand and persist more than unmodified T cells, due to their increased metabolism, greater expression of proliferation markers, and lower rate of apoptosis and death. The amount of cancer cells in the subject is reduced and/or eliminated following administration of the engineered T cells into the subject in comparison to a subject administered unmodified T cells.

Exemplary Sequences

| SEQ ID NO. | Sequence Description |
|---|---|
| 1 | pCI-mCD19 retroviral vector DNA sequence |
| | GTGCCACCTGACGCGCCCTGTAGCGGCGCATTAAGCGCGGCGGGTGTGGTGGTTA |
| | CGCGCAGCGTGACCGCTACACTTGCCAGCGCCCTAGCGCCCGCTCCTTTCGCTTTCT |
| | TCCCTTCCTTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAAACTTGATT |
| | AGGGTGATGGTTCACGTAGTGGGCCATCGCCCTGATAGACGGTTTTTCGCCCTTTG |
| | ACGTTGGAGTCCACGTTCTTTAATAGTGGACTCTTGTTCCAAACTGGAACAACACTC |
| | AACCCTATCTCGGTCTATTCTTTTGATTTATAAGGGATTTTGCCGATTTCGGCCTATT |
| | GGTTAAAAAATGAGCTGATTTAACAAAAATTTAACGCGAATTTTAACAAAATATTA |
| | ACGCTTACAATTTCCATTCGCCATTCAGGCTGCGCAACTGTTGGGAAGGGCGATCG |
| | GTGCGGGCCTCTTCGCTATTACGCCAGCTGGCGAAAGGGGGATGTGCTGCAAGGC |
| | GATTAAGTTGGGTAACGCCAGGGTTTTCCCAGTCACGACGTTGTAAAACGACGGC |
| | CAGTGAGCGCGCGTAATACGACTCACTATAGGGCGAATTGGGTACCGGGCCCCCC |
| | CTCGACCGCGTTACATAACTTACGGTAAATGGCCCGCCTGGCTGACCGCCCAACGA |
| | CCCCCGCCCATTGACGTCAATAATGACGTATGTTCCCATAGTAACGCCAATAGGGA |
| | CTTTCCATTGACGTCAATGGGTGGAGTATTTACGGTAAACTGCCCACTTGGCAGTA |
| | CATCAAGTGTATCATATGCCAAGTACGCCCCCTATTGACGTCAATGACGGTAAATG |
| | GCCCGCCTGGCATTATGCCCAGTACATGACCTTATGGGACTTTCCTACTTGGCAGTA |
| | CATCTACGTATTAGTCATCGCTATTACCATGGTGATGCGGTTTTGGCAGTACATCAA |
| | TGGGCGTGGATAGCGGTTTGACTCACGGGGATTTCCAAGTCTCCACCCCATTGACG |
| | TCAATGGGAGTTTGTTTTGGCACCAAAATCAACGGGACTTTCCAAAATGTCGTAAC |
| | AACTCCGCCCCATTGACGCAAATGGGCGGTAGGCGTGTACGGTGGGAGGTCTATA |
| | TAAGCAGAGCTCGTTTAGTGAACCGgcGCGCCAGTCCTCCGATAGACTGCGTCGCC |
| | CGGGTACCCGTATTCCCAATAAAGCCTCTTGCTGTTTGCATCCGAATCGTGGACTCG |
| | CTGATCCTTGGGAGGGTCTCCTCAGATTGATTGACTGCCCACCTCGGGGGTCTTTC |
| | ATTTGGAGGTTCCACCGAGATTTGGAGACCCCTGCCCAGGGACCACCGACCCCCCC |
| | GCCGGGAGGTAAGCTGGCCAGCGGTCGTTTCGTGTCTGTCTCTGTCTTTGTGCGTG |
| | TTTGTGCCGGCATCTAATGTTTGCGCCTGCGTCTGTACTAGTTAGCTAACTAGCTCT |
| | GTATCTGGCGGACCCGTGGTGGAACTGACGAGTTCTGAACACCCGGCCGCAACCC |
| | TGGGAGACGTCCCAGGGACTTTGGGGGCCGTTTTTGTGGCCCGACCTGAGGAAGG |
| | GAGTCGATGTGGAATCCGACCCCGTCAGGATATGTGGTTCTGGTAGGAGACGAGA |
| | ACCTAAAACAGTTCCCGCCTCCGTCTGAATTTTTGCTTTCGGTTTGGAACCGAAGCC |
| | GCGCGTCTTGTCTGCTGCAGCGCTGCAGCATCGTTCTGTGTTGTCTCTGTCTGACTG |
| | TGTTTCTGTATTTGTCTGAAAATTAGGGCCAGACTGTTACCACTCCCTTAAGTTTGA |
| | CCTTAGGTCACTGGAAAGATGTCGAGCGGATCGCTCACAACCAGTCGGTAGATGT |
| | CAAGAAGAGACGTTGGGTTACCTTCTGCTCTGCAGAATGGCCAACCTTTAACGTCG |
| | GATGGCCGCGAGACGGCACCTTTAACCGAGACCTCATCACCCAGGTTAAGATCAA |
| | GGTCTTTTCACCTGGCCCGCATGGACACCCAGACCAGGTCCCCTACATCGTGACCT |
| | GGGAAGCCTTGGCTTTTGACCCCCCTCCCTGGGTCAAGCCCTTTGTACACCCTAAGC |
| | CTCCGCCTCCTCTTCCTCCATCCGCCCCGTCTCTCCCCCTTGAACCTCCTCGTTCGAC |
| | CCCGCCTCGATCCTCCCTTTATCCAGCCCTCACTCCTTCTCTAGGCGCCGgaattcacgc |
| | gtgggcccgGGATCCACCGGTCGACTACAAGGACGACGATGACAAGGGGCATCACCA |
| | TCACCACCATTAGCTAGCGTTTAAACTTCCGCCCCTCTCCCTCCCCCCCCCCTAACGT |
| | TACTGGCCGAAGCCGCTTGGAATAAGGCCGGTGTGCGTTTGTCTATATGTTATTTT |
| | CCACCATATTGCCGTCTTTTGGCAATGTGAGGGCCCGGAAACCTGGCCCTGTCTTCT |
| | TGACGAGCATTCCTAGGGGTCTTTCCCCTCTCGCCAAAGGAATGCAAGGTCTGTTG |
| | AATGTCGTGAAGGAAGCAGTTCCTCTGGAAGCTTCTTGAAGACAAACAACGTCTGT |
| | AGCGACCCTTTGCAGGCAGCGGAACCCCCCACCTGGCGACAGGTGCCTCTGCGGC |
| | CAAAAGCCACGTGTATAAGATACACCTGCAAAGGCGGCACAACCCCAGTGCCACG |
| | TTGTGAGTTGGATAGTTGTGGAAAGAGTCAAATGGCTCTCCTCAAGCGTATTCAAC |
| | AAGGGGCTGAAGGATGCCCAGAAGGTACCCCATTGTATGGGATCTGATCTGGGGC |
| | CTCGGTACACATGCTTTACATGTGTTTAGTCGAGGTTAAAAAAACGTCTAGGCCCC |
| | CCGAACCACGGGGACGTGGTTTTCCTTTGAAAAACACGATGATAATATGGCCACCA |
| | TGCCCTCCCCTCTGCCCGTGAGCTTCCTGCTGTTCCTGACCCTGGTGGGAGGCAGG |
| | CCCCAGAAGAGCCTGCTGGTGGAGGTGGAGGAAGGCGGCAACGTGGTGCTGCCC |
| | TGTCTGCCTGATAGCTCCCCTGTGAGCAGCGAGAAGCTGGCCTGGTACAGGGGCA |
| | ACCAGAGCACCCCTTTCCTGGAACTGAGCCCTGGAAGCCCTGGACTGGGCCTGCAC |
| | GTGGGAAGCCTGGGCATCCTGCTGGTGATCGTGAACGTGTCCGACCACATGGGCG |
| | GCTTCTATCTGTGCCAGAAGAGGCCCCCCTTCAAAGACATCTGGCAGCCCGCCTGG |
| | ACCGTGAACGTGGAGGACAGCGGCGAGATGTTCAGGTGGAACGCCAGCGACGTC |
| | AGGGACCTGGACTGCGACCTGAGAAACAGAAGCTCCGGCTCCCACAGGAGCACAA |
| | GCGGCAGCCAGCTGTACGTGTGGGCTAAGGACCACCCTAAGGTGTGGGGCACAA |
| | AGCCCGTGTGTGCCCCTAGGGGAAGCTCCCTCAACCAGAGCCTGATCAACCAGGA |
| | CCTGACAGTGGCCCCCGGAAGCACCCTGTGGCTGAGCTGTGGCGTGCCTCCTGTG |
| | CCTGTGGCCAAAGGATCCATCTCCTGGACCCACGTGCACCCCAGAAGGCCCAACGT |
| | GAGCCTGCTGAGCCTGAGCCTGGGAGGCGAACACCCCGTGAGGGAGATGTGGGT |
| | GTGGGGCAGCCTGCTGCTGCTGCCTCAGGCTACCGCCCTGGACGAAGGCACCTAC |
| | TACTGTCTGAGGGGCAACCTGACAATCGAGAGGCACGTGAAGGTGATCGCTAGAA |
| | GCGCCGTGTGGCTGTGGCTGCTGAGGACCGGCGGATGGATTGTGCCCGTGGTGA |
| | CCCTGGTGTACGTGATCTTCTGCATGGTGTCCCTGGTGGCCTTCCTGTACTGTCAGA |
| | GAGCCTTCATCCTCAGGAGAAAGAGGAAGAGGATGACATAGTGAttaacgcggccAA |
| | TTCGATAAAATAAAAGATTTTATTTAGTCTCCAGAAAAAGGGGGGAATGAAAGAC |
| | CCCACCTGTAGGTTTGGCAAGCTAGCTTAAGTAACGCCATTTTGCAAGGCATGGAA |
| | AATACATAACTGAGAATAGAGAAGTTCAGATCAAGGTTAGGAACAGAGAGACAGC |
| | AGAATATGGGCCAAACAGGATATCTGTGGTAAGCAGTTCCTGCCCCGGCTCAGGG |
| | CCAAGAACAGATGGTCCCCAGATGCGGTCCCGCCCTCAGCAGTTTCTAGAGAACCA |
| | TCAGATGTTTCCAGGGTGCCCCAAGGACCTGAAATGACCCTGTGCCTTATTTGAAC |
| | TAACCAATCAGTTCGCTTCTCGCTTCTGTTCGCGCGCTTCTGCTCCCCGAGCTCAAT |
| | AAAAGAGCCCACAACCCCTCACTCGGCGCGCCAGTCCTCCGATAGACTGCGTCGCC |
| | CGGGTACCCGTGTATCCAATAAACCCTCTTGCAGTTGCATCCGACTTGTGGTCTCGC |
| | TGTTCCTTGGGAGGGTCTCCTCTGAGTGATTGACTACCCGTCAGCGGGGGTCTTTC |
| | AGCGGCCGGCCGCCACCGCGGTGGAGCTCCAGCTTTTGTTCCCTTTAGTGAGGGTT |
| | AATTGCGCGCTTGGCGTAATCATGGTCATAGCTGTTTCCTGTGTGAAATTGTTATCC |
| | GCTCACAATTCCACACAACATACGAGCCGGAAGCATAAAGTGTAAAGCCTGGGGT |
| | GCCTAATGAGTGAGCTAACTCACATTAATTGCGTTGCGCTCACTGCCCGCTTTCCAG |
| | TCGGGAAACCTGTCGTGCCAGCTGCATTAATGAATCGGCCAACGCGCGGGGAGAG |
| | GCGGTTTGCGTATTGGGCGCTCTTCCGCTTCCTCGCTCACTGACTCGCTGCGCTCGG |
| | TCGTTCGGCTGCGGCGAGCGGTATCAGCTCACTCAAAGGCGGTAATACGGTTATCC |
| | ACAGAATCAGGGGATAACGCAGGAAAGAACATGTGAGCAAAAGGCCAGCAAAAG |
| | GCCAGGAACCGTAAAAAGGCCGCGTTGCTGGCGTTTTTCCATAGGCTCCGCCCCCC |
| | TGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCCG |
| | ACCCTGCCGCTTACCGGATACCTGTCCGCCTTTCTCCCTTCGGGAAGCGTGGCGCTT |
| | TCTCATAGCTCACGCTGTAGGTATCTCAGTTCGGTGTAGGTCGTTCGCTCCAAGCT |
| | GGGCTGTGTGCACGAACCCCCCGTTCAGCCCGACCGCTGCGCCTTATCCGGTAACT |
| | ATCGTCTTGAGTCCAACCCGGTAAGACACGACTTATCGCCACTGGCAGCAGCCACT |
| | GGTAACAGGATTAGCAGAGCGAGGTATGTAGGCGGTGCTACAGAGTTCTTGAAGT |
| | GGTGGCCTAACTACGGCTACACTAGAAGGACAGTATTTGGTATCTGCGCTCTGCTG |
| | AAGCCAGTTACCTTCGGAAAAAGAGTTGGTAGCTCTTGATCCGGCAAACAAACCAC |
| | CGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTGTCTAT |
| | TTCGTTCATCCATAGTTGCCTGACTCCCCGTCGTGTAGATAACTACGATACGGGAG |
| | GGCTTACCATCTGGCCCCAGTGCTGCAATGATACCGCGAGACCCACGCTCACCGGC |
| | TCCAGATTTATCAGCAATAAACCAGCCAGCCGGAAGGGCCGAGCGCAGAAGTGGT |
| | CCTGCAACTTTATCCGCCTCCATCCAGTCTATTAATTGTTGCCGGGAAGCTAGAGTA |
| | AGTAGTTCGCCAGTTAATAGTTTGCGCAACGTTGTTGCCATTGCTACAGGCATCGT |
| | GGTGTCACGCTCGTCGTTTGGTATGGCTTCATTCAGCTCCGGTTCCCAACGATCAA |
| | GGCGAGTTACATGATCCCCCATGTTGTGCAAAAAAGCGGTTAGCTCCTTCGGTCCT |
| | CCGATCGTTGTCAGAAGTAAGTTGGCCGCAGTGTTATCACTCATGGTTATGGCAGC |
| | ACTGCATAATTCTCTTACTGTCATGCCATCCGTAAGATGCTTTTCTGTGACTGGTGA |
| | GTACTCAACCAAGTCATTCTGAGAATAGTGTATGCGGCGACCGAGTTGCTCTTGCC |
| | CGGCGTCAATACGGGATAATACCGCGCCACATAGCAGAACTTTAAAAGTGCTCATC |
| | ATTGGAAAACGTTCTTCGGGGCGAAAACTCTCAAGGATCTTACCGCTGTTGAGATC |
| | CAGTTCGATGTAACCCACTCGTGCACCCAACTGATCTTCAGCATCTTTTACTTTCACC |
| | AGCGTTTCTGGGTGAGCAAAAACAGGAAGGCAAAATGCCGCAAAAAAGGGAATA |
| | AGGGCGACACGGAAATGTTGAATACTCATACTCTTCCTTTTTCAATATTATTGAAGC |
| | ATTTATCAGGGTTATTGTCTCATGAGCGGATACATATTTGAATGTATTTAGAAAAAT |
| | AAACAAATAGGGGTTCCGCGCACATTTCCCCGAAAA |
| 2 | pCI-mCD19-PDP1 retroviral vector DNA sequence |
| | GTGCCACCTGACGCGCCCTGTAGCGGCGCATTAAGCGCGGCGGGTGTGGTGGTTA |
| | CGCGCAGCGTGACCGCTACACTTGCCAGCGCCCTAGCGCCCGCTCCTTTCGCTTTCT |
| | TCCCTTCCTTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAAACTTGATT |
| | AGGGTGATGGTTCACGTAGTGGGCCATCGCCCTGATAGACGGTTTTTCGCCCTTTG |
| | ACGTTGGAGTCCACGTTCTTTAATAGTGGACTCTTGTTCCAAACTGGAACAACACTC |
| | AACCCTATCTCGGTCTATTCTTTTGATTTATAAGGGATTTTGCCGATTTCGGCCTATT |
| | GGTTAAAAAATGAGCTGATTTAACAAAAATTTAACGCGAATTTTAACAAAATATTA |
| | ACGCTTACAATTTCCATTCGCCATTCAGGCTGCGCAACTGTTGGGAAGGGCGATCG |
| | GTGCGGGCCTCTTCGCTATTACGCCAGCTGGCGAAAGGGGGATGTGCTGCAAGGC |
| | GATTAAGTTGGGTAACGCCAGGGTTTTCCCAGTCACGACGTTGTAAAACGACGGC |
| | CAGTGAGCGCGCGTAATACGACTCACTATAGGGCGAATTGGGTACCGGGCCCCCC |
| | CTCGACCGCGTTACATAACTTACGGTAAATGGCCCGCCTGGCTGACCGCCCAACGA |
| | CCCCCGCCCATTGACGTCAATAATGACGTATGTTCCCATAGTAACGCCAATAGGGA |
| | CTTTCCATTGACGTCAATGGGTGGAGTATTTACGGTAAACTGCCCACTTGGCAGTA |
| | CATCAAGTGTATCATATGCCAAGTACGCCCCCTATTGACGTCAATGACGGTAAATG |
| | GCCCGCCTGGCATTATGCCCAGTACATGACCTTATGGGACTTTCCTACTTGGCAGTA |
| | CATCTACGTATTAGTCATCGCTATTACCATGGTGATGCGGTTTTGGCAGTACATCAA |
| | TGGGCGTGGATAGCGGTTTGACTCACGGGGATTTCCAAGTCTCCACCCCATTGACG |
| | TCAATGGGAGTTTGTTTTGGCACCAAAATCAACGGGACTTTCCAAAATGTCGTAAC |
| | AACTCCGCCCCATTGACGCAAATGGGCGGTAGGCGTGTACGGTGGGAGGTCTATA |
| | TAAGCAGAGCTCGTTTAGTGAACCGgcGCGCCAGTCCTCCGATAGACTGCGTCGCC |
| | CGGGTACCCGTATTCCCAATAAAGCCTCTTGCTGTTTGCATCCGAATCGTGGACTCG |
| | CTGATCCTTGGGAGGGTCTCCTCAGATTGATTGACTGCCCACCTCGGGGGTCTTTC |
| | ATTTGGAGGTTCCACCGAGATTTGGAGACCCCTGCCCAGGGACCACCGACCCCCCC |
| | GCCGGGAGGTAAGCTGGCCAGCGGTCGTTTCGTGTCTGTCTCTGTCTTTGTGCGTG |
| | TTTGTGCCGGCATCTAATGTTTGCGCCTGCGTCTGTACTAGTTAGCTAACTAGCTCT |
| | GTATCTGGCGGACCCGTGGTGGAACTGACGAGTTCTGAACACCCGGCCGCAACCC |
| | TGGGAGACGTCCCAGGGACTTTGGGGGCCGTTTTTGTGGCCCGACCTGAGGAAGG |
| | GAGTCGATGTGGAATCCGACCCCGTCAGGATATGTGGTTCTGGTAGGAGACGAGA |
| | ACCTAAAACAGTTCCCGCCTCCGTCTGAATTTTTGCTTTCGGTTTGGAACCGAAGCC |
| | GCGCGTCTTGTCTGCTGCAGCGCTGCAGCATCGTTCTGTGTTGTCTCTGTCTGACTG |
| | TGTTTCTGTATTTGTCTGAAAATTAGGGCCAGACTGTTACCACTCCCTTAAGTTTGA |
| | CCTTAGGTCACTGGAAAGATGTCGAGCGGATCGCTCACAACCAGTCGGTAGATGT |
| | CAAGAAGAGACGTTGGGTTACCTTCTGCTCTGCAGAATGGCCAACCTTTAACGTCG |
| | GATGGCCGCGAGACGGCACCTTTAACCGAGACCTCATCACCCAGGTTAAGATCAA |
| | GGTCTTTTCACCTGGCCCGCATGGACACCCAGACCAGGTCCCCTACATCGTGACCT |
| | GGGAAGCCTTGGCTTTTGACCCCCCTCCCTGGGTCAAGCCCTTTGTACACCCTAAGC |
| | CTCCGCCTCCTCTTCCTCCATCCGCCCCGTCTCTCCCCCTTGAACCTCCTCGTTCGAC |
| | CCCGCCTCGATCCTCCCTTTATCCAGCCCTCACTCCTTCTCTAGGCGCCGgaattcacgc |
| | gtccgccaccatggagcggcggcggtgcgcctgtccgggctcctggagttgcccgccgggggctgtgatgcc |
| | gcctcgcctgccgggaatcccagtcagaacgtccagtctgccactgttctctgatgccatgccagcaccaact |
| | caactgttttttcctctcgtccgtaactgtgaactgagcagaatctatggcactgcatgttactgccaccacaa |
| | acatctctgctgttcaccaccatacattcctcagaatcgtctgaggtacacaccccatcctgcatatgctacctt |
| | ttgtaggccacgggagaactggtggcagtatactcaaggaaggagatacgcttctacaccgcagaaatttta |
| | cctcacacctccacaagtcaacagcatccttaaagctaatgaatacagcttcaaagtaccagaatttgatggc |
| | aaaaatgtcagttccattcttggatttgacagcaatcagctgcctgcaaatgcacccatagaggaccggaga |
| | agtgcagcaacctgcttgcagaccagagggatgctcttgggggtttttgatggtcatgcaggctgtgcttgttc |
| | ccaggcagtcagtgaaagactcttctattatattgctgtttccttgttgccccatgagactttgctagagattga |
| | aaatgcagtggagagtggtcgggcactgctacctatccttcagtggcacaagcaccccaatgattacttcagt |
| | aaggaggcgtccaaattgtatttcaacagcttgaggacttactggcaagaactcatagacctcaatactgga |
| | gaatcagctgatattgatgttaaggaggctttaattaatgctttcaagagactcgataatgacatttcattgga |
| | ggctcaagttggtgatcctaattcttttctaaattacctggtgcttcgggtagcattttctggggctactgcttgt |
| | gtggcccatgtagatggtgttgacctccatgtggctaacactggtgatagtagagccatgctaggtgtgcaag |
| | aagaagatggctcttggtcagcagtcacactctctaatgaccacaatgctcagaatgaaagagaactagag |
| | cgtctgaaactggaacacccaaaaaatgaggccaagagcgtggtaaagcaggaccggctgcttggcttgct |
| | gatgccctttagggcttttggagatgtaaagttcaaatggagcattgaccttcaaaagagagtgatagagtct |
| | ggcccagaccagttgaatgacaatgaatacaccaagtttatccctcctaactatcatacacctccttatcttac |
| | tgctgagccagaggtaacttatcacagattaaggccacaggataaattcctagtgttagcaactgatgggttg |
| | tgggagactatgcatagacaggatgtggttaggattgtgggtgaatacttaactggtatgcatcaccaacagc |
| | caatagcagttggtgggtacaaggtgactctgggacagatgcatggccttttaacagaaaggagagcaaag |
| | atgtcatcagtgtttgaggatcagaatgcagcaacccatctcattcgccatgctgtaggcaataatgaatttg |
| | gggctgttgatcatgaacgactctctaaaatgcttagtcttcctgaagagcttgctcggatgtatagagatgac |
| | attacaatcattgtagttcagttcaattctcatgttgtaggggcataccaaaaccaggaacagtaaACCGG |
| | TCGACTACAAGGACGACGATGACAAGGGGCATCACCATCACCACCATTAGCTAGC |
| | GTTTAAACTTCCGCCCCTCTCCCTCCCCCCCCCCTAACGTTACTGGCCGAAGCCGCTT |
| | GGAATAAGGCCGGTGTGCGTTTGTCTATATGTTATTTTCCACCATATTGCCGTCTTT |
| | TGGCAATGTGAGGGCCCGGAAACCTGGCCCTGTCTTCTTGACGAGCATTCCTAGG |
| | GGTCTTTCCCCTCTCGCCAAAGGAATGCAAGGTCTGTTGAATGTCGTGAAGGAAGC |
| | AGTTCCTCTGGAAGCTTCTTGAAGACAAACAACGTCTGTAGCGACCCTTTGCAGGC |
| | AGCGGAACCCCCCACCTGGCGACAGGTGCCTCTGCGGCCAAAAGCCACGTGTATA |
| | AGATACACCTGCAAAGGCGGCACAACCCCAGTGCCACGTTGTGAGTTGGATAGTT |
| | GTGGAAAGAGTCAAATGGCTCTCCTCAAGCGTATTCAACAAGGGGCTGAAGGATG |
| | CCCAGAAGGTACCCCATTGTATGGGATCTGATCTGGGGCCTCGGTACACATGCTTT |
| | ACATGTGTTTAGTCGAGGTTAAAAAAACGTCTAGGCCCCCCGAACCACGGGGACG |
| | TGGTTTTCCTTTGAAAAACACGATGATAATATGGCCACCATGCCCTCCCCTCTGCCC |
| | GTGAGCTTCCTGCTGTTCCTGACCCTGGTGGGAGGCAGGCCCCAGAAGAGCCTGC |
| | TGGTGGAGGTGGAGGAAGGCGGCAACGTGGTGCTGCCCTGTCTGCCTGATAGCTC |
| | CCCTGTGAGCAGCGAGAAGCTGGCCTGGTACAGGGGCAACCAGAGCACCCCTTTC |
| | CTGGAACTGAGCCCTGGAAGCCCTGGACTGGGCCTGCACGTGGGAAGCCTGGGC |
| | ATCCTGCTGGTGATCGTGAACGTGTCCGACCACATGGGCGGCTTCTATCTGTGCCA |
| | GAAGAGGCCCCCCTTCAAAGACATCTGGCAGCCCGCCTGGACCGTGAACGTGGAG |
| | GACAGCGGCGAGATGTTCAGGTGGAACGCCAGCGACGTCAGGGACCTGGACTGC |
| | GACCTGAGAAACAGAAGCTCCGGCTCCCACAGGAGCACAAGCGGCAGCCAGCTGT |
| | ACGTGTGGGCTAAGGACCACCCTAAGGTGTGGGGCACAAAGCCCGTGTGTGCCCC |
| | TAGGGGAAGCTCCCTCAACCAGAGCCTGATCAACCAGGACCTGACAGTGGCCCCC |
| | GGAAGCACCCTGTGGCTGAGCTGTGGCGTGCCTCCTGTGCCTGTGGCCAAAGGAT |
| | CCATCTCCTGGACCCACGTGCACCCCAGAAGGCCCAACGTGAGCCTGCTGAGCCTG |
| | AGCCTGGGAGGCGAACACCCCGTGAGGGAGATGTGGGTGTGGGGCAGCCTGCTG |
| | CTGCTGCCTCAGGCTACCGCCCTGGACGAAGGCACCTACTACTGTCTGAGGGGCA |
| | ACCTGACAATCGAGAGGCACGTGAAGGTGATCGCTAGAAGCGCCGTGTGGCTGTG |
| | GCTGCTGAGGACCGGCGGATGGATTGTGCCCGTGGTGACCCTGGTGTACGTGATC |
| | TTCTGCATGGTGTCCCTGGTGGCCTTCCTGTACTGTCAGAGAGCCTTCATCCTCAGG |
| | AGAAAGAGGAAGAGGATGACATAGTGAttaacgcggccAATTCGATAAAATAAAAGA |
| | TTTTATTTAGTCTCCAGAAAAAGGGGGGAATGAAAGACCCCACCTGTAGGTTTGGC |
| | AAGCTAGCTTAAGTAACGCCATTTTGCAAGGCATGGAAAATACATAACTGAGAATA |
| | GAGAAGTTCAGATCAAGGTTAGGAACAGAGAGACAGCAGAATATGGGCCAAACA |
| | GGATATCTGTGGTAAGCAGTTCCTGCCCCGGCTCAGGGCCAAGAACAGATGGTCC |
| | CCAGATGCGGTCCCGCCCTCAGCAGTTTCTAGAGAACCATCAGATGTTTCCAGGGT |
| | GCCCCAAGGACCTGAAATGACCCTGTGCCTTATTTGAACTAACCAATCAGTTCGCTT |
| | CTCGCTTCTGTTCGCGCGCTTCTGCTCCCCGAGCTCAATAAAAGAGCCCACAACCCC |
| | TCACTCGGCGCGCCAGTCCTCCGATAGACTGCGTCGCCCGGGTACCCGTGTATCCA |
| | ATAAACCCTCTTGCAGTTGCATCCGACTTGTGGTCTCGCTGTTCCTTGGGAGGGTCT |
| | CCTCTGAGTGATTGACTACCCGTCAGCGGGGGTCTTTCAGCGGCCGGCCGCCACCG |
| | CGGTGGAGCTCCAGCTTTTGTTCCCTTTAGTGAGGGTTAATTGCGCGCTTGGCGTA |
| | ATCATGGTCATAGCTGTTTCCTGTGTGAAATTGTTATCCGCTCACAATTCCACACAA |
| | CATACGAGCCGGAAGCATAAAGTGTAAAGCCTGGGGTGCCTAATGAGTGAGCTAA |
| | CTCACATTAATTGCGTTGCGCTCACTGCCCGCTTTCCAGTCGGGAAACCTGTCGTGC |
| | CAGCTGCATTAATGAATCGGCCAACGCGCGGGGAGAGGCGGTTTGCGTATTGGGC |
| | GCTCTTCCGCTTCCTCGCTCACTGACTCGCTGCGCTCGGTCGTTCGGCTGCGGCGA |
| | GCGGTATCAGCTCACTCAAAGGCGGTAATACGGTTATCCACAGAATCAGGGGATA |
| | ACGCAGGAAAGAACATGTGAGCAAAAGGCCAGCAAAAGGCCAGGAACCGTAAAA |
| | AGGCCGCGTTGCTGGCGTTTTTCCATAGGCTCCGCCCCCCTGACGAGCATCACAAA |
| | AATCGACGCTCAAGTCAGAGGTGGCGAAACCCGACAGGACTATAAAGATACCAGG |
| | CGTTTCCCCCTGGAAGCTCCCTCGTGCGCTCTCCTGTTCCGACCCTGCCGCTTACCG |
| | GATACCTGTCCGCCTTTCTCCCTTCGGGAAGCGTGGCGCTTTCTCATAGCTCACGCT |
| | GTAGGTATCTCAGTTCGGTGTAGGTCGTTCGCTCCAAGCTGGGCTGTGTGCACGAA |
| | CCCCCCGTTCAGCCCGACCGCTGCGCCTTATCCGGTAACTATCGTCTTGAGTCCAAC |
| | CCGGTAAGACACGACTTATCGCCACTGGCAGCAGCCACTGGTAACAGGATTAGCA |
| | GAGCGAGGTATGTAGGCGGTGCTACAGAGTTCTTGAAGTGGTGGCCTAACTACGG |
| | CTACACTAGAAGGACAGTATTTGGTATCTGCGCTCTGCTGAAGCCAGTTACCTTCG |
| | GAAAAAGAGTTGGTAGCTCTTGATCCGGCAAACAAACCACCGCTGGTAGCGGTGG |
| | TTTTTTTGTTTGCAAGCAGCAGATTACGCGCAGAAAAAAAGGATCTCAAGAAGATC |
| | CTTTGATCTTTTCTACGGGGTCTGACGCTCAGTGGAACGAAAACTCACGTTAAGGG |
| | ATTTTGGTCATGAGATTATCAAAAAGGATCTTCACCTAGATCCTTTTAAATTAAAAA |
| | TGAAGTTTTAAATCAATCTAAAGTATATATGAGTAAACTTGGTCTGACAGTTACCAA |
| | TGCTTAATCAGTGAGGCACCTATCTCAGCGATCTGTCTATTTCGTTCATCCATAGTT |
| | GCCTGACTCCCCGTCGTGTAGATAACTACGATACGGGAGGGCTTACCATCTGGCCC |
| | CAGTGCTGCAATGATACCGCGAGACCCACGCTCACCGGCTCCAGATTTATCAGCAA |
| | TAAACCAGCCAGCCGGAAGGGCCGAGCGCAGAAGTGGTCCTGCAACTTTATCCGC |
| | CTCCATCCAGTCTATTAATTGTTGCCGGGAAGCTAGAGTAAGTAGTTCGCCAGTTA |
| | ATAGTTTGCGCAACGTTGTTGCCATTGCTACAGGCATCGTGGTGTCACGCTCGTCG |
| | TTTGGTATGGCTTCATTCAGCTCCGGTTCCCAACGATCAAGGCGAGTTACATGATCC |
| | CCCATGTTGTGCAAAAAAGCGGTTAGCTCCTTCGGTCCTCCGATCGTTGTCAGAAG |
| | TAAGTTGGCCGCAGTGTTATCACTCATGGTTATGGCAGCACTGCATAATTCTCTTAC |
| | TGTCATGCCATCCGTAAGATGCTTTTCTGTGACTGGTGAGTACTCAACCAAGTCATT |
| | CTGAGAATAGTGTATGCGGCGACCGAGTTGCTCTTGCCCGGCGTCAATACGGGAT |
| | AATACCGCGCCACATAGCAGAACTTTAAAAGTGCTCATCATTGGAAAACGTTCTTC |
| | GGGGCGAAAACTCTCAAGGATCTTACCGCTGTTGAGATCCAGTTCGATGTAACCCA |
| | CTCGTGCACCCAACTGATCTTCAGCATCTTTTACTTTCACCAGCGTTTCTGGGTGAG |
| | CAAAAACAGGAAGGCAAAATGCCGCAAAAAAGGGAATAAGGGCGACACGGAAAT |
| | GTTGAATACTCATACTCTTCCTTTTTCAATATTATTGAAGCATTTATCAGGGTTATTG |
| | TCTCATGAGCGGATACATATTTGAATGTATTTAGAAAAATAAACAAATAGGGGTTC |
| | CGCGCACATTTCCCCGAAAA |
| 3 | pCI-mCD19-PDK1 retroviral vector DNA sequence |
| | GTGCCACCTGACGCGCCCTGTAGCGGCGCATTAAGCGCGGCGGGTGTGGTGGTTA |
| | CGCGCAGCGTGACCGCTACACTTGCCAGCGCCCTAGCGCCCGCTCCTTTCGCTTTCT |
| | TCCCTTCCTTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAAACTTGATT |
| | AGGGTGATGGTTCACGTAGTGGGCCATCGCCCTGATAGACGGTTTTTCGCCCTTTG |
| | ACGTTGGAGTCCACGTTCTTTAATAGTGGACTCTTGTTCCAAACTGGAACAACACTC |
| | AACCCTATCTCGGTCTATTCTTTTGATTTATAAGGGATTTTGCCGATTTCGGCCTATT |
| | GGTTAAAAAATGAGCTGATTTAACAAAAATTTAACGCGAATTTTAACAAAATATTA |
| | ACGCTTACAATTTCCATTCGCCATTCAGGCTGCGCAACTGTTGGGAAGGGCGATCG |
| | GTGCGGGCCTCTTCGCTATTACGCCAGCTGGCGAAAGGGGGATGTGCTGCAAGGC |
| | GATTAAGTTGGGTAACGCCAGGGTTTTCCCAGTCACGACGTTGTAAAACGACGGC |
| | CAGTGAGCGCGCGTAATACGACTCACTATAGGGCGAATTGGGTACCGGGCCCCCC |
| | CTCGACCGCGTTACATAACTTACGGTAAATGGCCCGCCTGGCTGACCGCCCAACGA |
| | CCCCCGCCCATTGACGTCAATAATGACGTATGTTCCCATAGTAACGCCAATAGGGA |
| | CTTTCCATTGACGTCAATGGGTGGAGTATTTACGGTAAACTGCCCACTTGGCAGTA |
| | CATCAAGTGTATCATATGCCAAGTACGCCCCCTATTGACGTCAATGACGGTAAATG |
| | GCCCGCCTGGCATTATGCCCAGTACATGACCTTATGGGACTTTCCTACTTGGCAGTA |
| | CATCTACGTATTAGTCATCGCTATTACCATGGTGATGCGGTTTTGGCAGTACATCAA |
| | TGGGCGTGGATAGCGGTTTGACTCACGGGGATTTCCAAGTCTCCACCCCATTGACG |
| | TCAATGGGAGTTTGTTTTGGCACCAAAATCAACGGGACTTTCCAAAATGTCGTAAC |
| | AACTCCGCCCCATTGACGCAAATGGGCGGTAGGCGTGTACGGTGGGAGGTCTATA |
| | TAAGCAGAGCTCGTTTAGTGAACCGgcGCGCCAGTCCTCCGATAGACTGCGTCGCC |
| | CGGGTACCCGTATTCCCAATAAAGCCTCTTGCTGTTTGCATCCGAATCGTGGACTCG |
| | CTGATCCTTGGGAGGGTCTCCTCAGATTGATTGACTGCCCACCTCGGGGGTCTTTC |
| | ATTTGGAGGTTCCACCGAGATTTGGAGACCCCTGCCCAGGGACCACCGACCCCCCC |
| | GCCGGGAGGTAAGCTGGCCAGCGGTCGTTTCGTGTCTGTCTCTGTCTTTGTGCGTG |
| | TTTGTGCCGGCATCTAATGTTTGCGCCTGCGTCTGTACTAGTTAGCTAACTAGCTCT |
| | GTATCTGGCGGACCCGTGGTGGAACTGACGAGTTCTGAACACCCGGCCGCAACCC |
| | TGGGAGACGTCCCAGGGACTTTGGGGGCCGTTTTTGTGGCCCGACCTGAGGAAGG |
| | GAGTCGATGTGGAATCCGACCCCGTCAGGATATGTGGTTCTGGTAGGAGACGAGA |
| | ACCTAAAACAGTTCCCGCCTCCGTCTGAATTTTTGCTTTCGGTTTGGAACCGAAGCC |
| | GCGCGTCTTGTCTGCTGCAGCGCTGCAGCATCGTTCTGTGTTGTCTCTGTCTGACTG |
| | TGTTTCTGTATTTGTCTGAAAATTAGGGCCAGACTGTTACCACTCCCTTAAGTTTGA |
| | CCTTAGGTCACTGGAAAGATGTCGAGCGGATCGCTCACAACCAGTCGGTAGATGT |
| | CAAGAAGAGACGTTGGGTTACCTTCTGCTCTGCAGAATGGCCAACCTTTAACGTCG |
| | GATGGCCGCGAGACGGCACCTTTAACCGAGACCTCATCACCCAGGTTAAGATCAA |
| | GGTCTTTTCACCTGGCCCGCATGGACACCCAGACCAGGTCCCCTACATCGTGACCT |
| | GGGAAGCCTTGGCTTTTGACCCCCCTCCCTGGGTCAAGCCCTTTGTACACCCTAAGC |
| | CTCCGCCTCCTCTTCCTCCATCCGCCCCGTCTCTCCCCCTTGAACCTCCTCGTTCGAC |
| | CCCGCCTCGATCCTCCCTTTATCCAGCCCTCACTCCTTCTCTAGGCGCCGgaattcacgc |
| | gtccgccaccATGAGGCTGGCAAGGCTGCTGCGGGGCGGCACGAGCGTCAGGCCGC |
| | TCTGCGCCGTCCCCTGCGCCAGCCGTAGCCTGGCCTCGGCCTCGGGTTCCGGGCCG |
| | GCGTCGGAGCTTGGCGTTCCGGGCCAGGTGGACTTCTATGCGCGCTTCTCGCCGTC |
| | GCCACTCTCCATGAAGCAGTTCCTGGACTTCGGGTCAGTGAATGCTTGTGAAAAGA |
| | CCTCGTTTATGTTTCTGCGACAAGAGTTGCCTGTTAGATTGGCAAATATAATGAAA |
| | GAAATCAGCCTTCTCCCCGATAATCTTCTCAGGACCCCATCCGTACAGCTGGTGCAA |
| | AGTTGGTATATCCAAAGCCTTCAGGAGTTGCTTGATTTTAAAGACAAAAGTGCTGA |
| | AGATGCTAAAACTATTTATGAATTCACAGACACAGTGATAAGGATCAGAAACCGGC |
| | ACAATGATGTCATTCCCACCATGGCCCAGGGTGTGACTGAATACAAGGAGAGCTTC |
| | GGGGTGGATCCTGTCACCAGCCAAAATGTTCAGTACTTTTTGGATCGATTCTACAT |
| | GAGTCGCATCTCAATTAGAATGCTACTCAACCAGCACTCCTTATTGTTCGGTGGAA |
| | AAGGAAGTCCATCTCATCGAAAGCACATTGGAAGCATAAATCCAAACTGCGACGT |
| | GGTGGAGGTCATTAAAGATGGCTATGAGAACGCTAGGCGGCTTTGTGATTTGTAT |
| | TATGTTAACTCTCCTGAACTTGAACTTGAAGAACTAAATGCGAAATCACCAGGACA |
| | GACAATACAAGTGGTTTATGTACCATCCCATCTCTATCACATGGTGTTTGAACTGTT |
| | CAAGAATGCCATGAGAGCGACCATGGAGCACCACGCGGACAAAGGCGTTTATCCC |
| | CCGATTCAGGTTCACGTCACGCTGGGCGAGGAGGATCTGACTGTGAAGATGAGTG |
| | ACCGGGGAGGCGGTGTTCCACTGAGGAAGATCGACAGACTCTTCAACTACATGTA |
| | CTCAACCGCACCCCGGCCTCGTGTTGAAACGTCCCGTGCTGTGCCCCTGGCTGGGT |
| | TTGGTTACGGATTGCCCATATCACGCCTCTATGCACAGTACTTCCAGGGAGACCTA |
| | AAGCTGTATTCCTTAGAGGGCTACGGGACAGATGCGGTTATCTACATTAAGGCTCT |
| | GTCGACAGAATCCGTCGAGAGACTCCCTGTGTACAATAAAGCCGCCTGGAAGCAT |
| | TACAAAGCCAACCACGAGGCTGACGACTGGTGTGTGCCCAGCCGAGAGCCGAAAG |
| | ATATGACCACATTCCGAAGCTCTTAAACCGGTCGACTACAAGGACGACGATGACAA |
| | GGGGCATCACCATCACCACCATTAGCTAGCGTTTAAACTTCCGCCCCTCTCCCTCCC |
| | CCCCCCCTAACGTTACTGGCCGAAGCCGCTTGGAATAAGGCCGGTGTGCGTTTGTC |
| | TATATGTTATTTTCCACCATATTGCCGTCTTTTGGCAATGTGAGGGCCCGGAAACCT |
| | GGCCCTGTCTTCTTGACGAGCATTCCTAGGGGTCTTTCCCCTCTCGCCAAAGGAATG |
| | CAAGGTCTGTTGAATGTCGTGAAGGAAGCAGTTCCTCTGGAAGCTTCTTGAAGACA |
| | AACAACGTCTGTAGCGACCCTTTGCAGGCAGCGGAACCCCCCACCTGGCGACAGG |
| | TGCCTCTGCGGCCAAAAGCCACGTGTATAAGATACACCTGCAAAGGCGGCACAAC |
| | CCCAGTGCCACGTTGTGAGTTGGATAGTTGTGGAAAGAGTCAAATGGCTCTCCTCA |
| | AGCGTATTCAACAAGGGGCTGAAGGATGCCCAGAAGGTACCCCATTGTATGGGAT |
| | CTGATCTGGGGCCTCGGTACACATGCTTTACATGTGTTTAGTCGAGGTTAAAAAAA |
| | CGTCTAGGCCCCCCGAACCACGGGGACGTGGTTTTCCTTTGAAAAACACGATGATA |
| | ATATGGCCACCATGCCCTCCCCTCTGCCCGTGAGCTTCCTGCTGTTCCTGACCCTGG |
| | TGGGAGGCAGGCCCCAGAAGAGCCTGCTGGTGGAGGTGGAGGAAGGCGGCAAC |
| | GTGGTGCTGCCCTGTCTGCCTGATAGCTCCCCTGTGAGCAGCGAGAAGCTGGCCTG |
| | GTACAGGGGCAACCAGAGCACCCCTTTCCTGGAACTGAGCCCTGGAAGCCCTGGA |
| | CTGGGCCTGCACGTGGGAAGCCTGGGCATCCTGCTGGTGATCGTGAACGTGTCCG |
| | ACCACATGGGCGGCTTCTATCTGTGCCAGAAGAGGCCCCCCTTCAAAGACATCTGG |
| | CAGCCCGCCTGGACCGTGAACGTGGAGGACAGCGGCGAGATGTTCAGGTGGAAC |
| | GCCAGCGACGTCAGGGACCTGGACTGCGACCTGAGAAACAGAAGCTCCGGCTCCC |
| | ACAGGAGCACAAGCGGCAGCCAGCTGTACGTGTGGGCTAAGGACCACCCTAAGG |
| | TGTGGGGCACAAAGCCCGTGTGTGCCCCTAGGGGAAGCTCCCTCAACCAGAGCCT |
| | GATCAACCAGGACCTGACAGTGGCCCCCGGAAGCACCCTGTGGCTGAGCTGTGGC |
| | GTGCCTCCTGTGCCTGTGGCCAAAGGATCCATCTCCTGGACCCACGTGCACCCCAG |
| | AAGGCCCAACGTGAGCCTGCTGAGCCTGAGCCTGGGAGGCGAACACCCCGTGAG |
| | GGAGATGTGGGTGTGGGGCAGCCTGCTGCTGCTGCCTCAGGCTACCGCCCTGGAC |
| | GAAGGCACCTACTACTGTCTGAGGGGCAACCTGACAATCGAGAGGCACGTGAAG |
| | GTGATCGCTAGAAGCGCCGTGTGGCTGTGGCTGCTGAGGACCGGCGGATGGATT |
| | GTGCCCGTGGTGACCCTGGTGTACGTGATCTTCTGCATGGTGTCCCTGGTGGCCTT |
| | CCTGTACTGTCAGAGAGCCTTCATCCTCAGGAGAAAGAGGAAGAGGATGACATAG |
| | TGAttaacgcggccAATTCGATAAAATAAAAGATTTTATTTAGTCTCCAGAAAAAGGG |
| | GGGAATGAAAGACCCCACCTGTAGGTTTGGCAAGCTAGCTTAAGTAACGCCATTTT |
| | GCAAGGCATGGAAAATACATAACTGAGAATAGAGAAGTTCAGATCAAGGTTAGGA |
| | ACAGAGAGACAGCAGAATATGGGCCAAACAGGATATCTGTGGTAAGCAGTTCCTG |
| | CCCCGGCTCAGGGCCAAGAACAGATGGTCCCCAGATGCGGTCCCGCCCTCAGCAG |
| | TTTCTAGAGAACCATCAGATGTTTCCAGGGTGCCCCAAGGACCTGAAATGACCCTG |
| | TGCCTTATTTGAACTAACCAATCAGTTCGCTTCTCGCTTCTGTTCGCGCGCTTCTGCT |
| | CCCCGAGCTCAATAAAAGAGCCCACAACCCCTCACTCGGCGCGCCAGTCCTCCGAT |
| | AGACTGCGTCGCCCGGGTACCCGTGTATCCAATAAACCCTCTTGCAGTTGCATCCG |
| | ACTTGTGGTCTCGCTGTTCCTTGGGAGGGTCTCCTCTGAGTGATTGACTACCCGTCA |
| | GCGGGGGTCTTTCAGCGGCCGGCCGCCACCGCGGTGGAGCTCCAGCTTTTGTTCC |
| | CTTTAGTGAGGGTTAATTGCGCGCTTGGCGTAATCATGGTCATAGCTGTTTCCTGT |
| | GTGAAATTGTTATCCGCTCACAATTCCACACAACATACGAGCCGGAAGCATAAAGT |
| | GTAAAGCCTGGGGTGCCTAATGAGTGAGCTAACTCACATTAATTGCGTTGCGCTCA |
| | CTGCCCGCTTTCCAGTCGGGAAACCTGTCGTGCCAGCTGCATTAATGAATCGGCCA |
| | ACGCGCGGGGAGAGGCGGTTTGCGTATTGGGCGCTCTTCCGCTTCCTCGCTCACTG |
| | ACTCGCTGCGCTCGGTCGTTCGGCTGCGGCGAGCGGTATCAGCTCACTCAAAGGC |
| | GGTAATACGGTTATCCACAGAATCAGGGGATAACGCAGGAAAGAACATGTGAGCA |
| | AAAGGCCAGCAAAAGGCCAGGAACCGTAAAAAGGCCGCGTTGCTGGCGTTTTTCC |
| | ATAGGCTCCGCCCCCCTGACGAGCATCACAAAAATCGACGCTCAAGTCAGAGGTG |
| | GCGAAACCCGACAGGACTATAAAGATACCAGGCGTTTCCCCCTGGAAGCTCCCTCG |
| | TGCGCTCTCCTGTTCCGACCCTGCCGCTTACCGGATACCTGTCCGCCTTTCTCCCTTC |
| | GGGAAGCGTGGCGCTTTCTCATAGCTCACGCTGTAGGTATCTCAGTTCGGTGTAGG |
| | TCGTTCGCTCCAAGCTGGGCTGTGTGCACGAACCCCCCGTTCAGCCCGACCGCTGC |
| | GCCTTATCCGGTAACTATCGTCTTGAGTCCAACCCGGTAAGACACGACTTATCGCC |
| | ACTGGCAGCAGCCACTGGTAACAGGATTAGCAGAGCGAGGTATGTAGGCGGTGC |
| | TACAGAGTTCTTGAAGTGGTGGCCTAACTACGGCTACACTAGAAGGACAGTATTTG |
| | GTATCTGCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCAGCAGAT |
| | TACGCGCAGAAAAAAAGGATCTCAAGAAGATCCTTTGATCTTTTCTACGGGGTCTG |
| | ACGCTCAGTGGAACGAAAACTCACGTTAAGGGATTTTGGTCATGAGATTATCAAAA |
| | AGGATCTTCACCTAGATCCTTTTAAATTAAAAATGAAGTTTTAAATCAATCTAAAGT |
| | ATATATGAGTAAACTTGGTCTGACAGTTACCAATGCTTAATCAGTGAGGCACCTAT |
| | CTCAGCGATCTGTCTATTTCGTTCATCCATAGTTGCCTGACTCCCCGTCGTGTAGAT |
| | AACTACGATACGGGAGGGCTTACCATCTGGCCCCAGTGCTGCAATGATACCGCGA |
| | GACCCACGCTCACCGGCTCCAGATTTATCAGCAATAAACCAGCCAGCCGGAAGGG |
| | CCGAGCGCAGAAGTGGTCCTGCAACTTTATCCGCCTCCATCCAGTCTATTAATTGTT |
| | GCCGGGAAGCTAGAGTAAGTAGTTCGCCAGTTAATAGTTTGCGCAACGTTGTTGCC |
| | ATTGCTACAGGCATCGTGGTGTCACGCTCGTCGTTTGGTATGGCTTCATTCAGCTCC |
| | GGTTCCCAACGATCAAGGCGAGTTACATGATCCCCCATGTTGTGCAAAAAAGCGGT |
| | TAGCTCCTTCGGTCCTCCGATCGTTGTCAGAAGTAAGTTGGCCGCAGTGTTATCACT |
| | CATGGTTATGGCAGCACTGCATAATTCTCTTACTGTCATGCCATCCGTAAGATGCTT |
| | TTCTGTGACTGGTGAGTACTCAACCAAGTCATTCTGAGAATAGTGTATGCGGCGAC |
| | CGAGTTGCTCTTGCCCGGCGTCAATACGGGATAATACCGCGCCACATAGCAGAACT |
| | TTAAAAGTGCTCATCATTGGAAAACGTTCTTCGGGGCGAAAACTCTCAAGGATCTT |
| | ACCGCTGTTGAGATCCAGTTCGATGTAACCCACTCGTGCACCCAACTGATCTTCAGC |
| | ATCTTTTACTTTCACCAGCGTTTCTGGGTGAGCAAAAACAGGAAGGCAAAATGCCG |
| | CAAAAAAGGGAATAAGGGCGACACGGAAATGTTGAATACTCATACTCTTCCTTTTT |
| | CAATATTATTGAAGCATTTATCAGGGTTATTGTCTCATGAGCGGATACATATTTGAA |
| | TGTATTTAGAAAAATAAACAAATAGGGGTTCCGCGCACATTTCCCCGAAAA |
| 4 | Mouse PDP1 protein coding sequence |
| | ATGGAGCGGCGGCGGTGCGCCTGTCCGGGCTCCTGGAGTTGCCCGCCGGGGGCT |
| | GTGATGCCGCCTCGCCTGCCGGGAATCCCAGTCAGAACGTCCAGTCTGCCACTGTT |
| | CTCTGATGCCATGCCAGCACCAACTCAACTGTTTTTTCCTCTCGTCCGTAACTGTGA |
| | ACTGAGCAGAATCTATGGCACTGCATGTTACTGCCACCACAAACATCTCTGCTGTTC |
| | ACCACCATACATTCCTCAGAATCGTCTGAGGTACACACCCCATCCTGCATATGCTAC |
| | CTTTTGTAGGCCACGGGAGAACTGGTGGCAGTATACTCAAGGAAGGAGATACGCT |
| | TCTACACCGCAGAAATTTTACCTCACACCTCCACAAGTCAACAGCATCCTTAAAGCT |
| | AATGAATACAGCTTCAAAGTACCAGAATTTGATGGCAAAAATGTCAGTTCCATTCT |
| | TGGATTTGACAGCAATCAGCTGCCTGCAAATGCACCCATAGAGGACCGGAGAAGT |
| | GCAGCAACCTGCTTGCAGACCAGAGGGATGCTCTTGGGGGTTTTTGATGGTCATG |
| | CAGGCTGTGCTTGTTCCCAGGCAGTCAGTGAAAGACTCTTCTATTATATTGCTGTTT |
| | CCTTGTTGCCCCATGAGACTTTGCTAGAGATTGAAAATGCAGTGGAGAGTGGTCG |
| | GGCACTGCTACCTATCCTTCAGTGGCACAAGCACCCCAATGATTACTTCAGTAAGG |
| | AGGCGTCCAAATTGTATTTCAACAGCTTGAGGACTTACTGGCAAGAACTCATAGAC |
| | CTCAATACTGGAGAATCAGCTGATATTGATGTTAAGGAGGCTTTAATTAATGCTTTC |
| | AAGAGACTCGATAATGACATTTCATTGGAGGCTCAAGTTGGTGATCCTAATTCTTTT |
| | CTAAATTACCTGGTGCTTCGGGTAGCATTTTCTGGGGCTACTGCTTGTGTGGCCCAT |
| | GTAGATGGTGTTGACCTCCATGTGGCTAACACTGGTGATAGTAGAGCCATGCTAG |
| | GTGTGCAAGAAGAAGATGGCTCTTGGTCAGCAGTCACACTCTCTAATGACCACAAT |
| | GCTCAGAATGAAAGAGAACTAGAGCGTCTGAAACTGGAACACCCAAAAAATGAG |
| | GCCAAGAGCGTGGTAAAGCAGGACCGGCTGCTTGGCTTGCTGATGCCCTTTAGGG |
| | CTTTTGGAGATGTAAAGTTCAAATGGAGCATTGACCTTCAAAAGAGAGTGATAGA |
| | GTCTGGCCCAGACCAGTTGAATGACAATGAATACACCAAGTTTATCCCTCCTAACT |
| | ATCATACACCTCCTTATCTTACTGCTGAGCCAGAGGTAACTTATCACAGATTAAGGC |
| | CACAGGATAAATTCCTAGTGTTAGCAACTGATGGGTTGTGGGAGACTATGCATAG |
| | ACAGGATGTGGTTAGGATTGTGGGTGAATACTTAACTGGTATGCATCACCAACAG |
| | CCAATAGCAGTTGGTGGGTACAAGGTGACTCTGGGACAGATGCATGGCCTTTTAA |
| | CAGAAAGGAGAGCAAAGATGTCATCAGTGTTTGAGGATCAGAATGCAGCAACCCA |
| | TCTCATTCGCCATGCTGTAGGCAATAATGAATTTGGGGCTGTTGATCATGAACGAC |
| | TCTCTAAAATGCTTAGTCTTCCTGAAGAGCTTGCTCGGATGTATAGAGATGACATTA |
| | CAATCATTGTAGTTCAGTTCAATTCTCATGTTGTAGGGGCATACCAAAACCAGGAA |
| | CAGTAA |
| 5 | Mouse PDK1 protein coding sequence |
| | ATGAGGCTGGCAAGGCTGCTGCGGGGCGGCACGAGCGTCAGGCCGCTCTGCGCC |
| | GTCCCCTGCGCCAGCCGTAGCCTGGCCTCGGCCTCGGGTTCCGGGCCGGCGTCGG |
| | AGCTTGGCGTTCCGGGCCAGGTGGACTTCTATGCGCGCTTCTCGCCGTCGCCACTC |
| | TCCATGAAGCAGTTCCTGGACTTCGGGTCAGTGAATGCTTGTGAAAAGACCTCGTT |
| | TATGTTTCTGCGACAAGAGTTGCCTGTTAGATTGGCAAATATAATGAAAGAAATCA |
| | GCCTTCTCCCCGATAATCTTCTCAGGACCCCATCCGTACAGCTGGTGCAAAGTTGGT |
| | ATATCCAAAGCCTTCAGGAGTTGCTTGATTTTAAAGACAAAAGTGCTGAAGATGCT |
| | AAAACTATTTATGAATTCACAGACACAGTGATAAGGATCAGAAACCGGCACAATG |
| | ATGTCATTCCCACCATGGCCCAGGGTGTGACTGAATACAAGGAGAGCTTCGGGGT |
| | GGATCCTGTCACCAGCCAAAATGTTCAGTACTTTTTGGATCGATTCTACATGAGTCG |
| | CATCTCAATTAGAATGCTACTCAACCAGCACTCCTTATTGTTCGGTGGAAAAGGAA |
| | GTCCATCTCATCGAAAGCACATTGGAAGCATAAATCCAAACTGCGACGTGGTGGA |
| | GGTCATTAAAGATGGCTATGAGAACGCTAGGCGGCTTTGTGATTTGTATTATGTTA |
| | ACTCTCCTGAACTTGAACTTGAAGAACTAAATGCGAAATCACCAGGACAGACAATA |
| | CAAGTGGTTTATGTACCATCCCATCTCTATCACATGGTGTTTGAACTGTTCAAGAAT |
| | GCCATGAGAGCGACCATGGAGCACCACGCGGACAAAGGCGTTTATCCCCCGATTC |
| | AGGTTCACGTCACGCTGGGCGAGGAGGATCTGACTGTGAAGATGAGTGACCGGG |
| | GAGGCGGTGTTCCACTGAGGAAGATCGACAGACTCTTCAACTACATGTACTCAACC |
| | GCACCCCGGCCTCGTGTTGAAACGTCCCGTGCTGTGCCCCTGGCTGGGTTTGGTTA |
| | CGGATTGCCCATATCACGCCTCTATGCACAGTACTTCCAGGGAGACCTAAAGCTGT |
| | ATTCCTTAGAGGGCTACGGGACAGATGCGGTTATCTACATTAAGGCTCTGTCGACA |
| | GAATCCGTCGAGAGACTCCCTGTGTACAATAAAGCCGCCTGGAAGCATTACAAAG |
| | CCAACCACGAGGCTGACGACTGGTGTGTGCCCAGCCGAGAGCCGAAAGATATGAC |
| | CACATTCCGAAGCTCTTAA |
| 6 | Human PDP1 protein coding sequence |
| | ATGCCAGCACCAACTCAACTGTTTTTTCCTCTCATCCGTAACTGTGAACTGAGCAGG |
| | ATCTATGGCACTGCATGTTACTGCCACCACAAACATCTCTGTTGTTCCTCATCGTAC |
| | ATTCCTCAGAGTCGACTGAGATACACACCTCATCCAGCATATGCTACCTTTTGCAGG |
| | CCAAAGGAGAACTGGTGGCAGTACACCCAAGGAAGGAGATATGCTTCCACACCAC |
| | AGAAATTTTACCTCACACCTCCACAAGTCAATAGCATCCTTAAAGCTAATGAATACA |
| | GTTTCAAAGTGCCAGAATTTGACGGCAAAAATGTCAGTTCTATCCTTGGATTTGAC |
| | AGCAATCAGCTGCCTGCAAATGCACCCATTGAGGACCGGAGAAGTGCAGCAACCT |
| | GCTTGCAGACCAGAGGGATGCTTTTGGGGGTTTTTGATGGCCATGCAGGTTGTGC |
| | TTGTTCCCAGGCAGTCAGTGAAAGACTCTTTTATTATATTGCTGTCTCTTTGTTACCC |
| | CATGAGACTTTGCTAGAGATTGAAAATGCAGTGGAGAGCGGCCGGGCACTGCTAC |
| | CCATTCTCCAGTGGCACAAGCACCCCAATGATTACTTTAGTAAGGAGGCATCCAAA |
| | TTGTACTTTAACAGCTTGAGGACTTACTGGCAAGAGCTTATAGACCTCAACACTGG |
| | TGAGTCGACTGATATTGATGTTAAGGAGGCTCTAATTAATGCCTTCAAGAGGCTTG |
| | ATAATGACATCTCCTTGGAGGCGCAAGTTGGTGATCCTAATTCTTTTCTCAACTACC |
| | TGGTGCTTCGAGTGGCATTTTCTGGAGCCACTGCTTGTGTGGCCCATGTGGATGGT |
| | GTTGACCTTCATGTGGCCAATACTGGCGATAGCAGAGCCATGCTGGGTGTGCAGG |
| | AAGAGGACGGCTCATGGTCAGCAGTCACGCTGTCTAATGACCACAATGCTCAAAA |
| | TGAAAGAGAACTAGAACGGCTGAAATTGGAACATCCAAAGAGTGAGGCCAAGAG |
| | TGTCGTGAAACAGGATCGGCTGCTTGGCTTGCTGATGCCATTTAGGGCATTTGGAG |
| | ATGTAAAGTTCAAATGGAGCATTGACCTTCAAAAGAGAGTGATAGAATCTGGCCC |
| | AGACCAGTTGAATGACAATGAATATACCAAGTTTATTCCTCCTAATTATCACACACC |
| | TCCTTATCTCACTGCTGAGCCAGAGGTAACTTACCACCGATTAAGGCCACAGGATA |
| | AGTTTCTGGTGTTGGCTACTGATGGGTTGTGGGAGACTATGCATAGGCAGGATGT |
| | GGTTAGGATTGTGGGTGAGTACCTAACTGGCATGCATCACCAACAGCCAATAGCT |
| | GTTGGTGGCTACAAGGTGACTCTGGGACAGATGCATGGCCTTTTAACAGAAAGGA |
| | GAACCAAAATGTCCTCGGTATTTGAGGATCAGAACGCAGCAACCCATCTCATTCGC |
| | CACGCTGTGGGCAACAACGAGTTTGGGACTGTTGATCATGAGCGCCTCTCTAAAAT |
| | GCTTAGTCTTCCTGAAGAGCTTGCTCGAATGTACAGAGATGACATTACAATCATTG |
| | TAGTTCAGTTCAATTCTCATGTTGTAGGGGCGTATCAAAACCAAGAATAG |
| 7 | Human PDK1 protein coding sequence |
| | ATGAGGCTGGCGCGGCTGCTTCGCGGAGCCGCCTTGGCCGGCCCGGGCCCGGGG |
| | CTGCGCGCCGCCGGCTTCAGCCGCAGCTTCAGCTCGGACTCGGGCTCCAGCCCGG |
| | CGTCCGAGCGCGGCGTTCCGGGCCAGGTGGACTTCTACGCGCGCTTCTCGCCGTCC |
| | CCGCTCTCCATGAAGCAGTTCCTGGACTTCGGATCAGTGAATGCTTGTGAAAAGAC |
| | CTCATTTATGTTTCTGCGGCAAGAGTTGCCTGTCAGACTGGCAAATATAATGAAAG |
| | AAATAAGTCTCCTTCCAGATAATCTTCTCAGGACACCATCCGTTCAATTGGTACAAA |
| | GCTGGTATATCCAGAGTCTTCAGGAGCTTCTTGATTTTAAGGACAAAAGTGCTGAG |
| | GATGCTAAAGCTATTTATGACTTTACAGATACTGTGATACGGATCAGAAACCGACA |
| | CAATGATGTCATTCCCACAATGGCCCAGGGTGTGATTGAATACAAGGAGAGCTTTG |
| | GGGTGGATCCTGTCACCAGCCAGAATGTTCAGTACTTTTTGGATCGATTCTACATG |
| | AGTCGCATTTCAATTAGAATGTTACTCAATCAGCACTCTTTATTGTTTGGTGGAAAA |
| | GGCAAAGGAAGTCCATCTCATCGAAAACACATTGGAAGCATAAATCCAAACTGCA |
| | ATGTACTTGAAGTTATTAAAGATGGCTATGAAAATGCTAGGCGTCTGTGTGATTTG |
| | TATTATATTAACTCTCCCGAACTAGAACTTGAAGAACTAAATGCAAAATCACCAGG |
| | ACAGCCAATACAAGTGGTTTATGTACCATCCCATCTCTATCACATGGTGTTTGAACT |
| | TTTCAAGAATGCAATGAGAGCCACTATGGAACACCATGCCAACAGAGGTGTTTACC |
| | CCCCTATTCAAGTTCATGTCACGCTGGGTAATGAGGATTTGACTGTGAAGATGAGT |
| | GACCGAGGAGGTGGCGTTCCTTTGAGGAAAATTGACAGACTTTTCAACTACATGTA |
| | TTCAACTGCACCAAGACCTCGTGTTGAGACCTCCCGCGCAGTGCCTCTGGCTGGTT |
| | TTGGTTATGGATTGCCCATATCACGTCTTTACGCACAATACTTCCAAGGAGACCTGA |
| | AGCTGTATTCCCTAGAGGGTTACGGGACAGATGCAGTTATCTACATTAAGGCTCTG |
| | TCAACAGACTCAATAGAAAGACTCCCAGTGTATAACAAAGCTGCCTGGAAGCATTA |
| | CAACACCAACCACGAGGCTGATGACTGGTGCGTCCCCAGCAGAGAACCCAAAGAC |
| | ATGACGACGTTCCGCAGTGCCTAG |
| 8 | Mouse PDP1 amino acid sequence |
| | MERRRCACPGSWSCPPGAVMPPRLPGIPVRTSSLPLFSDAMPAPTQLFFPLVRNCELS |
| | RIYGTACYCHHKHLCCSPPYIPQNRLRYTPHPAYATFCRPRENWWQYTQGRRYASTP |
| | QKFYLTPPQVNSILKANEYSFKVPEFDGKNVSSILGFDSNQLPANAPIEDRRSAATCLQT |
| | RGMLLGVFDGHAGCACSQAVSERLFYYIAVSLLPHETLLEIENAVESGRALLPILQWHK |
| | HPNDYFSKEASKLYFNSLRTYWQELIDLNTGESADIDVKEALINAFKRLDNDISLEAQVG |
| | DPNSFLNYLVLRVAFSGATACVAHVDGVDLHVANTGDSRAMLGVQEEDGSWSAVTL |
| | SNDHNAQNERELERLKLEHPKNEAKSVVKQDRLLGLLMPFRAFGDVKFKWSIDLQKR |
| | VIESGPDQLNDNEYTKFIPPNYHTPPYLTAEPEVTYHRLRPQDKFLVLATDGLWETMH |
| | RQDVVRIVGEYLTGMHHQQPIAVGGYKVTLGQMHGLLTERRAKMSSVFEDQNAAT |
| | HLIRHAVGNNEFGAVDHERLSKMLSLPEELARMYRDDITIIVVQFNSHVVGAYQNQEQ |
| 9 | Mouse PDK1 amino acid sequence |
| | MRLARLLRGGTSVRPLCAVPCASRSLASASGSGPASELGVPGQVDFYARFSPSPLSMK |
| | QFLDFGSVNACEKTSFMFLRQELPVRLANIMKEISLLPDNLLRTPSVQLVQSWYIQSLQ |
| | ELLDFKDKSAEDAKTIYEFTDTVIRIRNRHNDVIPTMAQGVTEYKESFGVDPVTSQNVQ |
| | YFLDRFYMSRISIRMLLNQHSLLFGGKGSPSHRKHIGSINPNCDVVEVIKDGYENARRLC |
| | DLYYVNSPELELEELNAKSPGQTIQVVYVPSHLYHMVFELFKNAMRATMEHHADKGV |
| | YPPIQVHVTLGEEDLTVKMSDRGGGVPLRKIDRLFNYMYSTAPRPRVETSRAVPLAGF |
| | GYGLPISRLYAQYFQGDLKLYSLEGYGTDAVIYIKALSTESVERLPVYNKAAWKHYKANH |
| | EADDWCVPSREPKDMTTFRSS |
| 10 | Human PDP1 amino acid sequence |
| | MPAPTQLFFPLIRNCELSRIYGTACYCHHKHLCCSSSYIPQSRLRYTPHPAYATFCRPKEN |
| | WWQYTQGRRYASTPQKFYLTPPQVNSILKANEYSFKVPEFDGKNVSSILGFDSNQLPA |
| | NAPIEDRRSAATCLQTRGMLLGVFDGHAGCACSQAVSERLFYYIAVSLLPHETLLEIENA |
| | VESGRALLPILQWHKHPNDYFSKEASKLYFNSLRTYWQELIDLNTGESTDIDVKEALINA |
| | FKRLDNDISLEAQVGDPNSFLNYLVLRVAFSGATACVAHVDGVDLHVANTGDSRAML |
| | GVQEEDGSWSAVTLSNDHNAQNERELERLKLEHPKSEAKSVVKQDRLLGLLMPFRAF |
| | GDVKFKWSIDLQKRVIESGPDQLNDNEYTKFIPPNYHTPPYLTAEPEVTYHRLRPQDKF |
| | LVLATDGLWETMHRQDVVRIVGEYLTGMHHQQPIAVGGYKVTLGQMHGLLTERRTK |
| | MSSVFEDQNAATHLIRHAVGNNEFGTVDHERLSKMLSLPEELARMYRDDITIIVVQFN |
| | SHVVGAYQNQE |
| 11 | Human PDK1 amino acid sequence |
| | MRLARLLRGAALAGPGPGLRAAGFSRSFSSDSGSSPASERGVPGQVDFYARFSPSPLS |
| | MKQFLDFGSVNACEKTSFMFLRQELPVRLANIMKEISLLPDNLLRTPSVQLVQSWYIQS |
| | LQELLDFKDKSAEDAKAIYDFTDTVIRIRNRHNDVIPTMAQGVIEYKESFGVDPVTSQN |
| | VQYFLDRFYMSRISIRMLLNQHSLLFGGKGKGSPSHRKHIGSINPNCNVLEVIKDGYEN |
| | ARRLCDLYYINSPELELEELNAKSPGQPIQVVYVPSHLYHMVFELFKNAMRATMEHHA |
| | NRGVYPPIQVHVTLGNEDLTVKMSDRGGGVPLRKIDRLFNYMYSTAPRPRVETSRAVP |
| | LAGFGYGLPISRLYAQYFQGDLKLYSLEGYGTDAVIYIKALSTDSIERLPVYNKAAWKHY |
| | NTNHEADDWCVPSREPKDMTTFRSA |

We claim:

1. Isolated or recombinant T cells, optionally human primary T cells, further optionally human primary CD4+ or CD8+ T cells, which are engineered to stably and/or heritably express increased amounts of pyruvate dehydrogenase kinase enzyme (PDK) and/or pyruvate dehydrogenase phosphatase (PDP) enzyme, or progeny thereof.

2. Isolated or recombinant T cells, optionally human primary T cells according to claim 1, wherein
(i) the expression of a gene encoding said pyruvate dehydrogenase kinase enzyme (PDK) and/or pyruvate dehydrogenase phosphatase (PDP) enzyme is virally mediated;
(ii) the expression of a gene encoding said pyruvate dehydrogenase kinase enzyme (PDK) and/or pyruvate dehydrogenase phosphatase (PDP) enzyme is mediated by a retroviral, lentiviral, adenoviral, adeno-associated viral or herpes simplex viral vector sequence and/or by a gene editing technique;
(iii) the expression of a gene encoding said pyruvate dehydrogenase kinase enzyme (PDK) and/or pyruvate dehydrogenase phosphatase (PDP) enzyme is retrovirally mediated;
(iv) the gene encoding said pyruvate dehydrogenase kinase enzyme (PDK) and/or pyruvate dehydrogenase is introduced by use of a gene editing technique, optionally wherein said gene editing method comprises the use of TALEN (transcription activator-like effector nuclease) mediated gene editing, CRSPR (Clustered Regularly Interspaced Short Palindromic Repeats) mediated gene editing, double strand repair, meganuclease mediated gene editing, zinc finger nuclease mediated gene editing, Nucleobase Modification (BASE Editing) or ARCUT (artificial restriction DNA cutter) mediated gene editing;
(v) the cells are expanded and/or permitted to proliferate in vitro;
(vi) the cells comprise multiple copies of gene encoding said pyruvate dehydrogenase kinase enzyme (PDK) and/or pyruvate dehydrogenase phosphatase (PDP) enzyme;
(vii) the expression of a gene encoding said pyruvate dehydrogenase kinase enzyme (PDK) and/or pyruvate dehydrogenase phosphatase (PDP) enzyme is controlled by a constitutive promoter;
(viii) the expression of a gene encoding said pyruvate dehydrogenase kinase enzyme (PDK) and/or pyruvate dehydrogenase phosphatase (PDP) enzyme is controlled by an inducible promoter;
(ix) the expression of a gene encoding said pyruvate dehydrogenase kinase enzyme (PDK) and/or pyruvate dehydrogenase phosphatase (PDP) enzyme is controlled by a tissue-specific promoter;
(x) the expression of a gene encoding said pyruvate dehydrogenase kinase enzyme (PDK) and/or pyruvate dehydrogenase phosphatase (PDP) enzyme is controlled by a promoter selected from cytomegalovirus enhancer/chicken 3-actin (CAG) and elongation factor (EF)-1α promoters;
(xi) the cells further comprise an inducible suicide gene;
(xii) the cells are derived from one or more of: peripheral T cells, bone marrow T cells, Tumor Infiltrating Lymphocytes, autologous T cells, allogeneic T cells, hematopoietic stem cells, and/or induced pluripotent stem cells;
(xiii) the T cells comprise CD4+ Tcells and/or CD8+ Tcells, optionally primary human CD4+ T cells and/or CD8+ T cells or progeny thereof;
(xiv) the expressed Pdp1 comprises an amino acid sequence possessing at least 80, 85, 90, 91, 92, 93, 94, 95, 96, 97, 98, 99 or 100% sequence identity to the human Pdp1 of SEQ ID NO: 10 or the murine Pdp1 of SEQ ID NO: 8;
(xv) the expressed Pdp1 polypeptide comprises an amino acid sequence possessing at least 80, 85, 90, 91, 92, 93, 94, 95, 96, 97, 98, 99 or 100% sequence identity to the human Pdk1 of SEQ ID NO: 11 or the murine Pdk1 of SEQ ID NO: 9;
(xvi) the nucleic acid sequence encoding the expressed Pdp1 polypeptide comprises a nucleic acid sequence possessing at least 80, 85, 90, 91, 92, 93, 94, 95, 96, 97, 98, 99 or 100% sequence identity to the human Pdk1 nucleic acid sequence of SEQ ID NO: 7 or the murine Pdk1 nucleic acid sequence of SEQ ID NO: 5;
(xvii) the nucleic acid encoding the expressed Pdp1 polypeptide comprises a nucleic acid sequence possessing at least 80, 85, 90, 91, 92, 93, 94, 95, 96, 97, 98, 99 or 100% sequence identity to the human Pdp1 nucleic acid sequence of SEQ ID NO: 6 or the murine Pdp1 nucleic acid sequence of SEQ ID NO: 4; or
(xviii) any combination of (i) to (xvii).

3. Isolated or recombinant T cells according to claim 1 or 2, wherein:
(a) said T cells are more metabolically active, or
(b) said T cells have an increased rate of glycolysis, or
(c) said T cells have an increased rate of oxidative phosphorylation, or
(d) said T cells exhibit more active effector functions comprising production of Granzyme B, TNFα, and/or Interferon γ; or
(e) any combination of (a) to (d).

4. Isolated or recombinant T cells according to any one of the foregoing claims which are further engineered to comprise a desired targeting or therapeutic moiety, optionally wherein the desired targeting or therapeutic moiety comprises:
(a) a chimeric antigen receptor, or
(b) an exogenous T cell receptor, or
(c) a cytokine, or
(d) a cytokine receptor, or
(e) a chemokine, or
(f) a chemokine receptor,
(g) an immune checkpoint modulator, or
(h) any combination of (a) to (g).

5. The isolated or recombinant T cells of claim 4 wherein
(i) the chimeric antigen receptor(s) or exogenous TCR(s) targets an antigen expressed by a tumor, site of infection, site of inflammation, and/or a site of autoimmunity in a treated subject;
(ii) the chimeric antigen receptor(s) or exogenous TCR(s) targets one or more of alphafetoprotein, BCMA, B7-H3, Carcinoembryonic antigen, CA-135, CD19, CD133, Claudin 18.2, c-Met, EGFR, FAP, GD2, GPC3, HER-2, MAGE, Mesothelin, MUC-1, MUC-16, NY-ESO-1, PD-L1, PSCA, PSMA, ROR1, or Tyrosinase;
(iii) the immune checkpoint modulator comprises an antagonist antibody to one or more of: PD-1, PD-L1, PD-L2, CTLA-4, TIM-3, LAG-3, CD47, SIRPα, B7-H3; or
(iv) any combination of (i) to (iii).

6. A composition comprising isolated or recombinant T cells or progeny of any one of the foregoing claims which optionally:
(i) is lyophilized;
(ii) further comprises a therapeutic agent, optionally a cytokine, cytokine receptor, chemokine, chemokine receptor, immune checkpoint inhibitor agonist or antagonist;
(iii) further comprises a hormone, cytokine, cytokine antagonist, therapeutic antibody, immunostimulatory antibody or fusion protein, immunoinhibitory antibody or fusion protein, anti-inflammatory agent, checkpoint inhibitor or checkpoint inhibitor agonist or antagonist, e.g., a polypeptide which promotes or reduces the expression or activity of a stimulatory immune checkpoint molecule such as CD27, CD28, CD40, CD122, CD137, OX40, GITR or ICOS and/or a polypeptide which promotes or reduces the expression or activity of an inhibitory immune checkpoint molecule such as A2AR, A2BR, B7-H3, B7-H4, BTLA, CTLA-4, IDO, KIR, LAG3, NOX2, PD-1, PD-L1, VISTA or SIGLEC7);
(iv) further comprises and/or the engineered T cells according to any of the prior claims are further engineered to comprise and express a gene encoding one of IL-2, IL-4, IL-5, IL-7, IL-10, IL-12p40, IL-12p70, IL-15, and interferon (IFN) gamma;
(v) further comprises and/or the engineered T cells according to any of the prior claims are further engineered to comprise and express a gene encoding one of a pro-inflammatory cytokine, e.g., IL-1a, IL-1b, IL-6, IL-13, IL-17a, tumor necrosis factor (TNF)-alpha TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF); or
(vi) any combination of (i) to (v).

7. A method for producing isolated or recombinant T cells of any one of the foregoing claims, comprising:
A.
(i) inserting a DNA sequence which encodes either a PDP1 gene or a PDK1 into a vector,
(ii) contacting the vector with a T cell to form or produce a transduced T cell,
(iii) growing the transduced T cells,
(iv) harvesting and/or purifying the transduced cells,
(v) optionally expanding or permitting the harvested cells to proliferate in vitro; and
(vi) further optionally freezing said cells so as to preserve their viability; OR
B.
(i) inserting a DNA sequence which encodes either a PDP1 gene or a PDK1 into a DNA comprising a viral vector genome,
(ii) transfecting that genome into cultured mammalian cells,
(iii) culturing said cells to produce virus particles comprising said PDP1 or PDK1 gene,
(iv) harvesting and/or purifying said virus particles from the culture medium,
(v) contacting said virus particles with a T cell to form or produce a virally-transduced T cell,
(vi) growing the viral-transduced T cells,
(vii) harvesting and/or purifying the virally-transduced cells,
(viii) optionally expanding or permitting the cells to proliferate in vitro; and
(ix) and further optionally, freezing said cells so as to preserve their viability.

8. The method of claim 7A or B, wherein
(i) the vector or viral vector genome is selected from a retroviral, lentiviral, adenoviral, adeno-associated viral or herpes simplex viral vector or genome sequence; and/or
(ii) the vector or viral vector genome is a retroviral vector or retroviral genome.

9. A composition comprising isolated or recombinant T cells produced according to claim 7 and/or 8; which optionally comprises:
(i) DMSO in a concentration of 5-10% by volume, and/or
(ii) serum albumin from the same species as the recipient organism in a concentration of 0.1-5% by weight.

10. A method of treatment or prevention of a condition or disease or at least one symptom associated therewith in a subject in need thereof comprising administering a therapeutically or prophylactically effective amount isolated or recombinant T cells according to any one of claims 1-5 or a composition according to claim 6 or 9.

11. The method of claim 10, wherein
(i) the dose includes no more than about 1×10⁸ total engineered cells, e.g., in the range of about 1×10⁶ to 1×10⁸ such cells, such as 2×10⁶, 5×10⁶, 1×10⁷, 5×10⁷, or 1×10⁸ cells;
(ii) the cells are administered by intravenous, intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, intrapleural, and/or intratumoral injection or infusion;
(iii) the condition or disease is at least one cancer;
(iv) the condition or disease comprises a solid tumor, cancer reoccurrence and/or cancer metastasis;
(v) the condition or disease is at least one cancer which is selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures;
(vi) the condition or disease is at least one cancer and the treatment prevents or inhibits cancer reoccurrence;
(vii) the condition or disease is a neurodegenerative condition;
(viii) the condition or disease is a neurodegenerative condition wherein the neurodegenerative condition comprises: Adrenoleukodystrophy, Alpers' Disease, Alpha-methylacyl-CoA racemase deficiency, Alzheimer's disease and other memory disorders, Amyotrophic lateral sclerosis, Andermann syndrome, Ataxia neuropathy spectrum, Ataxia-telangiectasia, Autosomal dominant cerebellar ataxia, deafness, and narcolepsy, Balo concentric sclerosis, Batten Disease, Central pontine myelinolysis, Cerebro-Oculo-Facio-Skeletal Syndrome, Congenital insensitivity to pain with anhidrosis, Corticobasal degeneration, Creutzfeldt-Jakob disease, Familial encephalopathy with neuroserpin inclusion bodies, Fatty acid hydroxylase-associated neurodegeneration, Friedreich ataxia, Frontotemporal dementia, Gerstmann-Straussler-Scheinker disease, GM2-gangliosidosis, Hemiballismus, Hereditary sensory and autonomic neuropathy, Huntington's disease, Juvenile primary lateral sclerosis, Leigh's Disease, Lewy body disease, Marinesco-Sjogren syndrome, Meige's syndrome, Mitochondrial membrane protein-associated neurodegeneration, Monomelic Amyotrophy, Motor neuron disease, Multiple sclerosis, Multiple system atrophy, Neurodegeneration with brain iron accumulation, Neuromyelitis optica, Opsoclonus Myoclonus, Pantothenate kinase-associated neurodegeneration, Parkinson's disease, Pelizaeus-Merzbacher disease, Polycystic lipomembranous osteodysplasia with sclerosing leukoencephalopathy, Posterior cortical atrophy, Primary lateral sclerosis, Primary progressive aphasia, Progressive bulbar palsy, Progressive external ophthalmoplegia, Progressive Multifocal Leukoencephalopathy, Progressive muscular atrophy, Progressive supranuclear palsy, Pseudobulbar palsy, Riboflavin transporter deficiency neuronopathy, Sandhoff disease, Spastic paraplegia, Spinal muscular atrophy, Reye syndrome, Striatonigral degeneration, and Spasmodic torticollis;
(ix) the condition or disease is an inflammatory condition;
(x) the condition or disease is at least one inflammatory condition wherein the at least one inflammatory condition or disease is selected from single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, fibrosis, ophthalmic inflammation, dermatologic inflammation, gastrointestinal inflammation, tendinopathies, pancreatitis, hepatitis, inflammatory arthritis, multiple sclerosis, Acute Respiratory Distress Syndrome (ARDS), wound healing, diabetic ulcers, non-healing wounds, lupus, Acne vulgaris, Allergy, Asthma, Ankylosing spondylitis, Antiphospholipid antibody syndrome, Atherosclerosis, Atopic dermatitis, Autoimmune diseases, Autoimmune encephalitis, Autoinflammatory diseases, Celiac disease, Chediak-Higashi syndrome, Chronic granulomatous disease, Chronic prostatitis, Chronic recurrent multifocal osteomyelitis, Colitis, Dermatomyositis, Diverticulitis, Endometriosis, Familial Mediterranean Fever, Fatty liver disease, Glomerulonephritis, Gout, Henoch-Schonlein purpura, Hidradenitis suppurativa, Hypersensitivities, Inclusion body myositis, Inflammatory bowel diseases, Interstitial cystitis, Juvenile dermatomyositis, Juvenile idiopathic arthritis, Juvenile lupus, Juvenile vasculitis, Kawasaki disease, Lichen planus, Mast Cell Activation Syndrome, Mastocytosis, Mixed connective tissue disease, Myositis, Osteoarthritis, Otitis, Pelvic inflammatory disease, Peripheral ulcerative keratitis, Pneumonia, Polymyositis, Psoriasis, Psoriatic arthritis, Reactive arthritis, Reperfusion injury, Rheumatic fever, Rheumatoid arthritis, Rhinitis, Sarcoidosis, Scleroderma, Sjogren's syndrome, Spondyloarthritis, Systemic juvenile idiopathic arthritis, Systemic lupus erythematosus, Systemic sclerosis, Transplant rejection, Type 1 diabetes mellitus, Type 2 diabetes mellitus, Undifferentiated connective tissue disease, Uveitis, and Vasculitis, and other autoimmune diseases associated with acute or chronic inflammation;
(xi) the condition or disease is at least one autoimmune condition;
(xii) the condition or disease is at least one autoimmune condition which comprises on or more of: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease;
(xiii) the condition or disease is at least one infectious condition;
(xiv) the condition or disease is at least one infectious condition which comprises one or more of an infection comprising Helicobacter pylori, Borrelia burgdorferi, Legionella pneumophilia, Mycobacteria sp (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, Actinomyces israeli, and/or Chlamydia trachomatis;
(xv) the condition or disease is at least one infectious condition wherein the infectious condition comprises an infection comprising Retroviridae (for example, HIV), Picornaviridae (for example, polio viruses, hepatitis A virus; enteroviruses, human coxsackie viruses, rhinoviruses, echoviruses), Caliciviridae (such as strains that cause gastroenteritis), Togaviridae (for example, equine encephalitis viruses, rubella viruses); Flaviridae (for example, dengue viruses, encephalitis viruses, yellow fever viruses), Coronaviridae (for example, coronaviruses); Rhabdoviridae (for example, vesicular stomatitis viruses, rabies viruses); Filoviridae (for example, Ebola viruses), Paramyxoviridae (for example, parainfluenza viruses, mumps virus, measles virus, respiratory syncytial virus), Orthomyxoviridae (for example, influenza viruses), Bunyaviridae (for example, Hantaan viruses, bunya viruses, phleboviruses and Nairo viruses), Arenaviridae (hemorrhagic fever viruses), Reoviridae (e.g., reoviruses, orbiviurses and rotaviruses); Birnaviridae; Hepadnaviridae (Hepatitis B virus) Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses, Epstein-Barr virus, Poxviridae (variola viruses, vaccinia viruses, pox viruses), and Asfarviridae (such as African swine fever virus), and/or Norovirus;
(xvi) the condition or disease is at least one infectious condition wherein the infectious condition comprises an infection comprising Aspergillus sp., Candida albicans, Cryptococcus, Histoplasma, Cryptococcus neoformans, Histoplasma capsulatum, Coccidioides immitis, and/or Blastomyces dermatitidis;
(xvii) the condition or disease is at least one infectious condition wherein the infectious condition comprises an infection comprising Plasmodium falciparum: or Toxoplasma gondii;
(xviii) the condition or disease comprises an inflammatory condition or disease which comprises an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS; or
(xix) any combination of (i) to (xviii).
